(19) United States
(12) Patent Application Publication
(10) Pub. No.: US 2024/0269270 A1
(43) Pub. Date: Aug. 15, 2024

(54) COVID-19 VACCINES WITH TOCOPHEROL-CONTAINING SQUALENE EMULSION ADJUVANTS

(71) Applicants: SANOFI PASTEUR INC., Swiftwater, PA (US); GLAXOSMITHKLINE BIOLOGICALS SA, Rixensart (BE)

(72) Inventors: Natalie ANOSOVA, Lexington, MA (US); Salvador Fernando AUSAR, Markham (CA); Catherine BERRY, Lyon (FR); Florence BOUDET, Lyon (FR); Thomas BREUER, Wavre (BE); Danilo CASIMIRO, Harleysville, PA (US); Roman M. CHICZ, Belmont, MA (US); Gustavo DAYAN, Bethlehem, PA (US); Guy DE BRUYN, Bushkill Township, PA (US); Carlos DIAZGRANADOS, Wind Gap, PA (US); Tong-Ming FU, Philadelphia, PA (US); Marie GARINOT, Lyon (FR); Lorry GRADY, Simsbury, CT (US); Sanjay GURUNATHAN, Bethlehem, PA (US); Kirill KALNIN, Pelham, NH (US); Nikolai KHRAMTSOV, Branford, CT (US); Valérie LECOUTURIER, Chazay d'Azergues (FR); Nausheen RAHMAN, North York (CA); Sophie RUIZ, Lyon (FR); Stephen SAVARINO, Saylorsburg, PA (US); Saranya SRIDHAR, London (GB); Indresh K. SRIVASTAVA, Marlborough, CT (US); James TARTAGLIA, Nazareth, PA (US); Timothy TIBBITTS, East Greenwich, RI (US)

(73) Assignees: SANOFI PASTEUR INC., Swiftwater, PA (US); GLAXOSMITHKLINE BIOLOGICALS SA, Rixensart (BE)

(21) Appl. No.: 18/042,637

(22) PCT Filed: Aug. 23, 2021
(86) PCT No.: PCT/US2021/047149

§ 371 Date: Feb. 23, 2023

Related U.S. Application Data
(60) Provisional application No. 63/069,171, filed on Aug. 24, 2020.
(60) Provisional application No. 63/215,092, filed on Jun. 25, 2021.
(60) Provisional application No. 63/189,044, filed on May 14, 2021.
(60) Provisional application No. 63/184,155, filed on May 4, 2021.

(51) Int. Cl. A61K 39/39; A61K 39/00; A61K 39/215; A61P 37/04; C07K 14/005; C12N 5/07; C12N 7/00

(52) U.S. Cl. CPC A61K 39/39; A61K 39/215; A61P 37/04; C07K 14/005; C12N 5/0601; C12N 7/00; A61K 2039/545; A61K 2039/55566; A61K 2039/70; C12N 2770/20022; C12N 2770/20034; C12N 2770/20071

(57) ABSTRACT

Provided are novel vaccines for prophylactic treatment of SARS-COV-2 infections and COVID-19 and methods of making the vaccines, wherein the vaccines contain an oil-in-water emulsion comprising tocopherol and squalene.

### CROSS-REFERENCE TO RELATED APPLICATIONS

This application claims priority from U.S. Provisional Applications 63/069,171, filed Aug. 24, 2020; 63/184,155, filed May 4, 2021: 63/189,044, filed May 14, 2021; and 63/215,092, filed Jun. 25, 2021. The disclosures of the aforementioned priority applications are incorporated herein by reference in their entirety.

### FEDERALLY SPONSORED RESEARCH OR DEVELOPMENT

This invention was made with government support under HHSO100201600005I awarded by U.S. Department of Health and Human Services and ASPR-BARDA; and an Other Transaction Agreement (OTA) W15QKN-16-9-1002 issued by the U.S. Army Contracting Command, ACC-NJ and awarded as a joint mission between the Department of Health and Human Services and the Department of Defense. The government has certain rights in the invention.

### SEQUENCE LISTING

The instant application contains a Sequence Listing which has been submitted electronically in ASCII format and is hereby incorporated by reference in its entirety. The electronic copy of the Sequence Listing, created on Aug. 10, 2021, is named 025532_WO005_SL.txt and is 48,845 bytes in size.

### BACKGROUND OF THE INVENTION

Coronaviruses are a family of enveloped, positive-sense, single-stranded RNA viruses that infect a wide variety of mammalian and avian species. The viral genome is packed into a capsid that is comprised of the viral nucleocapsid (N) protein and surrounded by a lipid envelope. Embedded in the lipid envelope are the membrane (M) protein, the envelope small membrane (E) protein, hemagglutinin-esterase (HE), and the spike (S) protein. The S protein mediates viral attachment and entry into cells.

Human coronaviruses (hCoVs) cause respiratory illnesses. Low pathogenic hCoVs infect the upper respiratory tract and cause mild colds. Highly pathogenic hCoVs predominantly infect lower airways and can cause severe, and sometimes fatal, pneumonia such as severe acute respiratory syndrome (SARS-COV) and Middle East respiratory syndrome (MERS-COV). Severe pneumonia caused by hCoVs is typically associated with rapid virus replication, massive inflammatory cell infiltration, and elevated pro-inflammatory cytokines and chemokines, resulting in acute lung injury and acute respiratory distress syndrome (see. e.g., Channappanavar and Perlman, Semin Immunopathol (2017) 39(5):529-39).

Severe acute respiratory syndrome coronavirus 2 (SARS-COV-2), also known as the 2019 novel coronavirus (2019-nCOV), is the seventh known coronavirus to infect humans, after HCoV-229E, HCoV-NL63, HCoV-OC43, HCoV-HKU1, MERS-COV, and the original SARS-COV (Zhu et al., N Eng Med. (2020) 382 (8):727-33). Like the SARS-related coronavirus strain implicated in the 2003 SARS outbreak, SARS-COV-2 is a member of the subgenus Sarbecovirus (Beta-CoV lineage B). SARS-COV-2 is the cause of the ongoing 2019-21 coronavirus disease (COVID-19) (Chan et al., Lancet (2020) 395(10223):514-23; Xu et al., Lancet Respir Med. (2020) doi: 10.1016/S2213-2600(20)30076-X); GenBank: MN908947.3: Gorbalenya et al., bioRxiv (2020) doi: 10.1101/2020.02.07.937862). Human-to-human transmission occurs primarily via respiratory droplets and aerosols.

The clinical profile of COVID-19 varies. In the majority of cases, infected individuals may be asymptomatic or have mild symptoms. Among those with symptoms, typical presentations include fever, cough, shortness of breath, anosmia, and fatigue. More severe manifestations include acute respiratory distress syndrome, strokes, and cytokine release syndrome, in some cases resulting in death. Severe illness can occur in healthy individuals of any age, but it predominantly occurs in adults with advanced age or underlying medical comorbidities. Older adults are most commonly affected and suffer a high mortality rate. Comorbidities and other conditions associated with severe illness and mortality include chronic kidney disease, chronic obstructive pulmonary disease (COPD), an immunocompromised state, obesity, serious heart conditions (e.g., heart failure, coronary artery disease, or cardiomyopathies), sickle cell disease, diabetes, hypertension, liver disease, and pulmonary fibrosis. The risk from COVID-19 also varies by country and regionally within countries around the world (see. e.g., de Souza, Nat Hum Behav. (2020) 4:856-865; Chen, Cell Death Dis. (2020) 11:438).

SARS-COV-2 infects cells through binding to the cell surface protein angiotensin-converting enzyme 2 (ACE2) (Hoffmann et al., Cell (2020) 181(2):271-80; Walls et al., Cell (2020) 181(2):281-92). The virus gains entry into host cells through the S protein. The S protein is a class I fusion protein and is heavily coated with polysaccharides that help the virus evade immune surveillance. The protein is produced through processing of precursor S polypeptides. The precursor polypeptide undergoes glycosylation, removal of the signal peptide, and cleavage by proprotein convertase furin between residues 685 and 686 to produce to two subunits, S1 and S2. S1 and S2 remain associated as a protomer. The S protein is a trimer of the protomer, existing in a metastable prefusion conformation. Upon binding of the S1 subunit to the host cell receptor, the S1 subunit is released from the protein. The remaining S2 subunit transits into a highly stable postfusion conformation and facilitates membrane fusion between the virus and the host cell and hence viral entry into the cell (see. e.g., Wrapp et al., Science (2020) 10.1126/science.abb2507; Shang et al., PNAS (2020) 117(21): 11727-34).

The S protein is a key target for vaccine development. It is expected that the protein in the prefusion conformation presents the most neutralization-sensitive epitopes (see. e.g., Wrapp, supra). Successful immunization strategies require stable antigens, and attempts to stabilize the SARS-COV-2 S protein in the prefusion conformation have been described (see. e.g., Xiong et al., Nat Struct Mol Biol. (2020) doi.org/10.1038/s41594-020-0478-5).

The public health crisis caused by COVID-19 continues unabated, especially in developing countries. Variants of SARS-COV-2 continue to emerge. There remains an urgent need to develop efficacious vaccines that can help combat the continued threat of COVID-19.

### SUMMARY OF THE INVENTION

The present disclosure provides an immunogenic composition comprising (a) one, two, three, or more recombinant SARS-COV-2 proteins, wherein one or more of the proteins is a trimer of a polypeptide comprising, from N terminus to C terminus, (i) a sequence that is at least 94%, for example, at least 95% (e.g., at least 96, 97, 98, or 99%) identical to (1) residues 19-1243 of SEQ ID NO:10, or (2) residues 19-1240 of SEQ ID NO:13, wherein residues GSAS (SEQ ID NO:6) at positions 687-690 of SEQ ID NO:10 (and corresponding positions in SEQ ID NO: 13) and residues PP at positions 991 and 992 of SEQ ID NO:10 (and corresponding positions in SEQ ID NO:13) are maintained in the sequence; and (ii) a trimerization domain, wherein the trimerization domain comprises SEQ ID NO:7; and (b) an adjuvant, wherein the adjuvant is an oil-in-water emulsion comprising tocopherol and squalene. When a composition is said to have two or more proteins, it is intended that these proteins are different from each other.

In another aspect, the present disclosure provides an immunogenic composition comprising (a) one, two, three or more recombinant SARS-COV-2 S proteins, each obtainable by a method comprising introducing into insect cells a baculoviral vector for expressing a polypeptide comprising, from N terminus to C terminus, (i) a signal peptide derived from an insect or baculoviral protein (e.g., a chitinase), (ii) a sequence that is at least 95% (e.g., at least 96, 97, 98, or 99%) identical to (1) residues 19-1243 of SEQ ID NO:10 or (2) residues 19-1240 of SEQ ID NO:13, wherein residues GSAS (SEQ ID NO:6) at positions 687-690 of SEQ ID NO:10 (and corresponding positions in SEQ ID NO: 13) and residues PP at positions 991 and 992 of SEQ ID NO: 10 (and corresponding positions in SEQ ID NO: 13) are maintained in the sequence; and (iii) a trimerization domain, wherein the trimerization domain comprises SEQ ID NO:7, culturing the insect cells under conditions that allow expression and trimerization of the polypeptide, and isolating the recombinant SARS-COV-2 S protein from the culture, wherein the recombinant SARS-COV-2 S protein is a trimer of the polypeptide, without the signal sequence; and (b) an adjuvant, wherein the adjuvant is an oil-in-water emulsion comprising tocopherol and squalene. In some embodiments, the baculoviral expression vector comprises a polyhedrin promoter linked operably to a coding sequence for the polypeptide. In some embodiment, the signal peptide is derived from an insect or baculoviral chitinase. In some embodiments, the signal peptide comprises SEQ ID NO:3.

In some embodiments, the immunogenic composition comprises one (monovalent) or more (multivalent) different recombinant SARS-COV-2 S proteins. For example, the composition comprises two (bivalent), three (trivalent), or four (quadrivalent) different recombinant SARS-COV-2 S proteins.

In some embodiments, the recombinant polypeptide comprises or has a sequence identical to (i) residues 19-1243 of SEQ ID NO: 10 or (ii) residues 19-1240 of SEQ ID NO:13. In some embodiments, the composition is bivalent and comprises a recombinant protein that is a trimer of the recombinant polypeptide comprising or having a sequence identical to residues 19-1243 of SEQ ID NO: 10 and a trimer of the recombinant polypeptide comprising or having a sequence identical to residues 19-1240 of SEQ ID NO:13, optionally in equal amounts.

In some embodiments, each dose (in, e.g., about 0.2, 0.25, 0.3, 0.4, 0.5, 0.6, or 0.7 mL) of the immunogenic composition comprises or is prepared by mixing: (i) an antigen component comprising about 2 μg to about 50 μg, optionally about 2.5 μg to about 50 μg or about 5 μg to about 50 μg, of each of the recombinant SARS-COV-2 S protein(s); and (ii) an oil-in-water emulsion adjuvant comprising a) 10.69 mg squalene, 4.86 mg polysorbate 80, and 11.86 mg α-tocopherol in a phosphate-buffered saline, optionally as shown in Table 9, further optionally wherein the adjuvant is provided in 0.25 mL, b) 5.34 mg squalene. 2.43 mg polysorbate 80 and 5.93 mg α-tocopherol in a phosphate-buffered saline, optionally as shown in Table 9, further optionally wherein the adjuvant is provided in 0.25 mL, c) 2.67 mg squalene, 1.22 mg polysorbate 80 and 2.97 mg α-tocopherol in a phosphate-buffered saline, optionally as shown in Table 9, further optionally wherein the adjuvant is provided in 0.25 mL, or d) 1.34 mg squalene, 0.61 mg polysorbate 80 and 1.48 mg α-tocopherol in a phosphate-buffered saline, optionally as shown in Table 9, further optionally wherein the adjuvant is provided 0.25 mL. Alternatively, the aforementioned protein amounts are the total protein amount in the composition.

In some embodiments, each dose (in, e.g., about 0.5 mL) of the immunogenic composition is prepared by mixing 0.25 mL of an antigen component and 0.25 mL of the AS03 adjuvant (AS03_{A}). The 0.25 mL of the antigen component may comprise or be prepared by mixing 0.0975 mg monobasic sodium phosphate monohydrate, 0.65 mg dibasic sodium phosphate dodecahydrate (corresponding to 0.26 mg dibasic sodium phosphate anhydrous), 2.2 mg sodium chloride, 50-600 (e.g., 55 or 550) μg polysorbate 20, and about 0.25 mL water (q.s. ad 0.25 mL). In certain embodiments, the 0.25 mL of the antigen component, or the 0.5 mL of the final immunogenic composition (with adjuvant) comprises 2.5 μg of each of the recombinant SARS-COV-2 S protein(s), optionally wherein the composition comprises two different recombinant SARS-COV-2 S proteins in equal amounts; if the composition is monovalent, it comprises 2.5 μg of the recombinant S protein. In certain embodiments, the 0.25 mL of the antigen component, or the 0.5 mL of the final immunogenic composition (with adjuvant) comprises 5 μg of each of the recombinant SARS-COV-2 S protein(s), optionally wherein the composition comprises two different recombinant SARS-CoV-2 S proteins in equal amounts: if the composition is monovalent, it comprises 5 μg of the recombinant S protein. In certain embodiments, the 0.25 mL of the antigen component, or the 0.5 mL of the final immunogenic composition (with adjuvant) comprises 10 μg of each of the recombinant SARS-COV-2 S protein(s), optionally wherein the composition comprises two different recombinant SARS-COV-2 S proteins in equal amounts: if the composition is monovalent, it comprises 10 μg of the recombinant S protein. Alternatively, the aforementioned protein amounts are the total protein amount in the composition, rather than the protein amount of each protein in the composition.

In another aspect, the present disclosure provides a container containing the immunogenic composition described herein in a single dose or multiple doses, with each dose in a volume of e.g., about 0.25 or about 0.5 mL. In some embodiments, the container is a vial or a syringe.

In another aspect, the present disclosure provides a kit for intramuscular vaccination, wherein the kit comprises two containers, wherein (a) a first container contains a pharmaceutical composition comprising one, two, three, or more recombinant SARS-COV-2 S proteins, wherein one or more of the proteins is a trimer of a polypeptide comprising, from N terminus to C terminus, (i) a sequence that is at least 94%, for example at least 95% (e.g., at least 96, 97, 98, or 99%) identical to (A) residues 19-1243 of SEQ ID NO:10, wherein residues GSAS (SEQ ID NO:6) at positions 687-690 of SEQ ID NO: 10 and residues PP at positions 991 and 992 of SEQ ID NO: 10 are maintained in the sequence or (B) residues 19-1240 of SEQ ID NO:13, wherein residues GSAS (SEQ ID NO:6) at positions 684-687 of SEQ ID NO: 13 and residues PP at positions 988 and 989 of SEQ ID NO:13 are maintained in the sequence; and (ii) a trimerization domain, wherein the trimerization domain comprises SEQ ID NO:7; and (b) a second container contains an oil-in-water adjuvant comprising tocopherol and squalene. In some embodiments, the polypeptide comprises or has a sequence identical to (i) residues 19-1243 of SEQ ID NO:10, or (ii) residues 19-1240 of SEQ ID NO:13. In further embodiments, the container contains two different recombinant S proteins, each having one of the aforementioned sequences. In some embodiments, the first container comprises one or more doses of the recombinant SARS-COV-2 S protein(s) provided in a phosphate-buffered saline, optionally as shown in Table A, Table 8 or Table 12, wherein each dose (0.25 mL in volume) has about 2.5 μg to 45 μg, optionally 5 μg to 45 μg (e.g., 2.5, 5, 10, 15, or 45 μg) of the recombinant S protein(s) (in total or separately). In some embodiments, the second container comprises one or more doses of the adjuvant, wherein each dose of the adjuvant is 0.25 mL in volume comprising a) 10.69 mg squalene, 4.86 mg polysorbate 80, and 11.86 mg α-tocopherol in a phosphate-buffered saline, optionally as shown in Table 9, b) 5.35 mg squalene, 2.43 mg polysorbate 80, and 5.93 mg α-tocopherol in a phosphate-buffered saline, optionally as shown in Table 9, c) 2.67 mg squalene, 1.22 mg polysorbate 80, and 2.97 mg α-tocopherol in a phosphate-buffered saline, optionally as shown in Table 9, or d) 1.34 mg squalene, 0.61 mg polysorbate 80, and 1.48 mg α-tocopherol in a phosphate-buffered saline, optionally as shown in Table 9.

The present disclosure also provides a method of making a vaccine kit, comprising providing the recombinant S protein(s) and the adjuvant of the immunogenic composition and packaging the protein and the adjuvant into separate sterile containers.

The present disclosure further provides a method of preventing or ameliorating COVID-19 in a subject in need thereof, comprising administering to the subject a prophylactically effective amount of the immunogenic composition herein. In some embodiments, the prophylactically effective amount is administered in a single dose or in two or more doses. In some embodiments, the prophylactically effective amount is about 5 to about 50 μg per dose, optionally 5, 10, 15, or 45 μg per dose, of the recombinant SARS-COV-2 S protein(s), administered intramuscularly in a single dose or in two or more doses. In some embodiments, two or more doses of the immunogenic composition are administered with an interval of about two weeks to about three months (e.g., about three weeks or about 21 days), wherein each dose of the immunogenic composition comprises 5 μg or 10 μg of the recombinant SARS-COV-2 S protein(s). In some embodiments, the subject is administered the immunogenic composition herein in accordance with regimen 1, 10, 11, or 12 in Table B, infra.

In some embodiments, the present immunogenic composition is used as a booster vaccine, e.g., to prevent or ameliorating COVID-19, in a subject with a previous SARS-COV-2 infection, or in a subject who has been vaccinated with a COVID-19 vaccine against the same or different viral strain. The first COVID-19 vaccine may be a killed vaccine, a subunit vaccine, or a genetic vaccine (e.g., an mRNA or viral vector vaccine). In certain embodiments, the subject has been vaccinated with a genetic vaccine comprising an mRNA that encodes a recombinant SARS-COV-2 S antigen. In certain embodiments, the present immunogenic composition is administered to the subject about 4 weeks, about one month, about three months, about six months, about four to ten months, or about one year post-infection or after the subject is vaccinated with the first COVID-19 vaccine, optionally wherein the immunogenic composition comprises 2.5 μg or 5 μg of the recombinant SARS-COV-2 protein(s), and further optionally wherein the immunogenic composition is monovalent or multivalent. In further embodiments, the booster shot is given about eight months after the completion of the first COVID-19 vaccination. In some embodiments, the genetic vaccine comprises an mRNA that encodes a recombinant SARS-COV-2 S antigen, optionally wherein the recombinant SARS-COV-2 S protein comprises SEQ ID NO:1, 4, 10, 13, or 14, or an antigenic fragment thereof. In certain embodiments, the booster immunogenic composition herein comprises 2.5 or 5 μg, per dose, of the recombinant SARS-CoV-2 S protein(s). In certain embodiments, the booster immunogenic composition is monovalent (e.g., comprising a recombinant S protein comprising SEQ ID NO: 10 or 13, without the signal sequence) or bivalent (e.g., comprising a first recombinant S protein comprising SEQ ID NO: 10, without the signal sequence, and a second recombinant S protein comprising SEQ ID NO: 13, without the signal sequence). In some embodiments, the subject is administered the immunogenic composition herein in accordance with regimen 2, 3, 4, 5, 6, 7, 8, or 9 in Table B, infra.

The present disclosure also provides the immunogenic composition described herein for use in prophylactic treatment of COVID-19.

The present disclosure further provides the use of the immunogenic composition described herein for the manufacture of a medicament for prophylactic treatment of COVID-19. The prophylactic treatment may prevent or ameliorate COVID-19 in a subject in need thereof that is described herein.

In certain embodiments, in the immunogenic composition described herein, the recombinant S proteins are used in conjunction with the tocopherol-containing squalene emulsion adjuvant herein in prophylactic treatment of COVID-19: the tocopherol-containing squalene emulsion adjuvant described herein is used in conjunction with the recombinant S proteins in prophylactic treatment of COVID-19: use of the recombinant S proteins and the adjuvant for the manufacture of an article of manufacture (such as a vaccination kit, e.g., a vaccination kit for intramuscular injection) for use in prophylactic treatment of COVID-19.

The following embodiments apply to (i) the immunogenic composition described herein, (ii) any use thereof, (iii) the container kit described herein, (iv) the prophylactic and therapeutic uses of the immunogenic composition and of the container kit, and (v) the methods of preventing or ameliorating COVID-19 in a subject in need thereof described herein.

In certain embodiments, the tocopherol is alpha-tocopherol, optionally D/L-alpha-tocopherol.

In certain embodiments, the adjuvant has an average droplet size of less than 1 μm, optionally wherein the average droplet size is less than 500 nm, less than 200 nm, 50 to 200 nm, 120 to 180 nm, or 140 to 180 nm.

In certain embodiments, the adjuvant has a poly dispersity index of 0.5 or less, such as 0.3 or less, or 0.2 or less.

In certain embodiments, the adjuvant comprises a surfactant selected from poloxamer 401, poloxamer 188, polysorbate 80, sorbitan trioleate, sorbitan monooleate and polyoxyethylene 12 cetyl/stearyl ether, either alone, or in combination with each other or in combination with other surfactants.

In certain embodiments, the adjuvant comprises a surfactant selected from polysorbate 80, sorbitan trioleate, sorbitan monooleate, and polyoxyethylene 12 cetyl/stearyl ether either alone, or in combination with each other.

In certain embodiments, the adjuvant comprises polysorbate 80.

In certain embodiments, the adjuvant comprises one, two, or three surfactants.

In certain embodiments, the weight ratio of squalene to tocopherol in the adjuvant is 0.1 to 10, optionally 0.2 to 5, 0.3 to 3, 0.4 to 2, 0.72 to 1.136, 0.8 to 1, 0.85 to 0.95, or 0.9.

In certain embodiments, the weight ratio of squalene to surfactant in the adjuvant is 0.73 to 6.6, optionally 1 to 5, 1.2 to 4, 1.71 to 2.8, 2 to 2.4, 2.1 to 2.3, or 2.2.

In certain embodiments, the human subject is a child, an adult, or an elderly adult.

In certain embodiments, the amount of squalene in a single dose of the adjuvant is at least 1.2 mg, optionally 1.2 to 20 mg, 1.2 to 15 mg. 1.2 to 2 mg, 1.21 to 1.52 mg, 2 to 4 mg, 2.43 to 3.03 mg, 4 to 8 mg, 4.87 to 6.05 mg, 8 to 12.1 mg, or 9.75 to 12.1 mg.

In certain embodiments, the amount of tocopherol in a single dose of the adjuvant is at least 1.3 mg, optionally 1.3 to 22 mg, 1.3 to 16.6 mg, 1.3 to 2 mg, 1.33 to 1.69 mg, 2 to 4 mg. 2.66 to 3.39 mg, 4 to 8 mg, 5.32 to 6.77 mg, 8 to 13.6 mg, or 10.65 to 13.53 mg.

In certain embodiments, the amount of surfactant in a single dose of the adjuvant is at least 0.4 mg, optionally 0.4 to 9.5 mg. 0.4 to 7 mg, 0.4 to 1 mg, 0.54 to 0.71 mg, 1 to 2 mg, 1.08 to 1.42 mg, 2 to 4 mg, 2.16 to 2.84 mg, 4 to 7 mg, or 4.32 to 5.68 mg.

In certain embodiments, the adjuvant comprises or consists essentially of squalene: tocopherol, optionally D/L-alpha-tocopherol: surfactant, optionally polysorbate 80; and water.

In certain embodiments, the volume of a single dose of the immunogenic composition for intramuscular injection is 0.05 mL to 1 mL, optionally 0.1 to 0.6 mL, 0.2 to 0.3 mL, 0.25 mL, 0.4 to 0.6 mL, or 0.5 mL.

In certain embodiments, the immunogenic composition, or a mixture of the contents of the first and second containers, has a pH of 4 to 9, optionally 5 to 8.5, 5.5 to 8, or 6.5 to 7.4.

In certain embodiments, the immunogenic composition, or a mixture of the contents of the first and second containers, has an osmolality of 250 to 750 mOsm/kg, optionally 250 to 550 mOsm/kg, 270 to 500 mOsm/kg, or 270 to 400 mOsm/kg.

In certain embodiments, the immunogenic composition, or a mixture of the contents of the first and second containers, comprises squalene at 0.8 to 100 mg per mL, optionally 1.2 to 48.4 mg per mL, 10 to 30 mg per mL, or 21.38 mg per mL.

In certain embodiments, the volume of a single dose of the adjuvant for intramuscular injection, prior to being mixed with the recombinant S protein, is 0.05 mL to 1 mL, optionally 0.1 to 0.6 mL, 0.2 to 0.3 mL, 0.25 mL, 0.4 to 0.6 mL, or 0.5 mL.

In certain embodiments, the adjuvant, prior to being mixed with the recombinant S protein has a pH of 4 to 9, optionally 5 to 8.5, 5.5 to 8, or 6.5 to 7.4: has an osmolality of 250 to 750 mOsm/kg, optionally 250 to 550 mOsm/kg, 270 to 500 mOsm/kg, or 270 to 400 mOsm/kg: comprises a buffer and/or tonicity modifying agents, optionally a modified phosphate-buffered saline: has a squalene concentration of 0.8 to 100 mg per mL, optionally 1.2 to 48.4 mg per ml; and has a single dose volume of 0.05 mL to 1 mL, optionally 0.1 to 0.6 mL.

In certain embodiments, the recombinant S protein is provided in an aqueous liquid solution that has, prior to being mixed with the adjuvant, a single dose volume of 0.2 to 0.3 mL, optionally 0.25 mL, 0.4 to 0.6 mL, or 0.5 ml: a pH of 4 to 9, optionally 5 to 8.5, 5.5 to 8, or 6.5 to 7.4; and an osmolality of 250 to 750 mOsm/kg, optionally 250 to 550 mOsm/kg. 270 to 500 mOsm/kg, or 270 to 400 mOsm/kg.

The disclosed embodiments are suitable for treating a subject which is not infected with SARS-COV-2. The disclosed embodiments are suitable for eliciting in a human subject in need thereof an immune response that reduces partially or completely the severity of one or more symptoms and/or time over which one or more symptoms are experienced by the subject, reduces the likelihood of developing an established infection after challenge, slows progression of illness, optionally extending survival, produces neutralizing antibodies to SARS-COV-2, and/or is a SARS-COV-2 S protein specific T cell response.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a diagram showing the design for Construct 1, which contains a baculoviral expression cassette for a recombinant SARS-COV-2 S protein. The expression cassette includes a polyhedrin promoter and a coding sequence for a polypeptide containing a chitinase signal sequence (“ss”) and a SARS-COV-2 S protein ectodomain containing mutations at a putative furin cleavage site at the S1/S2 junction and a double proline substitution in the S2 subunit.

FIG. 2A is a schematic to depict the assembly of the SapI digested pPSC12DB-LIC transfer plasmid with synthesized gBlock Fragments. The SapI linearized transfer plasmid is shown in grey, polyhedrin promoter green arrow, gBlock fragments colored yellow, blue and orange, and each overlapping sequence is depicted as identical colors (top panel). The final transfer plasmid containing the preS dTM gene is shown in the bottom panel.

FIG. 2B shows the 5′ and 3′ end sequences of the gBlock Fragments (SEQ ID NOs:14-23, respectively, in order of appearance).

FIG. 3 is a diagram illustrating the process for generating a baculoviral construct for expressing a recombinant SARS-COV-2 S protein. MV: Master Virus.

FIG. 4 is a plot showing the D21 and D36 serum S-specific IgG levels in mice injected on DO/D21 with preS dTM and S dTM without adjuvant. Titers are expressed as inverse of dilution for OD=0.2. EU: ELISA units. preS dTM: a recombinant stabilized, prefusion SARS-COV-2 S protein with deleted transmembrane and cytoplasmic domains (SEQ ID NO:10). S dTM: a recombinant, non-stabilized SARS-COV-2 S protein with deleted transmembrane and cytoplasmic domains.

FIG. 5 is a plot showing the effect of adjuvant AS03 on S-specific IgG levels in injected mice on Day 21 and Day 36. Titers are expressed as inverse of dilution for OD=0.2. Light grey squares and triangles: D21. Dark grey squares and triangles: D36.

FIG. 6A is a plot showing PRNT50 titers of serum antibodies obtained from immunized mice on D36. The lower horizontal dashed line indicates lower limit of quantitation (LLOQ), which is ½ the starting dilution.

FIG. 6B shows the 50% inhibitory concentration (IC₅₀) titers of neutralization antibodies against the Integral Molecular SARS-COV-2 S pseudovirus displaying SARS-CoV-2 S protein from the same study as FIG. 6A.

FIG. 7 is a plot of percentages of CD4⁺ T cells expressing IFN-γ, TNF-α, IL-2, IL-4 and IL-5 cytokines in response to stimulation with S1 peptide pool in the AS03-adjuvanted preS dTM and naïve control groups (bars=mean %).

FIG. 8 is a plot showing levels of serum IgG against SARS-COV-2 prefusion S protein in Rhesus macaques that were immunized with a targeted 15 μg of preS dTM with or without the AS03 adjuvant. The IgG levels were measured on DO, D21, and D28. “-” on the X-axis indicates vehicle control. Y axis represents log scale of EU.

FIG. 9 is a plot showing the 50% inhibitory concentration (IC₅₀) titers of neutralization antibodies against the Integral Molecular SARS-COV-2 S pseudovirus displaying SARS-COV-2 S protein from the same study as FIG. 8. The Y-axis represents the Log₁₀ values of the IC₅₀ titers. “Conv”: human SARS-COV-2 convalescent serum (high titer).

FIG. 10 is a plot showing the 50% micro-neutralization (MN₅₀) titers of neutralization antibodies against wildtype SARS-COV-2 virus from the same study as FIG. 8. The Y-axis represents the Log₁₀ values of the MN₅₀ titers in microneutralization (MN) assays.

FIG. 11 is a plot showing levels of serum IgG against SARS-COV-2 prefusion S protein in hamsters that were immunized either once or twice with a targeted 2.25 μg dose of preS dTM with or without adjuvant versus placebo. The IgG levels were measured on D35 (14 days post dose 1 for the one-dose cohort and 14 days post dose 2 for the two-dose cohort). Titers are expressed as inverse of dilution for OD=0.2. The lower horizontal dashed line the inverse of the lowest dilution tested. Y axis represents Log₁₀ scale of EU.

FIG. 12 is a graph showing the ID₅₀ titers of neutralization antibodies against the Integral Molecular SARS-COV-2 S pseudovirus displaying SARS-COV-2 S protein in hamsters that were immunized either once or twice with a targeted 2.25 μg dose of preS dTM with or without adjuvant versus placebo. The pseudovirus neutralization antibody levels were measured on D35 (14 days post dose 1 for the one-dose cohort and 14 days post dose 2 for the two-dose cohort). The lower horizontal dashed line indicates the inverse of the lowest dilution tested. One hamster from the placebo group was removed from analysis due to a technical issue.

FIG. 13 is a pair of plots showing the percent body weight changes in hamsters that were immunized either once (one-dose cohort, left plot) or twice (two-dose cohort, right plot) with a targeted 2.25 μg dose of preS dTM with or without the AS03 adjuvant versus placebo for Days 0-4 after challenge with SARS-COV-2 USA/WA1/2020 (P2) strain.

FIG. 14 is a pair of plots showing the total viral load in the lungs (left plot) and nares (right plot) in the two-dose cohort of hamsters immunized with a targeted 2.25 μg dose of preS dTM with or without adjuvant versus placebo then challenged 35 days post-last immunization with 2.3E4 PFU of SARS-COV-2 USA/WA1/2020 strain. The Y axis shows genome copies/gram on a Log₁₀ scale on D4 and D7 post challenge.

FIG. 15 is a pair of plots showing the subgenomic viral load in the lungs (left plot) and nares (right plot) in the two-dose cohort of hamsters immunized with a targeted 2.25 μg dose of preS dTM with or without adjuvant versus placebo then challenged 35 days post-last immunization with 2.3E4 PFU of SARS-COV-2 USA/WA1/2020 strain. The Y axis shows genome copies/gram on a Log₁₀ scale on D4 and D7 post challenge.

FIG. 16A is a pair of plots showing lung pathology scoring in hamsters that were immunized with a targeted 2.25 μg dose of preS dTM with or without adjuvant versus placebo then challenged 35 days post-last immunization with SARS-COV-2 USA/WA1/2020 strain. Each dot represents a single hamster. The Y axis shows lung pathology scores on a scale of 0) (normal) to 3 (severe). The bar represents the group median.

FIG. 16B is a plot showing individual daily body weight losses (%) in naïve and recombinant S immunized hamsters after challenge with the Beta variant. Symbols represent individual data and lines the mean of the group.

FIG. 17 is a bar graph showing the incidence rates of solicited reactions after any dose of preS dTM in all age groups.

FIG. 18 is a pair of bar graphs showing the incidence rates of solicited reactions after any dose of preS dTM by age group.

FIG. 19 is a bar graph showing the reactogenicity of three doses of preS dTM (5, 10, and 15 μg) post-injection 2 (“PD2”). VAT02: Phase II clinical trial.

FIG. 20 is a pair of graphs showing individual D614 pseudovirion (PsV) neutralizing antibody (Nab) titers (Log₁₀) measured using VSV-PsV (Nexelis, left panel) or lentivirus-PsV (SP REI, right panel) neutralizing assays at week 5 in cynomolgus macaques from the CoV2-06_NHP study. Thick bars represent the mean of the group, the dotted line is the inverse of the lowest dilution tested, and the LLOQ of the VSV-PsV assay is 1.5 Log₁₀. The fold-change for all 3 dose levels combined are indicated below the graphs.

FIG. 21 is a graph showing the individual lentivirus-PsV NAb titers (Log₁₀) against variants of concern at week 5 in cynomolgus macaques. Dotted line represents the inverse of the lowest dilution tested. CoV2 preS dTM-AS03: recombinant S protein(s) derived from the Wuhan strain (D614) and/or the B.1.351 (South African) variant strain formulated with AS03 as described herein.

FIG. 22 is a pair of graphs showing individual S-binding IgG titers (Log₁₀ EU) before and after the booster immunization, in mRNA-primed macaques (left panel), subunit-primed macaques (middle panel) and human convalescent sera (right panel). Lines and bars represent the mean of the group and horizontal dotted lines the inverse of the lowest dilution tested.

FIG. 23 is a panel of graphs showing individual D614G (top) and B. 1.351 (bottom) PsV NAb titers (Log₁₀) before and after a booster immunization in mRNA-primed (left panel) and subunit-primed (middle panel) macaques, as compared to D614 PSV NAb titers in human convalescent sera (right panel). Lines and bars represent the mean of the group and horizontal dotted lines the inverse of the lowest dilution tested.

FIG. 24 is a graph showing individual PsV NAb titers against variants of concern and SARS-COV-1 after a booster immunization in mRNA-primed (left panel) and subunit-primed (right panel) macaques.

### DETAILED DESCRIPTION OF THE INVENTION

The present disclosure provides immunogenic compositions that are protective against COVID-19. The compositions comprise a recombinant protein derived from the SARS-COV-2 S protein and expressed in a baculoviral/insect cell expression system. The recombinant protein may comprise an extracellular portion of the S protein (e.g., the entire or part of the S protein ectodomain), while lacking all or part of the transmembrane and cytoplasmic domains of the S protein. The recombinant protein may be comprised of three identical subunit polypeptides (i.e., a homotrimer), each containing a trimerization motif optimized for expression in a baculoviral/insect cell system that facilitates the trimerization of the three subunit polypeptides in a stabilized native prefusion trimer configuration. The immunogenic compositions further comprise a tocopherol-containing squalene emulsion adjuvant.

The immunogenic compositions herein can be used for prevention of symptomatic COVID-19 in SARS-COV-2 naïve human subjects, prevention of moderate-to-severe COVID-19 (e.g., prevention of hospitalization), prevention of asymptomatic infection, elicit immunogenicity against homologous matched strain, reduction in viral burden, and/or protection against circulating variant strains. Unless otherwise indicated, a SARS-COV-2 “variant” refers to a SARS-COV-2 strain that has one or more amino acid differences in the S protein from the original Wuhan strain (SEQ ID NO:1).

As used herein, the terms “immunogenic composition,” vaccine,” and “vaccine composition” are interchangeable and refer to a composition containing components that can elicit prophylactic protection against SARS-COV-2 infections, including alleviating COVID-19 symptoms and improving recovery and survival from the disease.

As used herein, percent identity between two amino acid sequences refers to the percentage of amino acid residues in the query sequence that are identical to the residues in the reference sequence, when the query and reference sequences are aligned for maximal identity. The homologous sequence may have the same length as the reference sequence or shorter (e.g., having at least 90% (e.g., at least 91, 92, 93, 94, 95, 96, 97, 98, or 99%) of the length of the reference sequence).

### I. Antigen Components of the Immunogenic Compositions

The immunogenic compositions of the present disclosure comprise a recombinant SARS-COV-2 S protein. The recombinant protein is stabilized to maintain the native, prefusion trimeric conformation on the viral envelope.

The SARS-COV-2 S protein has 1273 amino acid residues. An amino acid sequence of the S protein is available under NCBI Accession No. YP_009724390. The sequence is shown below. The signal sequence is boxed (MFVFLVLLPLVSS (SEQ ID NO:2)), and the transmembrane and intracellular domains are underlined. The S1 and S2 junction is between residues 685 and 686, which are in boldface and underlined.

| (SEQ ID NO: 1) | |
|---|---|
| 1 | 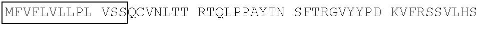 |
| 51 | TQDLFLPFFS NVTWFHAIHV SGTNGTKRFD NPVLPFNDGV YFASTEKSNI |
| 101 | IRGWIFGTTL DSKTQSLLIV NNATNVVIKV CEFQFCNDPF LGVYYHKNNK |
| 151 | SWMESEFRVY SSANNCTFEY VSQPFLMDLE GKQGNFKNLR EFVFKNIDGY |
| 201 | FKIYSKHTPI NLVRDLPQGF SALEPLVDLP IGINITRFQT LLALHRSYLT |
| 251 | PGDSSSGWTA GAAAYYVGYL QPRTFLLKYN ENGTITDAVD CALDPLSETK |
| 301 | CTLKSFTVEK GIYQTSNFRV QPTESIVRFP NITNLCPFGE VFNATRFASV |
| 351 | YAWNRKRISN CVADYSVLYN SASFSTFKCY GVSPTKLNDL CFTNVYADSF |
| 401 | VIRGDEVRQI APGQTGKIAD YNYKLPDDFT GCVIAWNSNN LDSKVGGNYN |
| 451 | YLYRLFRKSN LKPFERDIST EIYQAGSTPC NGVEGFNCYF PLQSYGFQPT |
| 501 | NGVGYQPYRV VVLSFELLHA PATVCGPKKS TNLVKNKCVN FNFNGLTGTG |
| 551 | VLTESNKKFL PFQQFGRDIA DTTDAVRDPQ TLEILDITPC SFGGVSVITP |
| 601 | GTNTSNQVAV LYQDVNCTEV PVAIHADQLT PTWRVYSTGS NVFQTRAGCL |
| 651 | IGAEHVNNSY ECDIPIGAGI CASYQTQTNS PRRARSVASQ SIIAYTMSLG |
| 701 | AENSVAYSNN SIAIPTNFTI SVTTEILPVS MTKTSVDCTM YICGDSTECS |
| 751 | NLLLQYGSFC TQLNRALTGI AVEQDKNTQE VFAQVKQIYK TPPIKDFGGF |
| 801 | NFSQILPDPS KPSKRSFIED LLFNKVTLAD AGFIKQYGDC LGDIAARDLI |
| 851 | CAQKFNGLTV LPPLLTDEMI AQYTSALLAG TITSGWTFGA GAALQIPFAM |
| 901 | QMAYRFNGIG VTQNVLYENQ KLIANQFNSA IGKIQDSLSS TASALGKLQD |
| 951 | VVNQNAQALN TLVKQLSSNF GAISSVLNDI LSRLDKVEAE VQIDRLITGR |
| 1001 | LQSLQTYVTQ QLIRAAEIRA SANLAATKMS ECVLGQSKRV DFCGKGYHLM |
| 1051 | SFPQSAPHGV VFLHVTYVPA QEKNFTTAPA ICHDGKAHFP REGVFVSNGT |
| 1101 | HWFVTQRNFY EPQIITTDNT FVSGNCDVVI GIVNNTVYDP LQPELDSFKE |
| 1151 | ELDKYFKNHT SPDVDLGDIS GINASVVNIQ KEIDRLNEVA KNLNESLIDL |
| 1201 | QELGKYEQYI KWPWYIWLGF IAGLIAIVMV TIMLCCMTSC CSCLKGCCSC |
| 1251 | GSCCKFDEDD SEPVLKGVKL HYT |

The recombinant S protein herein is comprised of three identical polypeptides (“recombinant S polypeptide” herein). Prior to maturation, each recombinant S polypeptide may comprise a signal sequence suitable for protein expression in insect cells. For example, the signal sequence is derived from an insect or baculoviral protein. The signal sequence may also be an artificial signal sequence. In some embodiments, the signal sequence is derived from an insect or baculoviral protein, such as chitinase and GP64. An exemplary chitinase signal sequence is a wildtype chitinase signal sequence

(SEQ ID NO: 11)
MLYKLLNVLW LVAVSNA

or a mutant chitinase signal sequence

(SEQ ID NO: 3)
MPLYKLLNVL WLVAVSNA

A sequence homologous to this chitinase signal sequence (e.g., at least 95, 96, 97, 98, or 99% identical) may also be used, so long as the signal peptide function is retained. See also U.S. Pat. No. 8,541,003.

The recombinant S protein herein comprises a SARS-COV-2 S protein ectodomain sequence, e.g., the sequence that corresponds to residues 14 to 1,211 of SEQ ID NO:1. An exemplary SARS-COV-2 S protein ectodomain sequence is shown as follows:

(SEQ ID NO: 4)
QCVNLTTRTQ LPPAYTNSFT RGVYYPDKVF RSSVLHSTQD LFLPFFSNVT
WFHAIHVSGT NGTKRFDNPV LPFNDGVYFA STEKSNIIRG WIFGTTLDSK
TQSLLIVNNA TNVVIKVCEF QFCNDPFLGV YYHKNNKSWM ESEFRVYSSA
NNCTFEYVSQ PFLMDLEGKQ GNFKNLREFV FKNIDGYFKI YSKHTPINLV
RDLPQGFSAL EPLVDLPIGI NITRFQTLLA LHRSYLTPGD SSSGWTAGAA
AYYVGYLQPR TFLLKYNENG TITDAVDCAL DPLSETKCTL KSFTVEKGIY
QTSNFRVQPT ESIVRFPNIT NLCPFGEVFN ATRFASVYAW NRKRISNCVA
DYSVLYNSAS FSTFKCYGVS PTKLNDLCFT NVYADSFVIR GDEVRQIAPG
QTGKIADYNY KLPDDFTGCV IAWNSNNLDS KVGGNYNYLY RLFRKSNLKP
FERDISTEIY QAGSTPCNGV EGFNCYFPLQ SYGFQPTNGV GYQPYRVVVL
SFELLHAPAT VCGPKKSTNL VKNKCVNFNF NGLTGTGVLT ESNKKFLPFQ
QFGRDIADTT DAVRDPQTLE ILDITPCSFG GVSVITPGTN TSNQVAVLYQ
DVNCTEVPVA IHADQLTPTW RVYSTGSNVF QTRAGCLIGA EHVNNSYECD
IPIGAGICAS YQTQTNSPRR ARSVASQSII AYTMSLGAEN SVAYSNNSIA
IPTNFTISVT TEILPVSMTK TSVDCTMYIC GDSTECSNLL LQYGSFCTQL
NRALTGIAVE QDKNTQEVFA QVKQIYKTPP IKDFGGFNFS QILPDPSKPS
KRSFIEDLLF NKVTLADAGF IKQYGDCLGD IAARDLICAQ KFNGLTVLPP
LLTDEMIAQY TSALLAGTIT SGWTFGAGAA LQIPFAMQMA YRFNGIGVTQ
NVLYENQKLI ANQFNSAIGK IQDSLSSTAS ALGKLQDVVN QNAQALNTLV
KQLSSNFGAI SSVLNDILSR LDKVEAEVQI DRLITGRLQS LQTYVTQQLI
RAAEIRASAN LAATKMSECV LGQSKRVDFC GKGYHLMSFP QSAPHGVVFL
HVTYVPAQEK NFTTAPAICH DGKAHFPREG VFVSNGTHWF VTQRNFYEPQ
IITTDNTFVS GNCDVVIGIV NNTVYDPLQP ELDSFKEELD KYFKNHTSPD
VDLGDISGIN ASVVNIQKEI DRLNEVAKNL NESLIDLQEL GKYEQYIK

In some embodiments, the recombinant S protein may comprise the sequence of SEQ ID NO:4 but for certain amino acid substitutions as further described herein, and is at least 99% (e.g., at least 99.5, 99.6, 99.7, 99.8, 99.9%) identical to SEQ ID NO:4. In further embodiments, the residues at positions 669-672 of SEQ ID NO:4 (in boldface) are changed to residues GSAS (SEQ ID NO:6) and/or the residues at positions 973 and 74 of SEQ ID NO:4 (underlined) are changed to residues PP.

In some embodiments, the recombinant S protein comprises one or more common mutations found in variants circulating in the COVID-19 pandemic. One such mutation is the D614G mutation (numbering according to SEQ ID NO:1) associated with a majority of current COVID-19 incidences around the world. Other mutations that may be included in the recombinant S protein may be one or more of W152C, K417T/N, N440K, V445I, G446A/S, L452R, Y453F, L455F. F456L, A475V, G476S, T478I/K/A, V483A/F/I, E484Q/K/D/A, F490S/L, Q493L/R, S494P/L, Y495N, G496L, P499H, N501Y, V503F/I, Y505W/H, Q506H/K, and P681H mutations (numbering according to SEQ ID NO:1). In some embodiments, the recombinant S protein may include one or more of the mutations N440K, T479I/K/A, and D614G.

In some embodiments, the recombinant S proteins comprises one or more mutations found in SARS-COV-2 variants, such as B.1.1.7 (British or Alpha variant: e.g., N501Y/P681H/deletion of H69/V70), B.1.351 (South African or Beta variant: e.g., K417N/E484K/N501Y), B1.617 (Indian or Delta variant: e.g., the L452R/E484Q mutations), P.I (Brazilian or Gamma variant: e.g., K417T/E484K/N501Y), and CAL.20C strain (aka. B.1.429; California or Epsilon variant: e.g., W152C/L452R).

The ectodomain sequence in the recombinant S protein may be modified to improve expression of the protein in host cells (e.g., insect cells) and stability of the produced protein. In some embodiments, the S ectodomain sequence contains a mutation that removes the proprotein convertase (PPC) motif (furin cleavage site) at the junction of the S1 subunit and the S2 subunit. For example, the sequence at the furin cleavage site, RRAR (SEQ ID NO:5: corresponding to residues 682-685 of SEQ ID NO: 1) is changed to GSAS (SEQ ID NO:6). Such mutations help preserve the prefusion conformation of the native S protein.

In some embodiments, the ectodomain sequence contains other mutations that help maintain the recombinant S protein in a more stable conformation so as to facilitate antigenic presentation of the prefusion epitopes that are more likely to lead to neutralizing responses. For example, amino acids corresponding to residues 986 and 987 of SEQ ID NO:1 (KV) are mutated to PP (see. e.g., Wrapp, supra; Kirchdoerfer et al., Sci Rep. (2018) 8:15701: Xiong, supra).

The recombinant S protein herein comprises a trimerization domain at the C-terminal region optimized for expression in a baculovirus/insect cell expression system, such that the S protein can assume a stabilized prefusion conformation of the native S protein. The foldon domain coding sequence may be inserted between the last codon and the stop codon of the S ectodomain coding sequence. In some embodiments, the trimerization domain is derived from the foldon domain of T4 phage fibritin (see. e.g., Meier et al., J Mol Biol. (2004) 344(4): 1051-69; WO 2018/081318). An exemplary foldon sequence is shown below: GYIPEAPRDG QAYVRKDGEW VELSTEL (SEQ ID NO: 7).

In some embodiments, the foldon sequence may be optimized to enhance expression of the recombinant protein in host cells. For example, to enhance expression of the recombinant protein in insect cells (e.g., Spodoptera cells), the sequence encoding the foldon sequence may be codon-optimized. The following shows the native coding sequence (top) and a codon-optimized version (bottom) for a foldon domain (nucleotide point mutations are marked by asterisks):

```
                    * * * * * * * *
ggt tat att cct gaa gct cca aga gat ggg caa gct tac gtt cgt
ggt tat ata cca gag gct cct aga gat ggc caa gca tac gtg cgc
G Y I P E A P R D G Q A Y V R
                    * * * * *** * *
aaa gat ggc gaa tgg gta ttc ctt tct acc ttt tta (SEQ ID NO: 8)
aaa gat ggt gaa tgg gtc ttt ctc agc aca ttc tta (SEQ ID NO: 9)
K D G E W V F H S T F T (SEQ ID NO: 7)
```

The recombinant S protein may comprise a tag (e.g., a His tag, a FLAG tag, an HA tag, a Myc tag, or V5 tag) to facilitate purification.

In some embodiments, the recombinant S protein may be a trimer of a polypeptide having the following sequence, but without the signal sequence once processed and assembled. In the sequence below, the signal sequence is underlined (residues 1-18), the foldon sequence is double underlined (residues 1217-1243), while mutations relative to the wildtype sequence (artificially introduced) are in boldface and underlined (residues 687-690 and 991-992). This protein is also termed “preS dTM” or “D614 preS dTM” herein.

| (SEQ ID NO:10) | |
|---|---|
| 1 | MPLYKLLNVL WLVAVSNAQC VNLTTRTQLP PAYTNSFTRG VYYPDKVFRS |
| 51 | SVLHSTQDLF LPFFSNVTWF HAIHVSGING TKRFDNPVLP FNDGVYFAST |
| 101 | EKSNIIRGWI FGTTLDSKTQ SLLIVNNATN VVIKVCEFQF CNDPFLGVYY |
| 151 | HKNNKSWMES EFRVYSSANN CTFEYVSQPF LMDLEGKQGN FKNLREFVFK |
| 201 | NIDGYFKIYS KHTPINLVRD LPQGFSALEP LVDLPIGINI TRFQTLLALH |
| 251 | RSYLTPGDSS SGWTAGAAAY YVGYLQPRTF LLKYNENGTI TDAVDCALDP |
| 301 | LSETKCTLKS FTVEKGIYQT SNFRVQPTES IVRFPNITNL CPFGEVFNAT |
| 351 | RFASVYAWNR KRISNCVADY SVLYNSASFS TFKCYGVSPT KLNDLCFTNV |
| 401 | YADSFVIRGD EVRQIAPGQT GKIADYNYKL PDDFTGCVIA WNSNNLDSKV |
| 451 | GGNYNYLYRL FRKSNLKPFE RDISTEIYQA GSTPCNGVEG FNCYFPLQSY |
| 501 | GFQPTNGVGY QPYRVVVLSF ELLHAPATVC GPKKSTNLVK NKCVNFNFNG |
| 551 | LTGTGVLTES NKKFLPFQQF GRDIADTTDA VRDPQTLEIL DITPCSFGGV |
| 601 | SVITPGTNTS NQVAVLYQDV NCTEVPVAIH ADQLTPTWRV YSTGSNVFQT |
| 651 | RAGCLIGAEH VNNSYECDIP IGAGICASYQ TQTNSPGSAS SVASQSIIAY |
| 701 | TMSLGAENSV AYSNNSIAIP TNFTISVTTE ILPVSMTKTS VDCTMYICGD |
| 751 | STECSNLLLQ YGSFCTQLNR ALTGIAVEQD KNTQEVFAQV KQIYKTPPIK |
| 801 | DFGGFNFSQI LPDPSKPSKR SFIEDLLFNK VTLADAGFIK QYGDCLGDIA |
| 851 | ARDLICAQKF NGLTVLPPLL TDEMIAQYTS ALLAGTITSG WTFGAGAALQ |
| 901 | IPFAMQMAYR FNGIGVTQNV LYENQKLIAN QFNSAIGKIQ DSLSSTASAL |
| 951 | GKLQDVVNQN AQALNTLVKQ LSSNFGAISS VLNDILSRLD PPEAEVQIDR |
| 1001 | LITGRLQSLQ TYVTQQLIRA AEIRASANLA ATKMSECVLG QSKRVDFCGK |
| 1051 | GYHLMSFPQS APHGVVFLHV TYVPAQEKNF TTAPAICHDG KAHFPREGVF |
| 1101 | VSNGTHWFVT QRNFYEPQII TTDNTFVSGN CDVVIGIVNN TVYDPLQPEL |
| 1151 | DSFKEELDKY FKNHTSPDVD LGDISGINAS VVNIQKEIDR LNEVAKNLNE |
| 1201 | SLIDLQELGK YEQYIKGYIP EAPRDGQAYV RKDGEWVFLS TFL |

A sequence homologous to SEQ ID NO: 10 may also be used. For example, a recombinant S polypeptide whose sequence is at least 95% (e.g., at least 96, 97, 98, or 99%) identical to SEQ ID NO:10 may be used. The homologous sequence may have the same length as SEQ ID NO: 10 or no more than 10% (e.g., no more than 9, 8, 7, 6, 5, 4, 3, 2, or 1%) shorter or longer than SEQ ID NO:10. In further embodiments, residues GSAS (SEQ ID NO:6) at positions 687-690 of SEQ ID NO: 10 and/or residues PP at positions 991 and 992 of SEQ ID NO: 10 are maintained in such a homologous sequence. The percent identity of two amino acid sequences may be obtained by, e.g., BLAST® using default parameters (available at the U.S. National Library of Medicine's National Center for Biotechnology Information website).

In some embodiments, a variant of preS dTM (also “preS dTM variant” herein), i.e., a recombinant S protein containing one or more amino acid differences from SEQ ID NO:10 (e.g., outside the signal sequence region), is used. In further embodiments, the recombinant S protein is derived from the Southern African or Beta variant B.1.351. This variant contains the following mutations (relative to the Wuhan strain or SEQ ID NO:1): (i) in the NTD domain: L18F, D80A, D215G, L242del, A243del, and L244del; (ii) in the RBD domain: K417N, E484K, N501Y; (iii) in the S1 domain: D614G; and (iv) A701V. The S protein may comprise the following sequence (SEQ ID NO:13), without the signal sequence (underlined; residues 1-18) once processed and secreted from producing cells. The T4 foldon sequence is double underlined (residues 1214-1240); variations from SEQ ID NO:10 are boxed and boldfaced; and artificially introduced mutations (residues 684-687 and residues 988-989) are underlined and boldfaced). Compared to the S protein derived from the Wuhan strain, this protein also has a deletion of three residues “LAL” immediately after “FQTL” at positions 243-246 below.

| (SEQ ID NO: 13) | |
|---|---|
| 1 | 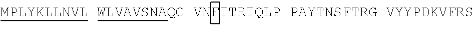 |
| 51 | 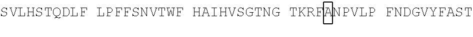 |
| 101 | EKSNIIRGWI FGTTLDSKTQ SLLIVNNATN VVIKVCEFQF CNDPFLGVYY |
| 151 | HKNNKSWMES EFRVYSSANN CTFEYVSQPF LMDLEGKQGN FKNLREFVFK |
| 201 | 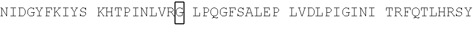 |
| 251 | LTPGDSSSGW TAGAAAYYVG YLQPRTFLLK YNENGTITDA VDCALDPLSE |
| 301 | TKCTLKSFTV EKGIYQTSNF RVQPTESIVR FPNITNLCPF GEVFNATRFA |
| 351 | SVYAWNRKRI SNCVADYSVL YNSASFSTFK CYGVSPTKLN DLCFTNVYAD |
| 401 | 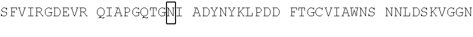 |
| 451 | 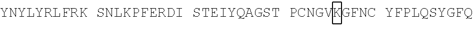 |
| 501 | 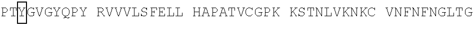 |
| 551 | TGVLTESNKK FLPFQQFGRD IADTTDAVRD PQTLEILDIT PCSFGGVSVI |
| 601 | 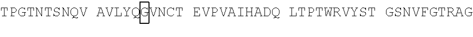 |
| 651 | CLIGAEHVNN SYECDIPIGA GICASYQTQT NSPGSASSVA SQSIIAYTMS |
| 701 | 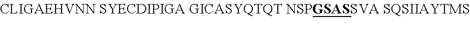 |
| 751 | CSNLLLQYGS FCTQLNRALT GIAVEQDKNT QEVFAQVKQI YKTPPIKDFG |
| 801 | GFNFSQILPD PSKPSKRSFI EDLLFNKVTL ADAGFIKQYG DCLGDIAARD |
| 851 | LICAQKFNGL TVLPPLLTDE MIAQYTSALL AGTITSGWTF GAGAALQIPF |
| 901 | AMQMAYRFNG IGVTQNVLYE NQKLIANQFN SAIGKIQDSL SSTASALGKL |
| 951 | QDVVNQNAQA LNTLVKQLSS NFGAISSVLN DILSRLDPPE AEVQIDRLIT |
| 1001 | GRLQSLQTYV TQQLIRAAEI RASANLAATK MSECVLGQSK RVDFCGKGYH |
| 1051 | LMSFPQSAPH GVVFLHVTYV PAQEKNFTTA PAICHDGKAH FPREGVFVSN |
| 1101 | GTHWFVTQRN FYEPQIITTD NTFVSGNCDV VIGIVNNTVY DPLQPELDSF |
| 1151 | KEELDKYFKN HTSPDVDLGD ISGINASVVN IQKEIDRLNE VAKNLNESLI |
| 1201 | DLQELGKYEQ YIKGYIPEAP RDGQAYVRKD GEWVFLSTFL |

In some embodiments, the present immunogenic composition is multivalent (e.g., bivalent, trivalent, or quadrivalent). That is, the composition comprises multiple (e.g., two, three, or four) different recombinant S proteins. One or more of the recombinant S proteins in a multivalent composition may comprise one or more mutations found in SARS-COV-2 variants, such as D614G and mutations found in newly emergent variant strains, e.g., B.1.1.7, B.1.351, B.1.617, P.1, and CAL.20C.

In some embodiments, the present immunogenic composition is bivalent. In further embodiments, the bivalent composition comprises a first recombinant S protein that is derived from the Wuhan strain and a second recombinant S protein that is derived from the South African strain. In certain embodiments, the bivalent composition comprises a recombinant S protein comprising SEQ ID NO:10, without the signal sequence, and a recombinant S protein comprising SEQ ID NO: 13, without the signal sequence.

### II. Adjuvant Components of the Immunogenic Compositions

The present immunogenic compositions comprise tocopherol-containing, squalene-based oil-in-water (O/W) emulsion adjuvants having pharmaceutically acceptable ingredients. Such adjuvants, also referred to as tocopherol-containing squalene emulsion adjuvants, enhance the magnitude and/or quality of the immune response to the recombinant S protein.

Squalene is a branched, unsaturated terpenoid with the chemical formula [(CH₃)₂C[═CHCH₂CH₂C(CH₃)]₂═CHCH₂-]₂ (i.e., C₃₀H₅₀; CAS Registry Number 7683-64-9). It is also known as 2,6,10,15,19,23-hexamethyl-2,6,10,14,18,22-tetracosahexaene. Squalene shows good biocompatibility and is readily metabolized.

The tocopherol-containing squalene emulsion adjuvants contain one or more tocopherols. Any of the α, β, γ, δ, ε and/or ξ tocopherols can be used, but α-tocopherol (or alpha-tocopherol) is typically used. D-alpha-tocopherol and D/L-alpha-tocopherol can both be used. In some embodiments, the tocopherol-containing squalene emulsion adjuvant contains alpha-tocopherol, for example, D/L-alpha-tocopherol.

Tocopherol-containing squalene emulsion adjuvants will typically have a submicron (less than 1 μm) droplet size. In some embodiments, the droplet on average is less than 500 nm, e.g., less than 200 nm. Droplet sizes below 200 nm are beneficial in that they can facilitate sterilization by filtration. There is evidence that droplet sizes in the 80 to 200 nm range are of interest for potency, manufacturing consistency and stability reasons (Klucker et al., J Pharm Sci. (2012) 101(12):4490-500; Shah et al., Nanomedicine (Lond) (2014) 9:2671-81: Shah et al., J Pharm Sci. (2015) 104:1352-61: Shah et al., Scientific Reports (2019) 9:11520). In some embodiments, the adjuvant has an average droplet size of at least 50 nm, at least 80 nm, or at least 100 nm (e.g., at least 120 nm). For example, the adjuvant may have an average droplet size of 50 to 200 nm (e.g., 80 to 200 nm), 120 to 180 nm, or 140 to 180 nm, such as about 160 nm.

Droplet size uniformity is desirable. A polydispersity index (PdI) of greater than 0.7 indicates that the sample has a very broad size distribution and a reported value of 0) means that size variation is absent. Suitably the tocopherol-containing squalene emulsion adjuvant has a PdI of 0.5 or less, or 0.3 or less, such as 0.2 or less.

The droplet size, as used herein, means the average diameter of oil droplets in an emulsion and can be determined in various ways, e.g., using the techniques of dynamic light scattering and/or single-particle optical sensing with an apparatus such as the Accusizer™ and Nicomp™ series of instruments available from Particle Sizing Systems (Santa Barbara, USA), the Zetasizer™ instruments from Malvern Instruments (UK), or the Particle Size Distribution Analyzer instruments from Horiba (Kyoto, Japan) (Schartl, Light Scattering from Polymer Solutions and Nanoparticle Dispersions (2007)). Dynamic light scattering (DLS) is a method by which droplet size is determined. A method for defining the average droplet diameter is a Z-average, i.e., the intensity-weighted mean hydrodynamic size of the ensemble collection of droplets measured by DLS. The Z-average is derived from cumulants analysis of the measured correlation curve, wherein a single particle size (droplet diameter) is assumed and a single exponential fit is applied to the autocorrelation function. Thus, references herein to average droplet size should be taken as an intensity-weighted average, and ideally the Z-average. PdI values are easily provided by the same instrumentation which measures average diameter.

In order to maintain a stable submicron emulsion, one or more emulsifying agents (i.e., surfactants) are generally required. Surfactants can be classified by their “HLB” (Griffin's hydrophile/lipophile balance), where an HLB in the range 1-10 generally means that the surfactant is more soluble in oil than in water, whereas an HLB in the range 10-20 means that the surfactant is more soluble in water than in oil. HLB values are readily available for many surfactants of interest or can be determined experimentally, e.g., polysorbate 80 has an HLB of 15.0 and TPGS has an HLB of 13 to 13.2. Sorbitan trioleate has an HLB of 1.8. When two or more surfactants are blended, the resulting HLB of the blend is typically calculated by the weighted average. For example, a 70/30 weight % mixture of polysorbate 80 and TPGS has an HLB of (15.0×0.70)+(13×0.30), i.e., 14.4. A 70/30 weight % mixture of polysorbate 80 and sorbitan trioleate has a HLB of (15.0×0.70)+(1.8×0.30), i.e., 11.04.

Surfactant(s) will typically be metabolizable (biodegradable) and biocompatible, being suitable for use as a pharmaceutical. The surfactant can include ionic (cationic, anionic or zwitterionic) and/or nonionic surfactants. The use of only nonionic surfactants is desirable due to, for example, their pH independence. The invention can thus use surfactants including, but not limited to: (i) the polyoxyethylene sorbitan ester surfactants (commonly referred to as the Tweens or polysorbates), such as polysorbate 20 and polysorbate 80; (ii) copolymers of ethylene oxide (EO), propylene oxide (PO), and/or butylene oxide (BO), sold under the DOWFAX™, Pluronic™ (e.g., F68, F127 or L121 grades) or Synperonic™ tradenames, such as linear EO/PO block copolymers, for example poloxamer 407, poloxamer 401 and poloxamer 188: (iii) octoxynols, which can vary in the number of repeating ethoxy (oxy-1,2-ethanediyl) groups, with octoxynol-9) (Triton X 100, or t-octylphenoxypolyethoxyethanol) being of interest: (iv) (octylphenoxy)polyethoxyethanol (IGEPAL CA-630/NP-40): (v) phospholipids such as phosphatidylcholine (lecithin): (vi) polyoxyethylene fatty ethers derived from lauryl, cetyl, stearyl and oleyl alcohols (known as Brij R: surfactants), such as polyoxyethylene-4-lauryl ether (Brij R: 30, Emulgin R: 104P), polyoxyethylene-9-lauryl ether and polyoxyethylene 12 cetyl/stearyl ether (Eumulgin R: B 1, cetereth-12 or polyoxyethylene cetostearyl ether): (vii) sorbitan esters (commonly known as the Spans), such as sorbitan trioleate (Span R: 85), sorbitan monooleate (Span R: 80) and sorbitan monolaurate (Span R: 20): or (viii) tocopherol derivative surfactants, such as alpha-tocopherol-polyethylene glycol succinate (TPGS). Many examples of pharmaceutically acceptable surfactants are known in the art. See. e.g., Handbook of Pharmaceutical Excipients (2009 6^{th} ed.). Methods for optimizing the choice of surfactant used in a squalene emulsion adjuvant are illustrated in Klucker et al., J Pharm Sci. (2012) 101(12):4490-500.

In general, the surfactant component has an HLB between 10 and 18, such as between 12 and 17 (e.g., 13 to 16). This can be typically achieved using a single surfactant or, in some embodiments, using a mixture of surfactants. Surfactants of interest may include: poloxamer 401, poloxamer 188, polysorbate 80, sorbitan trioleate, sorbitan monooleate and polyoxyethylene 12 cetyl/stearyl ether either alone, in combination with each other or in combination with other surfactants. Of interest are polysorbate 80, sorbitan trioleate, sorbitan monooleate and polyoxyethylene 12 cetyl/stearyl ether either alone, or in combination with each other. A surfactant of interest is polysorbate 80. A combination of surfactants of interest is polysorbate 80 and sorbitan trioleate. A further combination of surfactants of interest is sorbitan monooleate and polyoxyethylene cetostearyl ether.

In certain embodiments, the tocopherol-containing squalene emulsion adjuvant comprises one surfactant, such as polysorbate 80. In some embodiments, the tocopherol-containing squalene emulsion adjuvant comprises two surfactants, such as polysorbate 80 and sorbitan trioleate or sorbitan monooleate and polyoxyethylene cetostearyl ether. In other embodiments, the tocopherol-containing squalene emulsion adjuvant comprises three or more surfactants, such as three surfactants.

Desirably, the weight ratio of squalene to tocopherol is 20 or less (i.e., 20 weight units of squalene or less per weight unit of tocopherol or, alternatively phrased, at least 1 weight unit of tocopherol per 20 weight units of squalene), such as 10 or less. Suitably the weight ratio of squalene to tocopherol is 0.1 or more. Typically, the weight ratio of squalene to tocopherol is 0.1 to 10, 0.2 to 5, or 0.3 to 3, such as 0.4 to 2. Suitably, the weight ratio of squalene to tocopherol is 0.72 to 1.136, 0.8 to 1, or 0.85 to 0.95, such as 0.9.

Typically, the weight ratio of squalene to surfactant is 0.73 to 6.6, 1 to 5, or 1.2 to 4. Suitably, the weight ratio of squalene to surfactant is 1.71 to 2.8, 2 to 2.4, or 2.1 to 2.3, such as 2.2.

The amount of squalene in a single dose, such as a single human dose, of tocopherol-containing squalene emulsion adjuvant is typically at least 1.2 mg. Generally, the amount of squalene in a single dose, such as a single human dose, of tocopherol-containing squalene emulsion adjuvant is 50 mg or less. The amount of squalene in a single dose, such as a single human dose, of tocopherol-containing squalene emulsion adjuvant may be 1.2 to 20 mg (e.g., 1.2 to 15 mg). The amount of squalene in a single dose, such as a single human dose, of tocopherol-containing squalene emulsion adjuvant may be 1.2 to 2 mg. 2 to 4 mg. 4 to 8 mg or 8 to 12.1 mg. For example, the amount of squalene in a single dose, such as a single human dose, of tocopherol-containing-squalene emulsion adjuvant may be 1.21 to 1.52 mg. 2.43 to 3.03 mg, 4.87 to 6.05 mg, or 9.75 to 12.1 mg.

The amount of tocopherol in a single dose, such as a single human dose, of tocopherol-containing-squalene emulsion adjuvant is typically at least 1.3 mg. Generally, the amount of tocopherol in a single dose, such as a single human dose, of tocopherol-containing squalene emulsion adjuvant is 55 mg or less. The amount of tocopherol in a single dose, such as a single human dose, of tocopherol-containing squalene emulsion adjuvant may be 1.3 to 22 mg (e.g., 1.3 to 16.6 mg). The amount of tocopherol in a single dose, such as a single human dose, of tocopherol-containing squalene emulsion adjuvant may be 1.3 to 2 mg, 2 to 4 mg, 4 to 8 mg or 8 to 13.6 mg. For example, the amount of tocopherol in a single dose, such as a single human dose, of tocopherol-containing squalene emulsion adjuvant may be 1.33 to 1.69 mg. 2.66 to 3.39 mg, 5.32 to 6.77 mg, or 10.65 to 13.53 mg.

The amount of surfactant in a single dose, such as a single human dose, of tocopherol-containing-squalene emulsion adjuvant is typically at least 0.4 mg. Generally, the amount of surfactant in a single dose, such as a single human dose, of tocopherol-containing squalene emulsion adjuvant is 18 mg or less. The amount of surfactant in a single dose, such as a single human dose, of tocopherol-containing squalene emulsion adjuvant may be 0.4 to 9.5 mg (e.g., 0.4 to 7 mg). The amount of surfactant in a single dose, such as a single human dose, of tocopherol-containing squalene emulsion adjuvant may be 0.4 to 1 mg. 1 to 2 mg, 2 to 4 mg or 4 to 7 mg. For example, the amount of surfactant in a single dose, such as a single human dose, of tocopherol-containing squalene emulsion adjuvant may be 0.54 to 0.71 mg. 1.08 to 1.42 mg, 2.16 to 2.84 mg, or 4.32 to 5.68 mg.

In certain embodiments the tocopherol-containing squalene emulsion adjuvant may comprise or consist essentially of squalene, tocopherol, surfactant, and water. For example, in addition to squalene, tocopherol, surfactant and water, tocopherol-containing squalene emulsion adjuvants may contain additional components as desired or required depending upon the intended final presentation and vaccination strategy, such as buffers and/or tonicity modifying agents, for example, modified phosphate-buffered saline (disodium phosphate, potassium biphosphate, sodium chloride, and potassium chloride).

High pressure homogenization (HPH or microfluidization) may be applied to yield tocopherol-containing squalene emulsion adjuvants with uniformly small droplet sizes and long-term stability (see. e.g., EP 0868918B1 and WO2006/100109). Briefly, oil phase composed of squalene and tocopherol may be formulated under a nitrogen atmosphere. Aqueous phase is prepared separately, typically composed of water for injection or phosphate-buffered saline, and polysorbate 80. Oil and aqueous phases are combined, such as at a ratio of 1:9 (volume of oil phase to volume of aqueous phase) before homogenization and microfluidization, such as by a single pass through an in-line homogenizer and three passes through a microfluidizer (at around 15000 psi). The resulting emulsion may then be sterile filtered, for example, through two trains of two 0.5/0.2 μm filters in series (i.e., 0.5/0.2/0.5/0.2) (see. e.g., WO2011/154444). Operation is desirably undertaken under an inert atmosphere, e.g., nitrogen. Positive pressure may be applied (see. e.g., WO2011/154443).

International patent application WO2020/160080 and Lodaya et al. (J Control Release (2019) 316:12-21) describe tocopherol-containing squalene emulsion adjuvants that are self-emulsifying adjuvant systems (SEAS) and their manufacture.

In some embodiments, the tocopherol-containing squalene emulsion adjuvant is the AS03 adjuvant. See. e.g., WO2006/100109; Garçon et al., Expert Rev Vaccines (2012) 11:349-66; Cohet et al., Vaccine (2019) 37(23):3006-21. This adjuvant includes squalene, alpha-tocopherol and polysorbate 80. For adult human use, a single full dose of the adjuvant (also called AS03_{A}) is 0.25 mL of an oil-in-water emulsion containing 10.69 mg squalene, 11.86 mg alpha-tocopherol and 4.86 mg polysorbate 80 and PBS (Fox, Molecules (2009) 14:3286-312: Morel et al., Vaccine (2011) 29:2461-73). See also Table 9 below.

Certain reduced doses of AS03 have also been described (WO2008/043774), including AS03_{B} (½ dose), AS03_{C} (¼ dose) and AS03_{D} (⅛ dose) (Carmona Martinez et al., Hum Vaccin Immunother. (2014) 10(7): 1959-68). Thus, where desired, the oil-in-water emulsion adjuvant contains only ½, ¼, or ⅛ of the amounts of squalene, alpha-tocopherol, and polysorbate 80 per (single) dose as AS03_{A}. These reduced doses, e.g. AS03_{B} or AS03_{C}, may be useful when a reduced reactogenicity is desirable, for example, in pediatric subjects. For example, a single adjuvant dose may contain: (i) 5.34 mg squalene, 5.93 mg alpha-tocopherol, and 2.43 mg polysorbate 80 (e.g., AS03_{B}: 125 μL of the oil-in-water emulsion shown in Table 9 below): (ii) 2.67 mg squalene, 2.97 mg alpha-tocopherol and 1.22 mg polysorbate 80 (e.g., AS03_{C}: 62.5 μL of the oil-in-water emulsion shown in Table 9 below); or (iii) 1.34 mg squalene, 1.48 mg alpha-tocopherol and 0.61 mg polysorbate 80 (e.g., AS03_{D}: i.e., 31.25 μL of the oil-in-water emulsion shown in Table 9 below). Typically, the final volume of a single dose of the AS03 adjuvant is 0.25 mL or 0.5 mL. Therefore, if the volume of a concentrated oil-in-water emulsion bulk (e.g., the oil-in-water emulsion of Table 9 below) needed to match the above desired amounts of squalene, alpha-tocopherol and polysorbate 80 is below 0.25 mL or 0.5 mL, such volume may be made up to the desired volume (0.25 mL or 0.5 mL) with a phosphate-buffered saline.

To limit undesired degradation, tocopherol-containing squalene emulsions should generally be stored with limited exposure to oxygen e.g. in containers with limited headspace and/or by storage under nitrogen.

A potential safety issue with coronavirus vaccines is the ability to potentiate immunopathology from vaccines upon exposure to wild-type virus (Smatti et al., Front Microbiol. (2018) 9:2991). The molecular mechanism for this phenomenon, termed antibody-dependent enhancement or immune enhancement of viral infection, is still not fully understood. In the context of coronavirus infections, various factors have been suggested as potentially contributing to the phenomenon. These include the epitope targeted, the method of delivery of the antigen, the magnitude of the immune responses, the balance between binding and functional antibodies, the elicitation of antibodies with functional characteristics such as binding to particular Fc receptors, and the nature of the T-helper cell response (Tseng et al., PLOS One (2012) 7(4): Yasui et al., J Immunol. (2008) 181(9):6337-48: Czub et al., Vaccine (2005) 23(17-18):2273-9). The inclusion of adjuvanted formulations including adjuvants such as AS03 is anticipated to further enhance the magnitude of neutralizing antibody responses and thereby alleviate antibody-dependent enhancement of viral infection, which is thought to be mediated mostly by non-neutralizing antibodies.

### III. Production of Recombinant S Protein

The viral antigen component of the present immunogenic compositions may be produced by recombinant technology in insect cells (e.g., Drosophila S2 cells, Spodoptera frugiperda cells, Sf9 cells, Sf21, High Five cells, or expresSF+ cells) that have been transduced with a baculoviral expression vector, such as one derived from Autographa californica multiple nucleopolyhedrovirus (AcMNPV). Baculoviruses such as AcMNPV form large protein crystalline occlusions within the nucleus of infected cells, with a single polypeptide termed polyhedrin accounting for approximately 95% of the protein mass. The gene for polyhedrin is present as a single copy in the baculoviral genome and can be readily replaced with foreign genes because it is not essential for virus replication in cultured cells. Recombinant baculoviruses that express a foreign gene such as the recombinant S polypeptide are constructed by way of homologous recombination between baculovirus genomic DNA and a transfer plasmid containing the foreign gene.

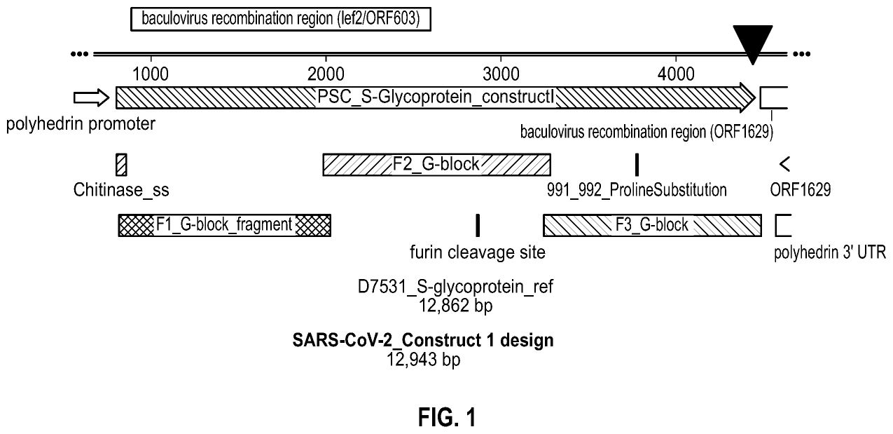

In certain embodiments, the transfer plasmid contains an expression cassette for the recombinant S polypeptide, where the expression cassette is flanked by sequences naturally flanking the polyhedrin locus in the AcMNPV (FIG. 1). The transfer plasmid is co-transfected into host cells with baculovirus genomic DNA that has been linearized with an enzyme (e.g., Bsu36I) that removes the polyhedrin gene and a part of an essential gene downstream of the polyhedrin locus so that parental viral DNA molecule cannot replicate, rendering the genomic DNA non-infectious; however, this part of the essential gene is present on the transfer plasmid. After co-transfection, homologous recombination between the transfer plasmid and the linearized genomic DNA recircularizes the genomic viral DNA, restoring its ability to replicate. Because the original baculovirus genomic DNA before linearization contains the polyhedrin gene, plaques formed by non-recombinant virus are cloudy (due to the crystalline occlusions in the infected cells), whereas plaques formed by recombinant virus are clear.

The baculoviral expression vector may be engineered to increase the yield of the recombinant protein. In some embodiments, the baculoviral vector has one or more genes knocked out. The baculovirus genome contains genes that are non-essential for virus replication in cell culture and for expression of recombinant proteins. Deletion of such genes may remove unnecessary genetic burden, help generate more stable baculoviral expression vectors, reduce time needed for established insect cell infection, and result in more efficient expression of the recombinant protein. In some embodiments, the polyhedrin promoter is modified by including in it more than one copy of the burst sequence: for example, the promoter may be engineered to include two burst sequences to create a “double burst” (DB) promoter, which contains two repeats of the nucleotide sequence CTGTTTTCGTAACAGTTTTGTAATAAAAAAACCTATAAATA (SEQ ID NO:12). See. e.g., Manohar et al., Biotechnol Bioeng. (2010) 107:909-16. To integrate the viral antigen coding sequence into a baculoviral expression vector, a transfer plasmid carrying the coding sequence may be integrated to the DNA encoding the baculoviral genome through homologous recombination. Viral identity may be confirmed by, for example, Southern blot or Sanger sequencing analysis of the S protein coding sequence insert from purified baculovirus DNA and Western blot analyses of the recombinant protein produced in infected insect cells. See, e.g., U.S. Pat. Nos. 6,245,532 and 8,541,003.

Host cells containing the viral antigen expression construct are cultured in bioreactors (e.g., 45L, 60L, 459L, 2000L, or 20,000L) in, e.g., a batch process or a fed-batch process. The produced S protein may be isolated from the cell cultures by, for example, column chromatography in either flow-through or bind-and-elute modes. Examples are ion exchange resins and affinity resins, such as lentil lectin Sepharose, and mixed mode cation exchange-hydrophobic interaction columns (CEX-HIC). The protein may be concentrated, buffer exchanged by ultrafiltration, and the retentate from the ultrafiltration may be filtered through a 0.22 μm filter. See. e.g., McPherson et al., “Development of a SARS Coronavirus Vaccine from Recombinant Spike Protein Plus Delta Inulin Adjuvant,” Chapter 4, in Sunil Thomas (ed.), Vaccine Design: Methods and Protocols: Volume 1: Vaccines for Human Diseases, Methods in Molecular Biology, Springer, New York, 2016. See also U.S. Pat. 5.762,939.

The baculovirus expression vector system (BEVS) provides an excellent method for the development of the ideal subunit vaccine. Recombinant protein can be produced by such systems in approximately eight weeks. Speedy production is especially critical when there is a pandemic threat. Further, baculoviruses are safe by virtue of their narrow host range, which is restricted to a few taxonomically related insect species, and have not been observed to replicate in mammalian cells. Additionally, very few microorganisms are known to be able to replicate in both insect cells and mammalian cells: thus, the possibility of adventitious agent contamination in clinical products made by insect cells is very low. Moreover, humans generally do not have pre-existing immunity to proteins from insects that are the natural hosts for baculoviruses, because these insects are non-biting: thus, allergic reactions to clinical products made in BEV systems are not likely. Further, although the carbohydrate moieties added to proteins in insect cells appear to be less complex than those on their mammalian cell-expressed counterparts, the immunogenicity of insect cell-expressed and mammalian cell-expressed glycoproteins appear to be equivalent. Full-length proteins expressed in baculovirus systems usually self-assemble into the higher-order structures normally assumed by the natural proteins by modulating the surfactant concentration. Finally, the BEVS system is highly efficient due to the extremely high activity of the polyhedrin promoter, which allows production of recombinant protein at high levels at significantly lower costs.

### IV. Formulation and Packaging of Vaccines

The recombinant S protein(s) and tocopherol-containing squalene emulsion adjuvant may be administered via various suitable routes, including parenteral, such as intramuscular or subcutaneous administration. The immunogenic composition may be monovalent or multivalent as described above. Suitably the recombinant S protein and tocopherol-containing squalene emulsion adjuvant are formulated for intramuscular (IM) injection. The recombinant S protein and tocopherol-containing squalene emulsion adjuvant may be administered to a subject in a mixture, e.g., through IM injection in the subject's deltoid muscle in the upper arm. Alternatively, the recombinant S protein and tocopherol-containing squalene emulsion adjuvant may be administered separately through the same or different routes, to the same or different locations, and at the same or different times.

When administered as separate formulations, the recombinant S protein and tocopherol-containing squalene emulsion adjuvant are desirably administered to locations with sufficient spatial proximity such that the adjuvant effect is adequately maintained. For example, spatial proximity is sufficient to maintain at least 50%, at least 75%, or at least 90% of the adjuvant effect seen with administration at to the same location. The adjuvant effect seen with administration to the same location is defined as the level of increase observed as a result of administration of recombinant S protein and tocopherol-containing squalene emulsion adjuvant to the same location compared with administration of recombinant S protein alone. The recombinant S protein and tocopherol-containing squalene emulsion adjuvant are desirably administered to a location draining to the same lymph node, such as to the same limb or to the same muscle. Suitably recombinant S protein and tocopherol-containing squalene emulsion adjuvant are administered intramuscularly to the same muscle. In certain embodiments, the recombinant S protein and tocopherol-containing squalene emulsion adjuvant are administered to the same location. The spatial separation of administration locations may be at least 5 mm, such as at least 1 cm. The spatial separation of administration locations may be less than 10 cm, such as less than 5 cm apart.

When administered as separate formulations, the recombinant S protein and tocopherol-containing squalene emulsion adjuvant are desirably administered with sufficient temporal proximity such that the adjuvant effect is adequately maintained. For example, temporal proximity is sufficient to maintain at least 50%, at least 75%, or at least 90% of the adjuvant effect seen with administration at the same time. The adjuvant effect seen with administration at the same time is defined as the level of increase observed as a result of administration at (essentially) the same time compared with administration of recombinant S protein without tocopherol-containing squalene emulsion adjuvant. When administered as separate formulations, recombinant S protein and tocopherol-containing squalene emulsion adjuvant may be administered within 12 hours. Suitably the recombinant S protein and tocopherol-containing squalene emulsion adjuvant are administered within 6 hours, within 2 hours, or within 1 hour, such as within 30 minutes or within 15 minutes (e.g., within 5 minutes). The delay between administration of the recombinant S protein and tocopherol-containing squalene emulsion adjuvant may be at least 5 seconds (e.g., 10) seconds) or at least 30 seconds. When administered as separate formulations, if the recombinant S protein and tocopherol-containing squalene emulsion adjuvant are administered with a delay, the recombinant S protein may be administered first and the tocopherol-containing squalene emulsion adjuvant administered second. Alternatively, the tocopherol-containing squalene emulsion adjuvant is administered first and the recombinant S protein administered second. Appropriate temporal proximity may depend on the order or administration. Desirably, the recombinant S protein and tocopherol-containing squalene emulsion adjuvant are administered without intentional delay (accounting for the practicalities of multiple administrations).

In addition to co-formulated or separately formulated presentations of recombinant S protein and tocopherol-containing squalene emulsion adjuvant for direct administration, the recombinant S protein and tocopherol-containing squalene emulsion adjuvant may initially be provided in various forms which facilitate manufacture, storage and distribution. For example, certain components may have limited stability in liquid form, certain components may not be amendable to drying, certain components may be incompatible when mixed (either on a short- or long-term basis). Independent of whether recombinant S protein and tocopherol-containing squalene emulsion are co-formulated at administration, they may be provided in separate containers the contents of which are subsequently combined. The recombinant S protein may be provided in liquid or dry (e.g. lyophilized) form: the chosen form will depend on factors such as the precise nature of the recombinant S protein, e.g., if the recombinant S protein is amenable to drying, or other components which may be present. The tocopherol-containing squalene emulsion adjuvant is typically provided in liquid form.

The immunogenic composition may be in the form of an extemporaneous formulation, where the antigen and the adjuvant are brought into contact just before or at the time of use. For example, the antigen can be mixed volume to volume with the adjuvant (emulsion) prior to injection. Accordingly, the present disclosure provides an article of manufacture, such as a kit, that provides the antigen component of the present immunogenic composition and an adjuvant in separate containers (e.g., pre-treated glass vials or ampules), and the adjuvant and the antigen component are mixed prior to injection: in some embodiments, a solution needed for resuspension of a lyophilized component, if any, is provided in the article. Alternatively, the antigen component and the adjuvant are mixed and provided in the same container, and the composition can be administered directly to subjects in need of vaccination. The article of manufacture may include instructions for use as well. In some cases, the contents of each container may be intended for separate administration as the first and second formulations.

In some embodiments, the recombinant S protein may be in dry form and the tocopherol-containing squalene emulsion adjuvant may be in liquid form. In such cases the contents of the first and second containers may be intended for combination to provide a co-formulation for administration. Alternatively, the recombinant S protein may be intended to be reconstituted prior to the contents of each container being used for separate administration as the first and second formulations.

The precise composition of liquid used for reconstitution will depend on both the contents of a container being reconstituted and the subsequent use of the reconstituted contents, e.g., if they are intended for administration directly or may be combined with other components prior to administration. A composition (such as those containing recombinant S protein or tocopherol-containing squalene emulsion adjuvant) intended for combination with other compositions prior to administration need not itself have a physiologically acceptable pH or a physiologically acceptable tonicity: a formulation intended for administration should have a physiologically acceptable pH and should have a physiologically acceptable osmolality.

The pH of a liquid preparation is adjusted in view of the components of the composition and necessary suitability for administration to the human subject. The pH of a formulation is generally at least 4, at least 5, or at least 5.5, such as at least 6. The pH of a formulation is generally 9 or less, 8.5 or less, or 8 or less, such as 7.5 or less. The pH of a formulation may be 4 to 9, 5 to 8.5, or 5.5 to 8, such as 6.5 to 7.4 (e.g., 6.5 to 7.1 such as about 6.8).

For parenteral administration, solutions should have a physiologically acceptable osmolality to avoid excessive cell distortion or lysis. A physiologically acceptable osmolality will generally mean that solutions will have an osmolality which is approximately isotonic or mildly hypertonic. Suitably the formulations for administration will have an osmolality of 250 to 750 mOsm/kg, 250 to 550 mOsm/kg, or 270 to 500 mOsm/kg, such as 270 to 400 mOsm/kg (e.g., about 280 mOsm/kg). Osmolality may be measured according to techniques known in the art, such as by the use of a commercially available osmometer, for example, the Advanced®: Model 2020 available from Advanced Instruments Inc. (USA).

Liquids used for reconstitution will be substantially aqueous, such as water for injection, phosphate-buffered saline and the like. As mentioned above, the requirement for buffer and/or tonicity modifying agents will depend on the on both the contents of the container being reconstituted and the subsequent use of the reconstituted contents. Buffers may be selected from acetate, citrate, histidine, maleate, phosphate, succinate, tartrate and TRIS. The buffer may be a phosphate buffer such as Na/Na₂PO₄, Na/K₂PO₄ or K/K₂PO₄. A suitable buffer is modified phosphate-buffered saline.

Suitably, the formulations used in the present invention have a dose volume of between 0.05 mL and 1 mL, such as between 0.1 and 0.6 mL, or have a dose volume of 0.45 to 0.55 mL, such as 0.5 mL. The volumes of the compositions used may depend on the subject, delivery route and location, with smaller doses being given by the intradermal route or if both the recombinant S protein and tocopherol-containing squalene emulsion adjuvant are delivered to the same location. A typical human dose for administration through routes such as intramuscular, is in the region of 200 μl to 750 mL, such as 400 to 600 μl, or is about 500 μl.

If two liquids are intended to be combined, for example for co-formulation if the recombinant S protein is in liquid form and the tocopherol-containing squalene emulsion adjuvant is in liquid form, the volume of each liquid may be the same or different. Volumes for combination will typically be in the range of 10:1 to 1:10, such as 2:1 to 1:2. Suitably the volume of each liquid will be substantially the same, such as the same. For example, a 250 μl volume of recombinant S protein in liquid form may be combined with a 250 μl volume tocopherol-containing squalene emulsion adjuvant in liquid form to provide a co-formulation dose with a 500 μl volume, each of the recombinant S protein and tocopherol-containing squalene emulsion adjuvant being diluted 2-fold during the combination.

Tocopherol-containing squalene emulsion adjuvants may therefore be prepared as a concentrate with the expectation of dilution by a liquid recombinant S protein containing composition prior to administration. For example, tocopherol-containing squalene emulsion adjuvant may be prepared at double-strength with the expectation of dilution by an equal volume of recombinant S protein containing composition prior to administration.

The concentration of squalene at administration may be in the range 0.8 to 100 mg per mL (e.g., 1.2 to 48.4 mg per ml).

Recombinant S protein and tocopherol-containing squalene emulsion adjuvant, whether intended for co-formulation or separate formulation, may be provided in the form of various physical containers such as vials or pre-filled syringes.

In some embodiments, the recombinant S protein, tocopherol-containing squalene emulsion adjuvant or kit comprising recombinant S protein and tocopherol-containing squalene emulsion adjuvant is provided in the form of a single dose. In other embodiments, the recombinant S protein, tocopherol-containing squalene emulsion adjuvant or kit comprising recombinant S protein and tocopherol-containing squalene emulsion adjuvant is provided in multidose form such as containing 2, 5, or 10 doses. Multidose forms, such as those comprising 10 doses, may be provided in the form of a plurality of containers with single doses of one part (e.g., the recombinant S protein) and a single container with multiple doses of the second part (e.g., tocopherol-containing squalene emulsion adjuvant) or may be provided in the form of a single container with multiple doses of one part (recombinant S protein) and a single container with multiple doses of the second part (tocopherol-containing squalene emulsion adjuvant).

It is common where liquids are to be transferred between containers, such as from a vial to a syringe, to provide “an overage” which ensures that the full volume required can be conveniently transferred. The level of overage required will depend on the circumstances but excessive overage should be avoided to reduce wastage and insufficient overage may cause practical difficulties. Overages may be of the order of 20 to 100 μl per dose, such as 30 μl or 50 μl. For example, a typical 10 dose container of doubly concentrated tocopherol-containing squalene emulsion adjuvant (250 μl per dose) may contain around 2.85 to 3.25 mL of tocopherol-containing squalene emulsion adjuvant.

Stabilizers or preservatives may be present. They may be useful where multidose containers are provided as doses of the final formulation(s) may be administered to subjects over a period of time. Such stabilizers/preservatives include, without limitation, parabens, thimerosal, chlorobutanol, bezalkonium chloride, and chelators (e.g., EDTA).

Recombinant S protein and tocopherol-containing squalene emulsion adjuvant in liquid form may be provided in the form of a multi-chambered syringe. The use of multi-chambered syringes provides a convenient method for the separate sequential administration of the recombinant S protein and tocopherol-containing squalene emulsion adjuvant. Multi-chambered syringes may be configured to provide concurrent but separate delivery of the recombinant S protein and tocopherol-containing squalene emulsion adjuvant, they may be configured to provide sequential delivery (in either order), or they may be configured to facilitate mixing prior to combined administration. In other configurations of multichambered syringes, the recombinant S protein may be provided in dry form (e.g., freeze-dried) in one chamber and reconstituted by the tocopherol-containing squalene emulsion adjuvant contained in the other chamber before administration. Examples of multi-chambered syringes may be found in disclosures such as WO2016/172396, although a range of other configurations are possible.

In some embodiments, the unit dosage is 1-50 or 5-50 (e.g., 2.5, 5, 10, 15, 30, or 45) μg recombinant S protein provided in 0.25 mL or 0.5 mL per dose.

In some embodiments, the unit dosage (i.e., a single dose) corresponds to 5 or 10 μg recombinant S protein formulated in phosphate buffered saline (q.s. 0.25 mL) with a concentration of 0.2% Tween 20R without preservatives or antibiotics. The antigen unit dosage may be supplied in multi-dose vials. They may be mixed with a single dose of an AS03 adjuvant (e.g., AS03_{A}, AS03_{B}, or AS03_{C}) prior to use.

In some embodiments, one unit dosage contains the ingredients as shown in Table A below.

TABLE A
CoV2 preS dTM Formulation (Non-Adjuvanted)

| Name of drug ingredient/substance | Quantity per dose | |
|---|---|---|
| Recombinant preS dTM protein | 2-50 | μg |
| Sodium phosphate monobasic monohydrate | 0.097 | mg |
| Sodium phosphate dibasic anhydrous | 0.26 | mg |
| Sodium chloride | 2.2 | mg |
| Polysorbate 20 (Tween 20 ®) | 0.55 | mg |

| | |
|---|---|
| Water for injection | q.s. final 0.25 mL |

In some embodiments, for each human vaccination by IM injection, 2.5 μg preS dTM or a variant in 0.25 mL of a sterile, clear and colorless PBS solution (see. e.g., Table A. Table 8 or Table 12 below) is mixed volume to volume with 0.25 mL of AS03 (AS03_{A}: see. e.g., Table 9) prior to injection, to reach a final injection volume of 0.5 mL. In further embodiments, the variant is the Beta variant (e.g., SEQ ID NO:13 without the signal sequence). In other embodiments, the dose of the AS03 adjuvant for mixing with the antigen solution is one half (AS03_{B}), one quarter (AS03_{C}), or one eighth (AS03_{D}) of 0.25 mL of the emulsion shown in Table 9; in such embodiments, a phosphate-buffered saline may optionally be added to the AS03 emulsion to reach a final volume of the adjuvant dose of 0.25 mL (see. e.g., Table 9).

In some embodiments, for each human vaccination by IM injection, 5 μg preS dTM or a variant in 0.25 mL of a sterile, clear and colorless PBS solution (see. e.g., Table A, Table 8 or Table 12 below) is mixed volume to volume with 0.25 mL of AS03 (AS03_{A}) prior to injection, to reach a final injection volume of 0.5 mL. In further embodiments, the variant is the Beta variant (e.g., SEQ ID NO: 13 without the signal sequence). In other embodiments, the dose of the AS03 adjuvant for mixing with the antigen solution is one half (AS03_{B}), one quarter (AS03_{C}), or one eighth (AS03_{D}) of 0.25 mL of the emulsion shown in Table 9; in such embodiments, a phosphate-buffered saline may optionally be added to the AS03 emulsion to reach a final volume of the adjuvant dose of 0.25 mL (see. e.g., Table 9).

In some embodiments, for each human vaccination by IM injection, 10 μg preS dTM or a variant in 0.25 mL of a sterile, clear and colorless PBS solution (see. e.g., Table A. Table 8 or Table 12 below) is mixed volume to volume with 0.25 mL of AS03 (AS03_{A}) prior to injection, to reach a final injection volume of 0.5 mL. In further embodiments, the variant is the Beta variant (e.g., SEQ ID NO: 13 without the signal sequence). In other embodiments, the dose of the AS03 adjuvant for mixing with the antigen solution is one half (AS03_{B}), one quarter (AS03_{C}), or one eighth (AS03_{D}) of 0.25 mL of the emulsion shown in Table 9: in such embodiments, a phosphate-buffered saline may optionally be added to the AS03 emulsion to reach a final volume of the adjuvant dose of 0.25 mL (see. e.g., Table 9).

In some embodiments, for each human vaccination by IM injection, 15 μg preS dTM or a variant in 0.25 mL of a sterile, clear and colorless PBS solution (see. e.g., Table A. Table 8 or Table 12 below) is mixed volume to volume with 0.25 mL of AS03 (AS03_{A}) prior to injection, to reach a final injection volume of 0.5 mL. In further embodiments, the variant is the Beta variant (e.g., SEQ ID NO: 13 without the signal sequence). In other embodiments, the dose of the AS03 adjuvant for mixing with the antigen solution is one half (AS03_{B}), one quarter (AS03_{C}), or one eighth (AS03_{D}) of 0.25 mL of the emulsion shown in Table 9; in such embodiments, a phosphate-buffered saline may optionally be added to the AS03 emulsion to reach a final volume of the adjuvant dose of 0.25 mL (see. e.g., Table 9).

In some embodiments, for each human vaccination by IM injection, 45 μg preS dTM or a variant in 0.25 mL of a sterile, clear and colorless PBS solution (see. e.g., Table A, Table 8 or Table 12 below) is mixed volume to volume with 0.25 mL of AS03 (AS03_{A}) prior to injection, to reach a final injection volume of 0.5 mL. In further embodiments, the variant is the Beta variant (e.g., SEQ ID NO: 13 without the signal sequence). In other embodiments, the dose of the AS03 adjuvant for mixing with the antigen solution is one half (AS03_{B}), one quarter (AS03_{C}), or one eighth (AS03_{D}) of 0.25 mL of the emulsion shown in Table 9: in such embodiments, a phosphate-buffered saline may optionally be added to the AS03 emulsion to reach a final volume of the adjuvant dose of 0.25 mL (see. e.g., Table 9).

In some embodiments, for each human vaccination by IM injection, a total of 10 μg of two different recombinant S protein (e.g., preS dTM or a variant such as one derived from B.1.351 (e.g., SEQ ID NO:13 without the signal sequence): 5 μg each) in 0.25 mL of a sterile, clear and colorless PBS solution (see. e.g., Table A, Table 8 or Table 12 below) is mixed volume to volume with 0.25 mL of AS03 (AS03_{A}) prior to injection, to reach a final injection volume of 0.5 mL. In other embodiments, the dose of the AS03 adjuvant for mixing with the antigen solution is one half (AS03_{B}), one quarter (AS03_{C}), or one eighth (AS03_{D}) of 0.25 mL of the emulsion shown in Table 9; in such embodiments, a phosphate-buffered saline may optionally be added to the AS03 emulsion to reach a final volume of the adjuvant dose of 0.25 mL (see. e.g., Table 9).

In some embodiments, the immunogenic composition is monovalent and contains 10 μg per dose of a single recombinant S protein (e.g., preS dTM or a preS dTM variant). The composition for injection comprises an AS03 adjuvant (see. e.g., Table 9). In further embodiments, the variant is the Beta variant (e.g., SEQ ID NO:13 without the signal sequence).

In some embodiments, the immunogenic composition is bivalent and contains two different recombinant S proteins (e.g., preS dTM and a preS dTM variant) at 5 μg each per dose. The composition for injection comprises an AS03 adjuvant (see. e.g., Table 9). In further embodiments, the variant is the Beta variant (e.g., SEQ ID NO:13 without the signal sequence).

In some embodiments, the immunogenic composition is trivalent and contains three different recombinant S proteins (e.g., preS dTM and two preS dTM variants) at 3.3 μg each per dose. The composition for injection comprises an AS03 adjuvant (see. e.g., Table 9). In further embodiments, one of the variants is the Beta variant (e.g., SEQ ID NO:13 without the signal sequence).

In some embodiments, the immunogenic composition is monovalent and contains a recombinant S protein (e.g., preS dTM) at 2.5 μg per dose. The composition for injection comprises an AS03 adjuvant (see. e.g., Table 9).

In some embodiments, the vaccine product of the present disclosure may be stored at 2-8° C.

### V. Use of the Vaccines

The present invention is generally intended for mammalian subjects, for example, human subjects.

The subject may be of any age. In one embodiment the subject is a human infant (up to 12 months of age). In one embodiment the subject is a human child (less than 18 years of age). In one embodiment the subject is an adult human (aged 18-59). In one embodiment the subject is an older human (aged 60 or greater). Doses administered to younger children, such as less than 12 years of age, may be reduced relative to an equivalent adult dose, such as by: 50%. In some embodiments, a 2.5 μg dose of antigen will be administered. In some embodiments, a 5 μg dose of antigen will be administered. In some embodiments, a 10 μg dose of antigen will be administered. In some embodiments a 15 μg dose of antigen will be administered. In some embodiments a 45 μg dose of antigen will be administered.

Suitably the subject is not infected with SARS-COV-2. In certain embodiments, the subject has not previously been infected with SARS-COV-2. In other embodiments the subject has previously been infected with SARS-COV-2 (e.g., developed COVID-19).

Subjects suitable for vaccination by the vaccine compositions of the present disclosure include humans susceptible for SARS-COV-2 infections. The amount of vaccine to be administered to the subjects can be determined in accordance with standard techniques well known to those of ordinary skill in the art, including the type of adjuvant used, the route of administration, and the age and weight of the subject. In some embodiments, a 2.5 μg dose of antigen mixed with tocopherol-containing squalene emulsion adjuvant will be administered. In some embodiments, a 5 μg dose of antigen mixed with tocopherol-containing squalene emulsion adjuvant will be administered. In some embodiments, a 10 μg dose of antigen mixed with tocopherol-containing squalene emulsion adjuvant will be administered. In some embodiments, a 15 μg dose of antigen mixed with tocopherol-containing squalene emulsion adjuvant will be administered. In some embodiments, a 45 μg dose of antigen mixed with tocopherol-containing squalene emulsion adjuvant will be administered.

The compositions may be administered in a single dose or in a series of doses (e.g., one to three primary doses with subsequent “booster” dose(s)). In some embodiments a first and second dose will be about 14 days (or about 2 weeks) to about six months apart. For example, the interval between doses may be 14-35 days (e.g., about 21 or 28 days) or about 2-5 weeks (e.g., about 3 or 4 weeks) or about one month apart.

In some embodiments, a single dose is a mixture of about 0.25 mL of an antigen composition as shown in Table A, Table 8 or Table 12 (containing 5 or 10 μg recombinant S protein) and an AS03 adjuvant (e.g., AS03_{A}, AS03_{B}, or AS03_{C}). In further embodiments, a subject is given two such doses, each dose being 21 days or 3 weeks part. In other further embodiments, a subject is given two such doses, each dose being 28 days or 4 weeks or one month part.

The vaccine composition is provided to the subject in a prophylactically effective amount, which may be administered in a single dose or in a series of doses. A “prophylactically effective amount” refers to the amount required to induce an immune response sufficient to prevent or delay onset, and/or reduce in frequency and/or severity, of one or more symptoms of COVID-19. In some embodiments, the amount elicits an immune response that reduces partially or completely the severity of one or more symptoms and/or time over which one or more symptoms are experienced by the subject, reduces the likelihood of developing an established infection after challenge, slows progression of illness, optionally extending survival, produces neutralizing antibodies to SARS-COV-2 and a SARS-CoV-2 S protein specific T cell response.

In some embodiments, the present disclosure provides a vaccination regimen as shown in Table B below. The regimen prevents or ameliorates COVID-19, such as one or more of its symptoms, or prevents or reduces the risk of hospitalization or death associated with COVID-19. In one regimen, a COVID-19 naïve or unvaccinated subject is vaccinated with IM with an immunogenic composition prepared by mixing 0.25 mL of an aqueous antigen component and 0.25 mL of an AS03 adjuvant (e.g., AS03_{A}, AS03_{B}, or AS03_{C}, whose volume may be made up with PBS to 0.25 mL, if need be). The 0.25 mL aqueous antigen component may be monovalent (MV) and comprises 10 μg of D614 preS dTM or B.1.351 (Beta) preS dTM formulated in PBS as shown in Table A. Alternatively, the aqueous antigen component is bivalent (BV) and comprises 5 μg of D614 preS dTM and 5 μg of Beta preS dTM formulated in PBS as shown in Table A. In the regimen, the subject is administered with the immunogenic composition twice, three weeks or four weeks apart.

### VI. Use of the Vaccines as Boosters

The present vaccine composition may be used as a universal booster. The present vaccine compositions may be used as boosters for previously administered COVID-19 vaccines, as part of a prime-boost vaccination regimen, e.g., a heterologous or homologous prime-boost vaccination regimen. The prime doses in the regimen (i.e., primary vaccines) may be vaccines that are based on mRNAs, DNAs, viral vectors (e.g., adenoviral vectors, adeno-associated viral vectors, lentiviral vectors, vesicular stomatitis viral vectors, vaccinia viral vectors, or measles viral vectors), peptides or proteins, viral-like particles (VLP), capsid-like particles (CLP), live attenuated viruses, inactivated viruses (killed vaccines), and the like. In some embodiments, the primary vaccine contains the same antigen as the booster vaccine (i.e., a homologous prime-boost vaccination regimen). A prime-boost regimen may be advantageous in part due to re-utilization, especially for a viral vector prime and to a qualitatively and quantitatively different immune profile provided by the boost. Such regimens are expected to lead to an enhanced outcome in terms of breadth, potency, and durability of the anti-viral immunity in vaccinated subjects.

Vaccines comprising genetic materials (e.g., mRNAs, DNAs, or viral vectors) for expressing a SARS-COV-2 antigen (e.g., an S protein antigen) in the body are collectively called “genetic vaccines.” For examples, genetic vaccines include those comprising mRNA, with or without chemical modifications or nucleotide analogs. The mRNA may be encapsulated (e.g., in lipid nanoparticles (LNP)) or complexed with a carrier or adjuvant (e.g., protamine or saponin). The mRNA may be self-replicating or non-self-replicating. The present vaccine compositions are useful as boosters for genetic vaccines, because genetic vaccines could elicit in the vaccinated subjects an anti-drug immune response that destroys and therefore reduces the efficacy of subsequent doses of the same vaccines. In such instances, the genetic vaccines cannot be administered to the same subjects repeatedly (e.g., seasonally).

In some embodiments of the present prime-boost regimen, the prime doses may be genetic vaccines encoding a recombinant S protein, which may include an ectodomain of the SARS-COV-2 S protein. In some embodiments, the recombinant S protein is a trimer of a polypeptide comprising a sequence from a SARS-COV-2 ectodomain or receptor-binding domain (RBD) and a trimerization sequence (e.g., the native SARS-COV-2 S trimerization domain). In some embodiments, the encoded recombinant S protein may comprise a signal peptide sequence (e.g., a signal peptide from SARS-COV-2 such as the S protein) that facilitates the secretion of the recombinant S protein from the producing cells in the vaccinated subject.

In some embodiments, the genetic vaccine encodes an S protein or an antigenic portion thereof that has one or more mutations as compared to a reference (e.g., naturally occurring) S protein for specific design purposes. For example, the encoded S protein may contain (i) mutations at the furin cleavage site to prevent furin cleavage (e.g., the “GSAS” (SEQ ID NO:6) mutations), (ii) mutations that alter endoplasmic reticulum (ER) retention, (iii) mutations that abrogate putative glycosylation, (iv) mutations that introduce an alternative signal peptide, and/or (v) mutations that stabilize the prefusion conformation of the S polypeptide (e.g., the “PP” mutations).

In some embodiments, the S protein encoded by the genetic vaccine may include naturally occurring mutations such as the D614G mutation and the other mutations described herein. In certain embodiments, the genetic vaccine may encode a recombinant S protein derived from a SARS-Cov-2 variant such as one described above.

In some embodiments, the genetic vaccine is Moderna COVID-19 Vaccine (mRNA-1273), Pfizer-BioNTech COVID-19 Vaccine (BNT162b2), Janssen COVID-19 Vaccine (Ad26.CoV2.S), and Vaxzevria (formerly COVID-19 Vaccine AstraZeneca).

In some embodiments of the present prime-boost regimen, the prime doses are killed vaccines, such as Sinovac-CoronaVac and the Sinopharm BIBP vaccine.

The prime-boost regimen comprises vaccination with a primary vaccine (e.g., a genetic vaccine or a subunit vaccine) and then one or more booster doses with the present protein vaccine. In some embodiments, the primary vaccine entails one administration (e.g., intramuscular, subcutaneous, intradermal, or intranasal administration) of the vaccine, or two administrations of the vaccine separated by a period of time (e.g., about 2, 3, 4, 5, 6, 7, 8, 9, or 10 weeks, or longer).

In some embodiments, a booster dose with the present recombinant protein may be given at least two weeks (e.g., four weeks, one month, two months, three months, four months, five months, six months, seven months, eight months, nine months, ten months, eleven months, one year, one and a half years, two years, three year, four years, five years, or longer) after the primary vaccination. For example, once a genetic vaccine (e.g., an mRNA or adenoviral-based vaccine) or a subunit vaccine is administered, a booster dose with the present protein vaccine may be given to the subject annually or semi-annually. For convenience, the booster vaccine may be co-administered with a flu vaccine annually (e.g., as separate formulations or co-formulation).

In some embodiments, the booster is a monovalent or multivalent immunogenic composition described herein, used with or without an adjuvant. In some embodiments, the booster is a monovalent immunogenic composition (e.g., one containing a recombinant S protein derived from the Wuhan strain or the South African variant). In other embodiments, the booster is a bivalent immunogenic composition (e.g., one containing a recombinant S protein derived from the Wuhan strain and a recombinant S protein derived from the South African variant).

In certain embodiments, a booster dose may be a 0.5 mL immunogenic composition made from mixing, prior to injection, 5 μg preS dTM and/or 5 μg variant in 0.25 mL of a sterile, clear and colorless PBS solution (see. e.g., Table A, Table 8 or Table 12 below) mixed volume to volume with 0.25 mL of AS03 (AS03_{A}). In further embodiments, the variant is the Beta variant (e.g., SEQ ID NO:13 with the signal sequence). The dose of the AS03 adjuvant for mixing with the antigen solution may also be one half (AS03_{B}), one quarter (AS03_{C}), or one eighth (AS03_{D}) of 0.25 mL of the emulsion shown in Table 9 below. In such embodiments, a phosphate-buffered saline may optionally be added to the AS03 emulsion to reach a final volume of the adjuvant dose of 0.25 mL (see. e.g., Table 9).

In certain embodiments, a booster dose may be a 0.5 mL immunogenic composition made from mixing, prior to injection, 2.5 μg preS dTM and/or 2.5 μg variant in 0.25 mL of a sterile, clear and colorless PBS solution (see. e.g., Table A, Table 8 or Table 12 below) mixed volume to volume with 0.25 mL of AS03 (AS03_{A}). In further embodiments, the variant is the Beta variant (e.g., SEQ ID NO: 13 with the signal sequence). The dose of the AS03 adjuvant for mixing with the antigen solution may also be one half (AS03_{B}), one quarter (AS03_{C}), or one eighth (AS03_{D}) of 0.25 mL of the emulsion shown in Table 9; in such embodiments, a phosphate-buffered saline may optionally be added to the AS03 emulsion to reach a final volume of the adjuvant dose of 0.25 mL (see. e.g., Table 9).

In certain embodiments, the primary vaccination is carried out with a subunit vaccine comprising a recombinant S protein, and the booster vaccine contains a lower amount of a recombinant S protein than the vaccine used for the primary (non-booster) vaccination. For example, the primary vaccination entails two shots, with 10 μg recombinant S protein per shot, separately by an interval (e.g., an interval of 2, 3, 4, 5, 6, 7, 8, or more weeks: 14-35 days), whereas a booster shot may contain just 2.5 or 5 μg recombinant S protein.

In some embodiments, the primary vaccination entails two shots of a 0.5 mL immunogenic composition prepared by mixing, prior to injection, 10 μg of preS dTM or a variant (or 5 μg of preS dTM plus 5 μg of a variant, e.g., the Beta variant, for a bivalent vaccine) in 0.25 mL of a sterile, clear and colorless PBS solution (see. e.g., Table A, Table 8 or Table 12 below) volume to volume with 0.25 mL of AS03 (AS03_{A}), with an interval (e.g., an interval of 3, 4, 5, 6, 7, 8, or more weeks) between the two shots. The dose of the AS03 adjuvant for mixing with the antigen solution may also be one half (AS03_{B}), one quarter (AS03_{C}), or one eighth (AS03_{D}) of 0.25 mL of the emulsion shown in Table 9: in such embodiments, a phosphate-buffered saline may optionally be added to the AS03 emulsion to reach a final volume of the adjuvant dose of 0.25 mL (see, e.g., Table 9). The subject is then given a booster vaccine at a later time (e.g., at least 3, 6, 7, 9, or 12 months after the second shot of the primary vaccination), wherein the booster vaccine may be a 0.5 mL immunogenic composition made from mixing, prior to injection, 2.5 or 5 μg of preS dTM or a variant (e.g., the Beta variant) in 0.25 mL of a sterile, clear and colorless PBS solution (see. e.g., Table A, Table 8 or Table 12) mixed volume to volume with 0.25 mL of AS03 (AS03_{A}). The dose of the AS03 adjuvant for mixing with the antigen solution may also be one half (AS03_{B}), one quarter (AS03_{C}), or one eighth (AS03_{D}) of 0.25 mL of the emulsion shown in Table 9; in such embodiments, a phosphate-buffered saline may optionally be added to the AS03 emulsion to reach a final volume of the adjuvant dose of 0.25 mL (see. e.g., Table 9).

In some embodiments, the vaccination regimen of the present disclosure is selected from the regimens described in Table B below:

TABLE B
Exemplary Vaccination Regimens

| | | Vaccine | | |
|---|---|---|---|---|
| | | | Ag | |
| Regimen | Subjects | Antigen (Ag) | amount (μg) | Adjuvant |
| 1 | Naïve | MV[D614] | 10 | AS03 |
| 2 | Primed 4 to ≤10 | MV[D614] | 5 | AS03 |
| | months prior* | | | |
| 3 | Primed 4 to ≤10 | MV[D614] | 5 | AS03 |
| 4 | months prior** | BV[D614 + | 2.5 + 2.5 | AS03 |
| | | Beta] | | |
| 5 | | MV[Beta] | 5 | AS03 |
| 6 | | MV[Beta] | 2.5 | AS03 |
| 7 | | MV[Beta] | 5 | None |
| 8 | | MV[Beta] | 5 | ½AS03 |
| 9 | | MV[Beta] | 2.5 | ½AS03 |
| 10 | Naïve | MV[D614] | 10 | AS03 |
| 11 | | BV[D614 + | 5 + 5 | AS03 |
| | | Beta] | | |
| 12 | | MV[Beta] | 10 | AS03 |

*Subjects who have been vaccinated (primed) with an mRNA-based vaccine or an adenoviral-based vaccine.
**Subjects who have been vaccinated (primed) with an mRNA-based vaccine, an adenoviral-based vaccine, or a recombinant S protein (e.g., preS dTM) vaccine.
MV: monovalent.
BV: bivalent.
D614: recombinant S protein derived from the Wuhan strain (e.g., preS dTM; SEQ ID NO: 10 without the signal sequence).
Beta: recombinant S protein derived from the Beta variant (e.g., SEQ ID NO: 13 without the signal sequence).
AS03: the dosage used here is 0.25 mL (i.e., AS03_{A}; see Table 9, infra).
½AS03 (also called AS03_{B}): half a dose of AS03_{A} (see Table 12, infra); i.e., 0.125 mL AS03 (see Table 9, infra).

In Table B above, regimens 2-9 are exemplary prime-boost regimens of the present disclosure. The regimens prevent or ameliorate COVID-19, such as one or more of its symptoms, or prevent or reduce the risk of hospitalization or death.

In some embodiments, a subject who has recovered from COVID-19 or has been vaccinated with a COVID-19 vaccine is given a booster at, for example, three, four, five, six, seven, eight, nine, ten, eleven, twelve, or more months after such recovery or vaccination. In further embodiments, the time for booster is about four to about ten months after such recovery or vaccination. In certain embodiments, the time for booster is about eight months after such recovery or vaccination. This subject may be administered by IM an immunogenic composition prepared by mixing 0.25 mL of an aqueous antigen component and an AS03 adjuvant (e.g., AS03_{A} or AS03_{B} (whose volume may be made up by PBS to 0.25 mL if needed)). In one embodiment, the 0.25 mL aqueous antigen component may be monovalent (MV) and comprises 2.5 μg of D614 preS dTM or Beta preS dTM formulated in PBS as shown in Table A, where the AS03 adjuvant is AS03_{A} or AS03_{B} (whose volume may be made up by PBS to 0.25 mL if needed). In one embodiment, the 0.25 mL aqueous antigen component may be monovalent (MV) and comprises 5 μg of D614 preS dTM or Beta preS dTM formulated in PBS as shown in Table A, where the AS03 adjuvant is AS03_{A} or AS03_{B} (whose volume may be made up by PBS to 0.25 mL if needed). In one embodiment, the aqueous antigen component is bivalent (BV) and comprises 2.5 μg of D614 preS dTM and 2.5 μg of Beta preS dTM formulated in PBS as shown in Table A, where the AS03 adjuvant is AS03_{A} or AS03_{B} (whose volume may be made up by PBS to 0.25 mL if needed).

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having.” “comprises,” or “comprising.” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

In order that this invention may be better understood, the following examples are set forth. These examples are for purposes of illustration only and are not to be construed as limiting the scope of the invention in any manner.

### EXAMPLES

### Example 1: Cloning of SARS-COV-2 S Encoding Sequences into Baculovirus Transfer Plasmids

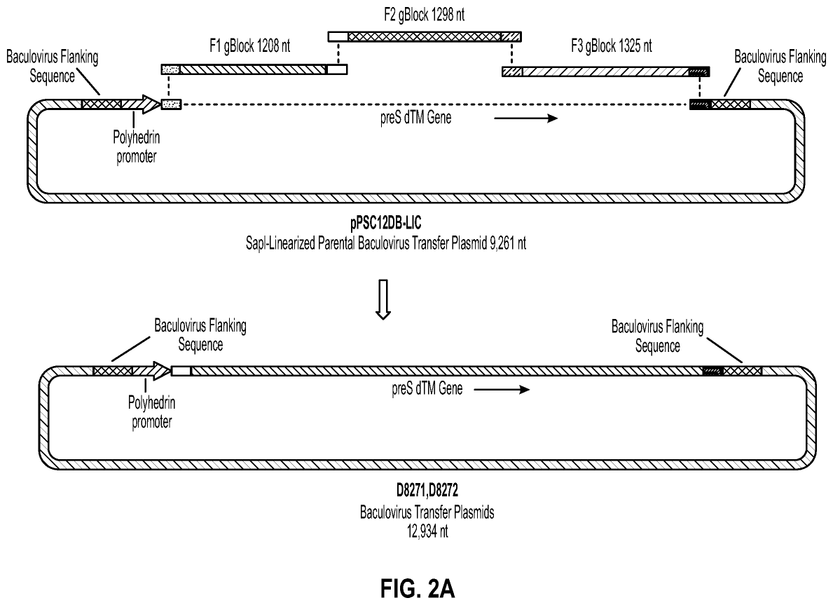

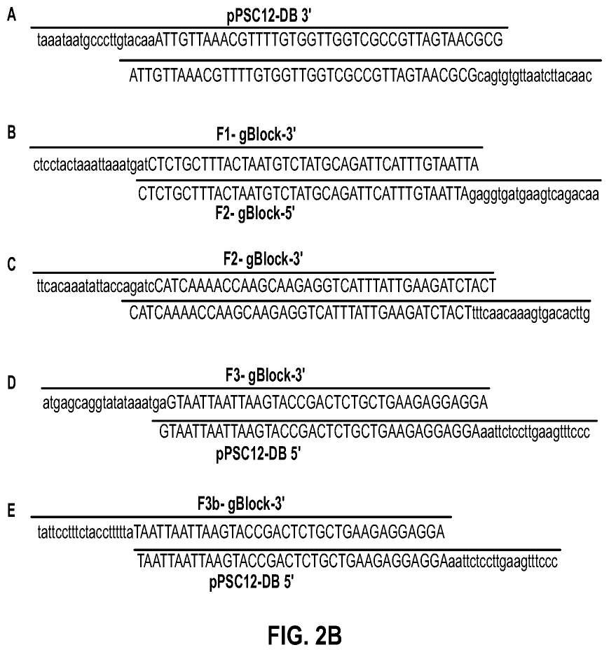

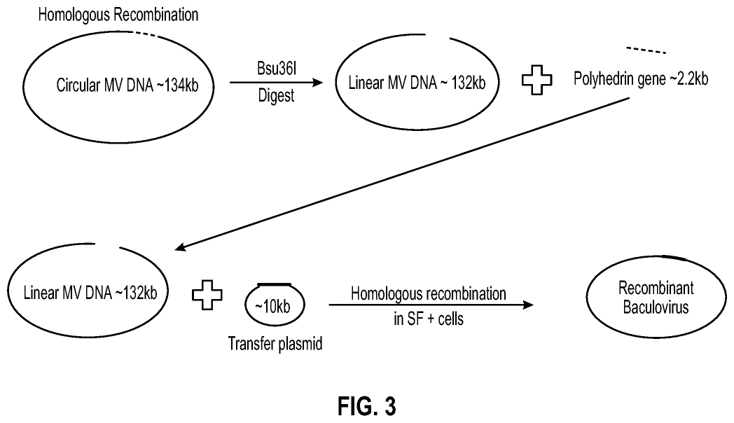

Gibson assembly (GA) was used to generate transfer plasmid harboring the indicated SARS-COV-2 spike glycoprotein modified from SARS-COV-2 spike glycoprotein, YP_009724390.1 from genome isolate Wuhan-Hu-1 GenBank NC045512. Three gene fragments (gBlocks) were designed for cloning into linearized SapI pPSC12 DB transfer vector, for each construct. The gBlock gene fragments have an overlapping 40 bp sequence at their junction sites and overlapping sequences with pPSC12 at 5′ and 3′ for gBlock fragment 1 and 3, respectively. gBlocks were synthesized by Integrated DNA Technologies (IDT). A depiction of the Gibson Assembly reaction is shown in (FIGS. 2A and 2B). Final transfer plasmid was confirmed via Sanger Sequencing by Eurofins Genomics. Site-directed mutagenesis may also be used to generate variant proteins.

### Example 2: Production and Purification of Recombinant S Protein

A recombinant baculovirus containing a sequence coding for preS dTM under the control of a polyhedrin promoter was used to infect S. frugiperda cells. Cells were grown at 27° C. to a density of 2.5×10⁶ cells/mL in PSFM medium (SAFC) and infected with 2% (volume/volume) of the recombinant baculovirus. Cells were harvested 72 hours post-infection by centrifugation for 15 minutes at 3,400×g. The supernatant was used for purification of recombinant S protein.

In one purification process, the supernatant containing the secreted recombinant SARS-COV-2 Spike protein was depth-filtered using a SUPRACAP 100 dual layer K250P/KS50P 5″ filter (Pall, #NP5LPDG41). The depth filtrate was concentrated 10× using 100 kDa Sartocon Slice Cassette, 0.1 m², flow rate of 200 mL/min at 15 psi followed by 5× diafiltration with 20 mM Tris: 50 mM NaCl, pH 7.4. The diafiltrate containing the SARS-CoV-2 Spike protein was purified by Capto™ Lentil Lectin (Cytiva) chromatography as the capture step purification. The Capto™ Lentil Lectin column was equilibrated with 20 mM Tris: 50 mM NaCl: 10 mM methyl-α-D-mannopyranoside, pH 7.4. Under these conditions, SARS-COV-2 Spike protein binds to Capto™ Lentil Lectin resin and contaminants flowed through the column. The column was washed with 20 mM Tris: 50 mM NaCl: 10 mM methyl-α-D-mannopyranoside, pH 7.4 to remove unbound proteins. The SARS-COV-2 Spike protein was eluted from the Capto™ Lentil Lectin column with elution buffer containing 20 mM Tris: 500 mM methyl-α-D-mannopyranoside, pH 7.4.

The Capto™ Lentil Lectin Eluate was further purified through Phenyl Sepharose™ HP Hydrophobic Interaction Chromatography resin (Cytiva) as the polishing step. The Capto™ Lentil Lectin eluate was adjusted to 750 mM ammonium sulfate concentration, 0.01% Triton X-100 concentration and loaded onto a Phenyl Sepharose HP column equilibrated with buffer containing 50 mM sodium phosphate: 750 mM ammonium sulfate: 0.01% v/v Triton X-100, pH 7.0. After loading, the Phenyl Sepharose HP column was washed with 50 mM sodium phosphate: 750 mM ammonium sulfate: 0.01% v/v Triton X-100, pH 7.0 to remove unbound contaminants. The SARS-COV2 Spike protein was eluted from the Phenyl Sepharose HP column elution buffer containing 50 mM sodium phosphate; 300 mM ammonium sulfate: 0.01% v/v Triton X-100, pH 7.0.

The Phenyl Sepharose HP Eluate was diluted 3.25× with distilled water and Q membrane filtration was performed using a single Mustang Q XT Acrodisc filter (Pall, #MSTGXT25Q16). Following the Q membrane filtration, TFF was performed using a Sartocon Slice 50 (Sartorius Stedim, #3_{D}91465050ELLPU). The Q Filtrate was concentrated to 0.25 mg/mL and then diafiltered 10× with 10 mM sodium phosphate buffer, pH 6.8-7.2. The TFF retentate containing the SARS-COV-2 Spike protein was formulated with 0.005% Tween 20 and sterile filtered using 0.2 μm filter and stored at 4° C. until use.

An alternative purification process uses CEX-HIC. Harvest may be accomplished with depth filtration (with or without an initial centrifugation step). Captured recombinant protein may then be further purified through ultrafiltration/diafiltration steps.

### Example 3: Emulsion Adjuvant Manufacture

Oil phase composed of squalene and D/L-alpha tocopherol was formulated under a nitrogen atmosphere. Aqueous phase, composed of modified phosphate-buffered saline and polysorbate 80, was prepared separately. Oil and aqueous phases were combined at a ratio of 1:9 (volume of oil phase to volume of aqueous phase) before homogenization and microfluidization (three passes through a microfluidizer at around 15,000 psi). The resulting emulsion was sterile filtered through two trains of two 0.5/0.2 μm filters in series (i.e., 0.5/0.2/0.5/0.2).

A final content of 42.76 mg/mL squalene, 47.44 mg/mL tocopherol and 19.44 mg/mL polysorbate 80 were targeted (see Table 9).

Particle size and polydispersity was determined by DLS to be within the range 140 to 180 nm and less than 0.2 respectively. Squalene and tocopherol contents were confirmed by HPLC and polysorbate 80 content by spectrophotometry to be within specification.

### Example 4: Pivotal Mouse Study

This example describes a study of a SARS-COV-2 recombinant protein vaccine formulation in mice. The vaccine formulation contained a SARS-COV-2 prefusion-stabilized S protein deleted for the transmembrane and cytoplasmic regions (preS dTM). The vaccine contained an AS03 adjuvant. This vaccine study investigated the dose response and adjuvant effect on both humoral and cell-mediated immunity. The study also compared the effect between a non-stabilized S ectodomain (deleted for transmembrane and cytoplamic region; “S dTM”) and preS dTM. S dTM contains SARS-COV-2 spike protein ECD S1 and S2 regions, with a His tag (Protein Sciences).

The mice used here were outbred female Swiss Webster mice, 6-8 weeks old. They were injected intramuscularly with 50 μL (25 μL of antigen solution plus 25 μL of AS03) of the vaccine formulation on Day 0 and Day 21.

The data below reflects targeted and actual antigen doses. After the experiment was run, a key polyclonal antibody reagent used to detect the SARS-COV-2 preS protein was found to also recognize glycosylated host cell proteins (HCP). As a result, the purity and HCP levels targeted were inaccurate and the concentration of SARS-COV-2 preS protein in the formulated vaccine product was significantly lower than planned. Table 1 shows the dosing regimen and Table 2 reflects the actual dose upon re-calculation as follows.

TABLE 1
Dosing Regimen for Mouse Study

| Group | | |
|---|---|---|
| (n = 10) | Targeted Antigen Dose (μg) | Adjuvant Dose (μg) |
| 1 | S dTM (4.5) | — |
| 2 | preS dTM (4.5) | AS03 (1,070) |
| 3 | preS dTM (1.5) | AS03 (1,070) |
| 4 | preS dTM (0.5) | AS03 (1,070) |
| 5 | preS dTM (0.167) | AS03 (1,070) |
| 6 | Diluent | — |

TABLE 2
CoV2-02_Ms Targeted and Actual
Doses of CoV2 preS dTM Antigen

| Injection | | CoV2 preS dTM | |
|---|---|---|---|
| day | Targeted doses (μg) | content %* | Actual doses (μg) |
| D 0 | 0.167/0.5/1.5/4.5 | 41 | 0.07/0.2/0.6/1.8 |
| D 21 | 0.167/0.5/1.5/4.5 | 26 | 0.04/0.13/0.4/1.17 |

*Actual doses were re-calculated based on new assays which differentiate structurally correct CoV2 preS dTM trimers from HCP impurities. The assays are based on ACE2 binding and/or HPSEC.

Because of the dosing adjustment based on new quantification assays, the actual doses were different for the D0 and D21 injections. For consistency, only the targeted doses are indicated in the text and figures.

Blood was drawn from the animals on Day −4. Day 21, and D 36. S-specific IgG. IgG1, and IgG2a levels were measured by ELISA, where the plates were coated with spike ECD containing the S1 and S2 regions (S dTM: Sino Biological). Titers are reported as the inverse of the last dilution eliciting OD values greater than 0.2. OD-0.2 value represents at least two times higher than the assay background. The ability of the serum antibodies to neutralize live virus was assessed first in a plaque reduction neutralization test (PRNT) at BSL3 using SARS-CoV-2 USA/WA1/2020 strain of the virus. Briefly, serum samples were heat inactivated at 56ºC for 30 minutes and diluted in diluent (DMEM/2% FBS). Diluted serum samples were mixed with an equal volume of SARS-COV-2 diluted to contain 30 PFU per well and incubated for 1 hour at 37ºC. Plates of confluent Vero E6 cells were inoculated with the serum-virus mixtures and incubated at 37ºC for 1 h. After incubation, plates were overlaid with 1 mL of the 0.5% methylcellulose media and incubated at 37ºC/5% CO₂ for 3 days. Then the plates were washed, fixed with ice cold methanol and stained with 0.2% crystal violet. Plates then were washed, dried and the neutralizing antibody titer was determined as the highest serum dilution that reduced the number of virus plaques in the test by 50% or greater.

The functional antibody responses elicited by the preS dTM vaccine were assessed using a pseudovirus neutralization assay. Serum samples were diluted and heat inactivated at 56° C. for 30 minutes. Diluted serum samples were mixed with a volume of reporter virus particle (RVP)-GFP (Integral Molecular) diluted to contain 300 infectious particles per well and incubated for 1 hour at 37ºC. 96-well plates of 50% confluent 293T-hsACE2 clonal cells were inoculated with the serum+virus mixtures and incubated at 37° C. for 72 h. Plates were scanned on a high-content imager and individual GFP expressing cells counted. The neutralizing antibody titer was reported as the reciprocal of the dilution that reduced the number of virus plaques in the test by 50%.

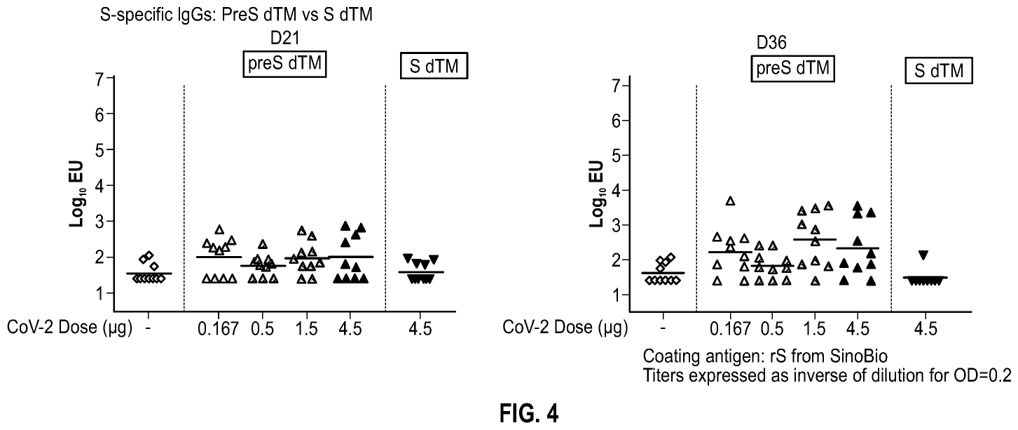

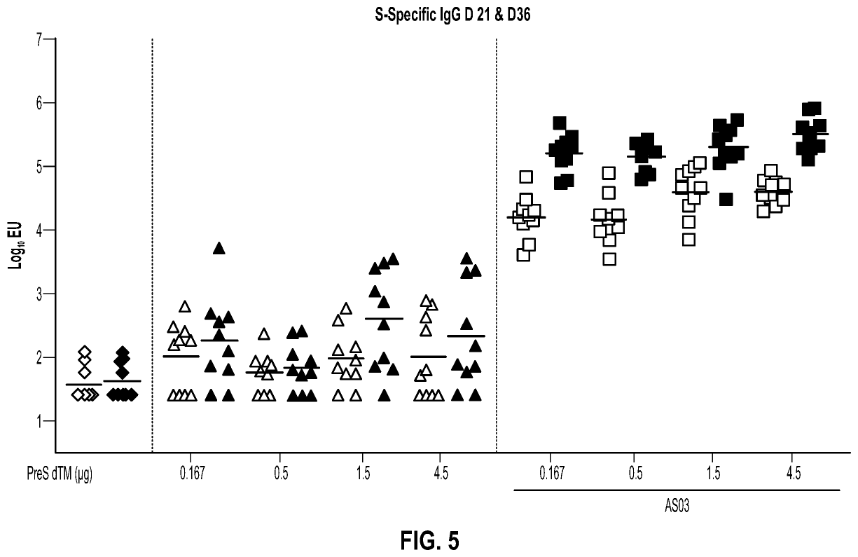

Without adjuvant, preS dTM and S dTM were not immunogenic, as demonstrated by very low or absent IgG and neutralizing antibody responses after 1 or 2 doses. The serum S-specific IgG levels were similar between the two antigens and there was no statistically significant titer change from Day 21 to Day 36 (FIG. 4). By contrast, a tocopherol-containing squalene emulsion adjuvanted preS dTM vaccine elicited high IgG responses after 1 dose (D21), across all doses tested (mean ranged from 4.1 to 4.6 Log₁₀ ELISA Unit (EU) in the different vaccine dose group). The responses were further increased by the second injection (D36), and IgG mean reached 5.1 to 5.5 Log₁₀ EU depending on the vaccine dose. Both the adjuvant effect (fold increase and P-value) and a booster effect were demonstrated. A modest dose-effect was observed for the IgG responses after 1 dose (×1.5, p-value<0.05) and after 2 doses. Thus, a tocopherol-containing squalene emulsion adjuvant significantly increased S-specific IgG titers in animals on both Day 21 and Day 36 induced by immunization with preS dTM, and Day 36 saw higher titers than Day 21 (FIG. 5). Titers obtained with a tocopherol-containing squalene emulsion adjuvant were significantly higher than those obtained without adjuvant. In summary, the dose-responsive effect of a tocopherol-containing squalene emulsion adjuvant-containing vaccine formulation was statistically significant, with p<0.05. However, the dose-response effect of the non-adjuvanted formulation was not statistically significant (p=0.7866). In short, whatever dose was used, a significant a tocopherol-containing squalene emulsion adjuvant effect was shown, with all dosages having p-values of <0.001.

Consistent with the IgG responses, the α-tocopherol-containing squalene emulsion-adjuvanted vaccine elicited robust neutralizing antibody responses after 2 doses. PRNT₅₀ titers were detected in all mice except two mice in the lowest dose groups (0.167 and 0.5 μg). Neutralizing mean range from 2.5 Log₁₀ in the lowest vaccine dose group (0.167 μg) to 3.5 Log₁₀ in the highest vaccine dose group (4.5 μg).

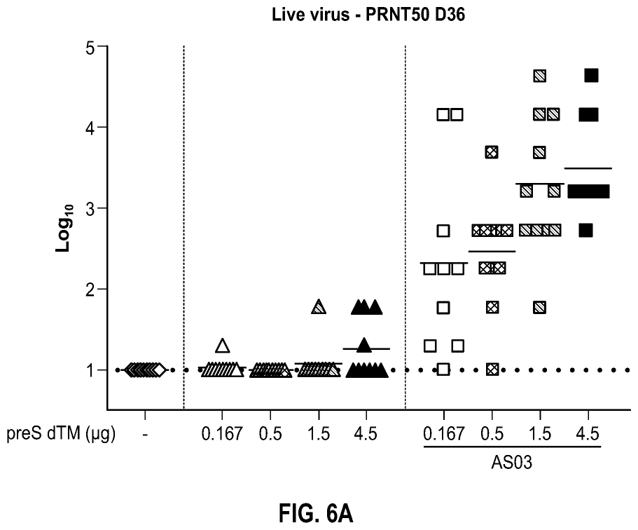

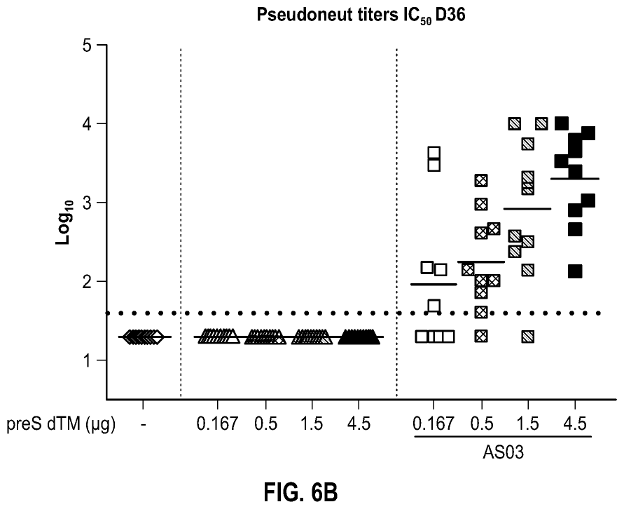

Consistent with the PRNT50 assay, pseudovirus neutralizing titers were detected in all the α tocopherol-containing squalene emulsion AS03-adjuvanted preS dTM immunized mice except one in the 0.5 μg group. Neutralization mean titers range from 2.6 Log₁₀ to 3.6 Log₁₀ in the highest vaccine dose group (4.5 μg). Thus, animals immunized with the adjuvanted formulation generated a significantly higher amount of SARS-COV-2 neutralizing antibodies by Day 36 in a dose-dependent manner FIGS. 6A and 6B.

### Example 5: Adjunct Mouse Study

This example describes a second study of a SARS-COV-2 recombinant protein vaccine formulation in mice. This study focused on evaluating cell-mediated immunity (CMI) in immunized mice. The mice used here were inbred female BALB/c mice, 6-8 weeks old. They were injected intramuscularly with 50 μL of the vaccine formulation on Day 0 and Day 14. The dosing regimens are shown as follows, with five mice per group. The preS dTM injected was targeted at 4.5 μg, with or without a tocopherol-containing squalene emulsion adjuvant. For consistency, only the targeted doses are indicated in the text and figures.

TABLE 3
CoV2-03_Ms Targeted and Actual
Doses of CoV2 preS dTM Antigen

| Injection | | CoV2 preS dTM | |
|---|---|---|---|
| day | Targeted doses (μg) | content %* | Actual doses (μg) |
| D 0 | 4.5 | 41 | 1.8 |
| D 21 | 4.5 | 26 | 1.17 |

*Actual doses were re-calculated based on new assays which differentiate structurally correct CoV2 preS dTM trimers from HCP impurities. The assays are based on ACE2 binding and/or HPSEC.

Blood was drawn from the animals on Day 0, Day 14, and Day 24. Spleens were harvested on Day 24 for CMI analysis, and spleen cells were stimulated with S1+S2 15-mer peptide pools (JPT) having 11 amino acid overlap. Cells were phenotyped by flow cytometry approach and cytokine production was assessed by intracellular cytokine staining (ICS). The biomarker panel evaluated is shown below.

TABLE 4
CMI Biomarker Panel

| Antibody | Format | Clone | Vendor |
|---|---|---|---|
| CD3 | BUV395 | 17A2 | BD |
| CD4 | PerCP-Cy5.5 | RM4-5 | Biolegend |
| CD8 | AF700 | 53-6.7 | BD |
| IFN-γ | FITC | XMG1.2 | BD |
| IL-5 | PE | TRFK5 | Biolegend |
| TNF | Pacific Blue | MP6-XT22 | Biolegend |
| IL-4 | APC | 11B11 | Biolegend |
| CD45RA | PE-Cy7 | RA3-6B2 | BD |
| CD14 | PE-Cy7 | Sa14-2 | Biolegend |
| IL-2 | BV605 | JES6-SH4 | BD |
| LIVE/DEAD | Near-IR (APC-Cy7) | | ThermoFisher |

To perform intracellular staining (ICS) the spleens were homogenized, red blood cells were lysed and the cells were rested for 1 hour at 37ºC and 5% CO₂. The splenocytes were then counted and 2×10⁶ cells were incubated for 6 hours at 37° C. and 5% CO₂ with Golgi Plug (BD Biosciences) under four conditions: no peptide stimulation (media only control), positive control stimulation, and stimulation with two individual spike peptide pools (JPT product PM-WCPV-S-1). Cells from each individual animal were stimulated with a Cell Activation Cocktail with Brefeldin A (Biolegend) as a positive control. Following stimulation, cells were washed and resuspended in Mouse BD Fc Block™ (clone 2.4G2) for 10 minutes at 4° C. Cells were then spun down, Fc block was removed, and cells were surface stained and live/dead stained for 30 minutes at 4° C. with an antibody cocktail containing: CD4 (RM4-5) PerCP-Cy5.5 (Biolegend), CD8 (53-6.7) AF700 (BD Biosciences), CD45R/B220 (RA3-6_{B}2) PE/Cy7 (BD Biosciences), CD14 (Sal4-2) PE/Cy7 (Biolegend) and LIVE/DEAD Fixable Near-IR Dead Cell Stain Kit (Invitrogen) in stain buffer (FBS) (BD Biosciences). After surface staining, cells were washed, fixed and permeabilized with Cytofix/Cytoperm solution (BD Biosciences) for 30 minutes at 4° C. Cells were then washed with 1× Perm/Wash solution (BD Biosciences), followed by intracellular staining for 30 minutes at 4° C., light protected, with a cocktail containing: CD3e (17_{A}2) BUV395 (BD Biosciences), IFN-γ (XMG1.2) FITC (BD Biosciences), TNF-α (MP6-XT22) Pacific Blue (Biolegend), IL-2 (JES6-5H4) BV605 (BD Biosciences), IL-4 (11_{B}11) APC (Biolegend), and IL-5 (TRFK5) PE (Biolegend) in 1× Perm/Wash buffer. Cells were then washed and resuspended in FACS buffer. Samples were run on an LSR Fortessa flow cytometer (BD Biosciences) and analysis was conducted on FlowJo software (version 10.6.1).

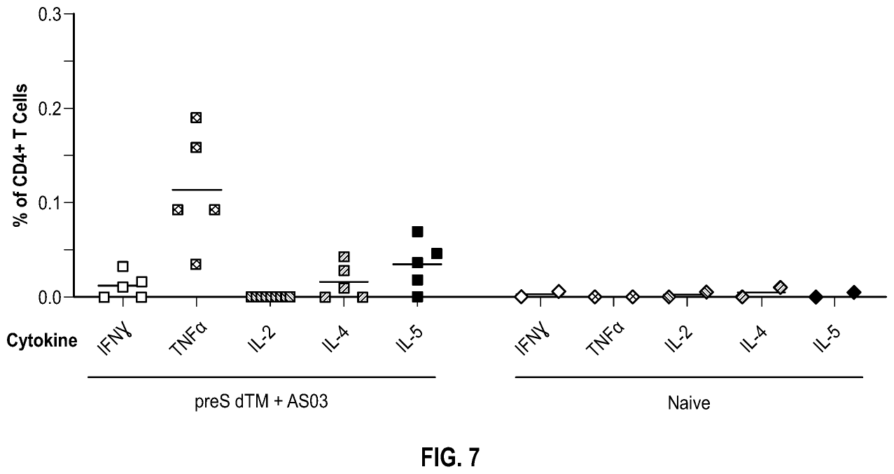

The ICS analysis indicated no or low S-specific CD4⁺ T cells after splenocyte stimulation with both S1 and S2 peptide pools in the AS03-adjuvanted vaccine immunized mice. Similar CD4⁺ T cell responses were observed with S1 and S2 peptide pools, below 0.05%, in the range of the non-specific signal detected in adjuvant-alone immunized mice; only S1 stimulation results are shown. No S-specific CD8⁺ T cell responses were detected. S-specific CD4⁺ T cells were detected in the α tocopherol-containing squalene emulsion adjuvanted vaccine immunized mice, with a predominance of TNF-α secreting cells (around 0.1%), and some IL-5-secreting cells (around 0.05%). No S-specific CD8⁺ T cell responses were detected, as expected for a recombinant antigen-based vaccine. The cytokine profile suggests a mixed Th1/Th2 response induced by the tocopherol-containing squalene emulsion-adjuvanted preS dTM vaccine. The cytokine profile suggests a mixed Th1/Th2 response induced by the AS03 adjuvanted preS dTM vaccine in BALB/c mice (FIG. 7).

### Example 6: Non-Human Primate Study

This example describes a study in non-human primates (NHP) evaluating the humoral immunity and CMI. The animals used here were Rhesus macaques, 4-12 years old. The NHPs were injected with a targeted dose of 15 μg of preS dTM mixed with the α-tocopherol-containing squalene emulsion adjuvant intramuscularly on Day 0 and Day 21 in a volume of 0.5 mL. Serum was collected on D4, D21, D28, and D35. For consistency, only the targeted doses are indicated in the text and figures.

TABLE 5
CoV2-02_NHP Targeted and Actual
Doses of CoV2 preS dTM Antigen

| Injection | Targeted | CoV2 preS dTM | Actual |
|---|---|---|---|
| day | doses (μg) | content %* | doses (μg) |
| D0 | 15 | 26 | 3.9 |
| D21 | 15 | 14 | 2.1 |

*Actual doses were re-calculated based on ACE2 binding quantification.

S-specific IgG levels were measured by ELISA, where the plates were coated with GCN4 pre-fusion form of spike protein (GeneArt).

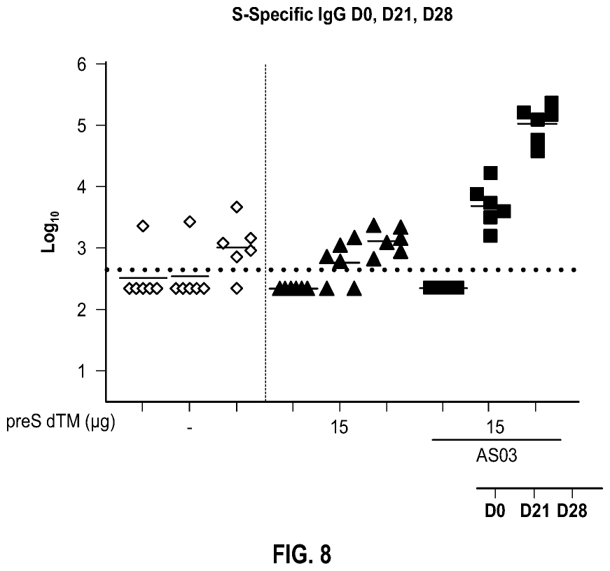

Consistent with the antibody responses observed in mice with preS dTM, no or very low responses were detected in absence of the adjuvant (FIG. 8). However, when formulated in AS03 adjuvant, the 15 μg preS dTM vaccine elicited high levels of IgGs binding to the prefusion S protein in all immunized monkeys, as early as 2 weeks post-dose 1 (mean titers of 3.7 Log₁₀ EU). The second immunization potently increased the IgG titers on D28 (mean titers of 5.1 Log₁₀ EU)

The functional antibody responses elicited by the preS dTM vaccine were assessed using a pseudovirus neutralization assay. Serum samples were diluted and heat-inactivated at 56° C. for 30 minutes. Diluted serum samples were mixed with a volume of reporter virus particle (RVP)-GFP (Integral Molecular) diluted to contain 300 infectious particles per well and incubated for 1 hour at 37° C. 96-well plates of 50% confluent 293T-hsACE2 clonal cells were inoculated with the serum+virus mixtures and incubated at 37° C. for 72 h. Plates were scanned on a high-content imager and individual GFP expressing cells counted. The neutralizing antibody titer was reported as the reciprocal of the dilution that reduced the number of virus plaques in the test by 50%.

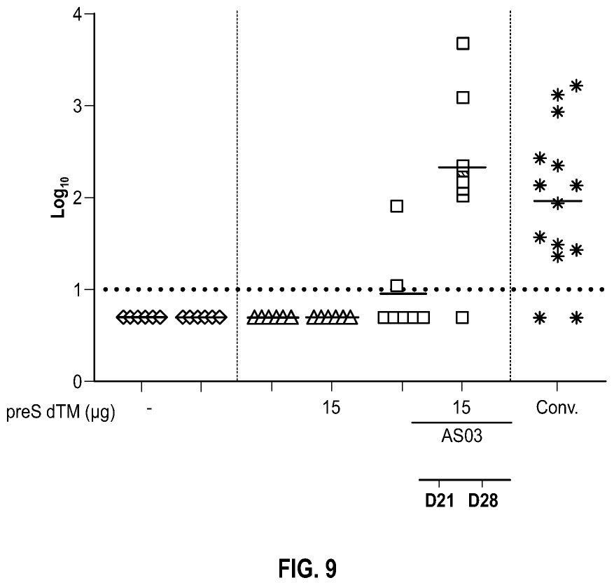

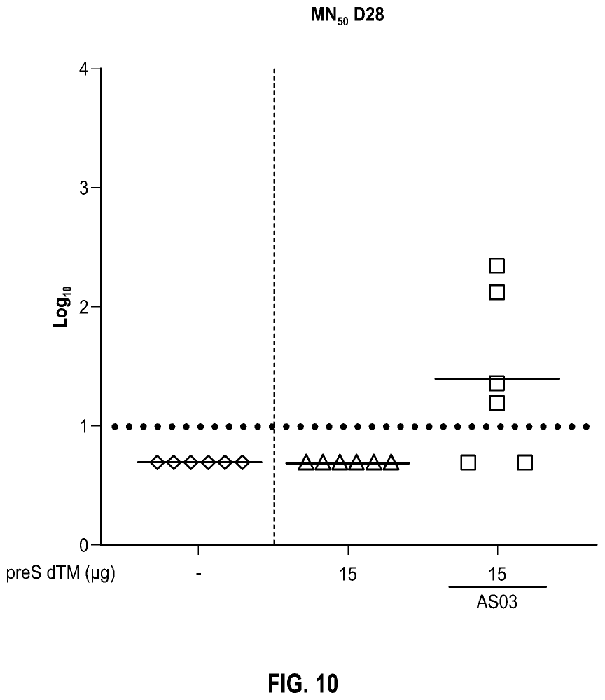

Three weeks post-dose 1, no pseudovirus neutralization titers were detected in any groups. However, following the second injection, pseudovirus neutralizing titers were detected in the α tocopherol-containing squalene emulsion adjuvant-adjuvanted preS dTM immunized macaques except one (mean titers of 2.1 Log₁₀ IC50). The neutralizing titers of the immunized rhesus were comparable to the titers observed for the panel of human convalescent (Conv.) sera (FIG. 9). Micro-neutralization (MN) assay data also show that AS03 significantly increased the titers of neutralization antibodies against wildtype SARS-CoV-2 virus (FIG. 10).

### Example 7.1: Hamster Study 1

Previous studies have indicated that hamsters consistently develop clinical signs after being challenged with SARS-COV-2, including lethargy, ruffled hair, rapid breathing, significant weight loss (up to 20% body weights), and diffuse alveolar damages from D2 to D14 in the lungs typical of SARS-COV-2 pneumonia in humans. Thus, hamsters are a suitable animal model for studying COVID vaccines. This example describes a study in hamster assessing the immunogenicity of a SARS-COV-2 recombinant protein vaccine formulation and the efficacy after a SARS-COV-2 viral challenge. The vaccine formulation contained preS dTM and AS03 adjuvant. This study investigated the specific antibody response and efficacy of one versus two doses of vaccine, as well as the adjuvant effect on humoral responses.

The animals used here were Golden Syrian Hamsters, 6-8 weeks old. They were injected intramuscularly with 75 μL (37.5 μL of antigen solution plus 37.5 μL of AS03) of the vaccine formulation in one-dose and two-dose cohorts, on Day 0 (for the two-dose cohort) and Day 21 (one- and two-dose cohorts). The dosages are shown as follows.

TABLE 6
Dosing Regimen for Hamster Study

| | | | |
|---|---|---|---|
| Group | Targeted | Adjuvant | Dosing |
| (n = 8) | Antigen Dose* (μg) | Dose (μg) | Schedule |

Two-Dose Cohort

| | | | |
|---|---|---|---|
| 1 | preS dTM (2.25) | AS03 (1.07) | D0/D21 |
| 2 | preS dTM (2.25) | — | D0/D21 |
| 3** | preS dTM (2.25) | AS03 (1.07) | D0/D21 |
| 4 | Diluent | — | D0/D21 |

One-Dose Cohort

| | | | |
|---|---|---|---|
| 5 | preS dTM (2.25) | AS03 (1.07) | D21 |
| 6 | preS dTM (2.25) | — | D21 |

*The effective dose was 0.6 μg for the one-dose cohort. The effective doses for the first and second doses of the two-dose cohort were 0.6 μg and 0.3 μg, respectively.
**n = 4. Sacrificed and PBMC collected on D23 for transcriptomics.

Blood was drawn from the animals before the first injection (baseline) and on, D35.

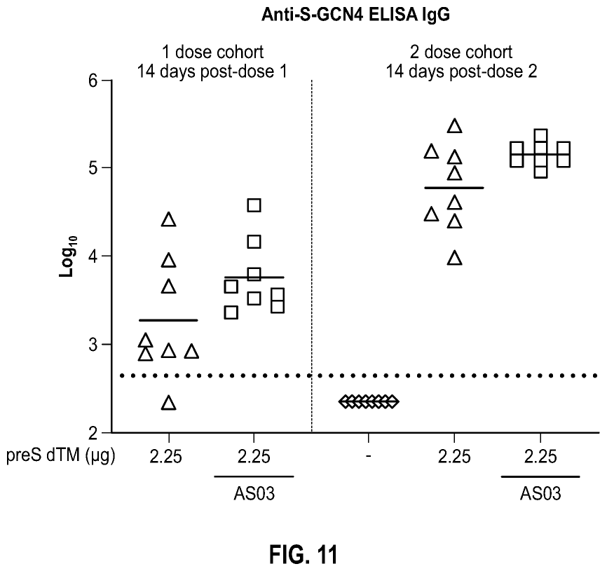

S-specific IgG levels were measured by ELISA, where the plates were coated with prefusion spike antigen (GCN4-stabilized) (GeneArt). Titers are reported as the inverse of the dilution giving an OD value equal to 0.2 (FIG. 11). OD=0.2 value represents the inverse of the dilution expressed in ELISA Units (EU).

All vaccinated hamsters developed S-specific IgG responses after one immunization except for 1 hamster immunized with non-adjuvanted preS dTM. AS03-adjuvanted preS dTM vaccine (2.25 μg targeted dose) induced higher S-specific IgG titers compared to the non-adjuvanted 2.25 μg targeted dose of preS dTM vaccine after one injection (mean of 3.8 Log₁₀ EU vs 3.3 Log₁₀ EU) or two injections (mean of 5.2 Log₁₀ EU vs 4.8 Log₁₀ EU). A significant difference was observed between the two-dose and one-dose vaccine regimen for the non-adjuvanted and AS03-adjuvanted vaccine with a 32- and 25-fold increase of S-specific IgG titers, respectively (p-value<0.001).

The functional antibody responses elicited by the preS dTM vaccine with and without AS03 were assessed using a pseudovirus neutralization assay (for both one- and two-dose cohorts). A pseudovirus neutralization assay was performed as follows: serum samples were diluted and heat-inactivated at 56° C. for 30 minutes. Further 2-fold serial dilutions of heat inactivated serum samples were mixed with a volume of reporter virus particle (RVP)-GFP (Integral Molecular) diluted to contain 300 infectious particles per well and then were incubated for 1 hour at 37° C. 96-well plates of 50% confluent 293T-hsACE2 clonal cells were inoculated with the serum+virus mixtures and incubated at 37ºC for 72 hours. Plates were scanned on a high-content imager and individual GFP expressing cells counted. The pseudovirus neutralizing antibody titer was reported as the reciprocal of the dilution that reduced the number of virus plaques in the test by 50% (ID₅₀, where ID₅₀=IC₅₀).

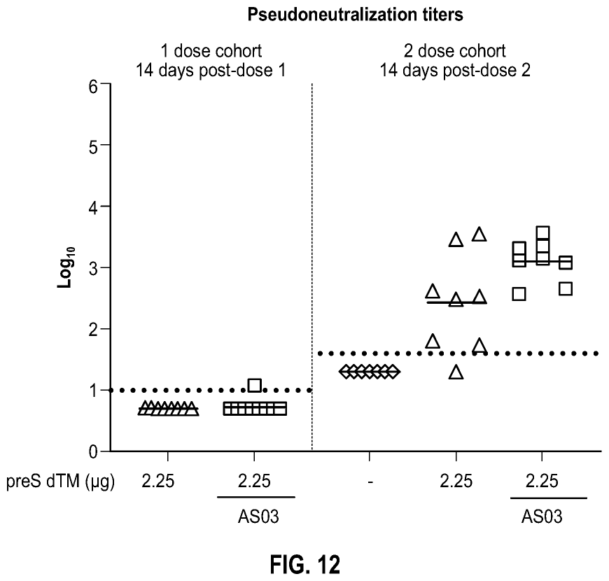

No pseudovirus neutralizing antibody responses were measured post-one injection (one-dose cohort) except for 1 hamster from AS03-adjuvanted preS dTM group, with a very low titer (FIG. 12). Non-adjuvanted preS dTM and AS03-adjuvanted vaccine elicited significant pseudovirus neutralizing antibody responses after 2 injections (two-dose cohort) with mean titers of 2.4 Log₁₀ and 3.1 Log₁₀, respectively. Pseudovirus neutralizing antibody titers were detected in all hamsters immunized with AS03-adjuvanted vaccine while titers were more heterogeneous and detected in 7/8 hamsters in the non-adjuvanted vaccine group. A modest but significant adjuvant effect of AS03 was observed (4.6-fold-increase: p=0.014).

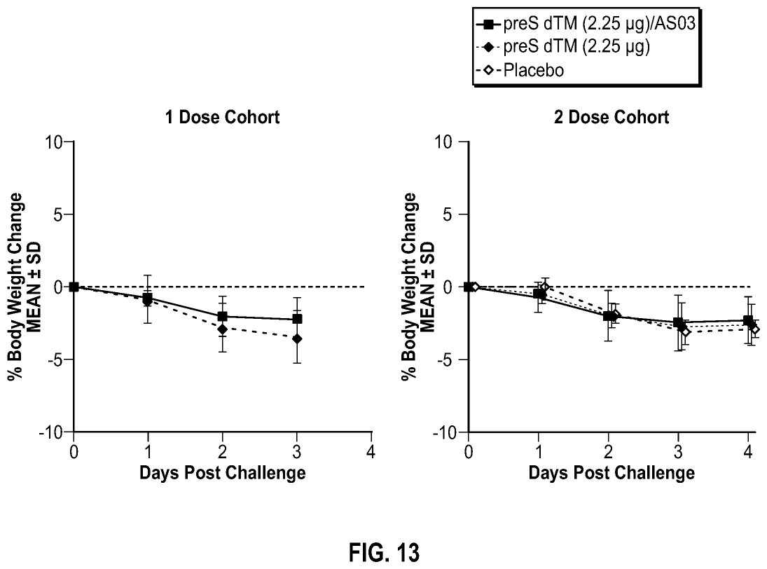

In order to assess vaccine efficacy, hamsters were challenged with 2.3×10⁴ PFU of SARS-COV-2 (USA-WA1/2020 strain) via a 100 μl intranasal administration. The clinical signs (body weight loss, general aspect, respiratory rate) were monitored daily post-challenge. At necropsy (either D4 post-challenge (n=4) or D7 post-challenge (n=7)), nares and lungs were collected for viral load assessment using qRT-PCR, and a lung pathology analysis was performed. Body weight loss was monitored up to 3 days post-challenge in one-dose cohort and up to 4 days post-challenge in two-dose cohort. The control group showed an unexpected very modest body weight loss, of about 3-4%, four days post-challenge (FIG. 13). The low body weight loss may be explained by the low pathogenic viral challenge stock used for the animal challenge. Because of the low delta in weight loss, no body weight loss differences could be observed post-challenge between control, non-adjuvanted or AS03-adjuvanted preS dTM vaccine groups, regardless number of immunizations.

Viral load content was assessed in nares and lungs only from two-dose cohort, 4- or 7-days post-challenge, using qRT-PCR measuring SARS COV-2 total RNA or subgenomic (sg) RNA. Of note, sgRNA is specific of active viral replication whereas total viral RNA accounts for both viral input and active replication.

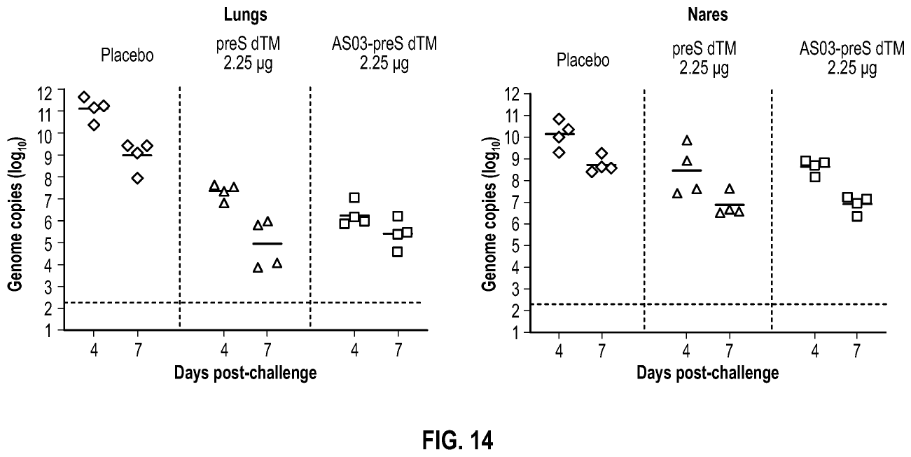

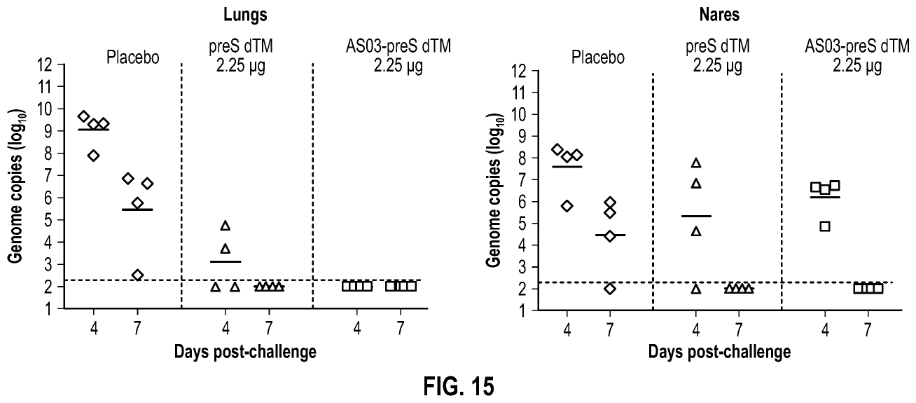

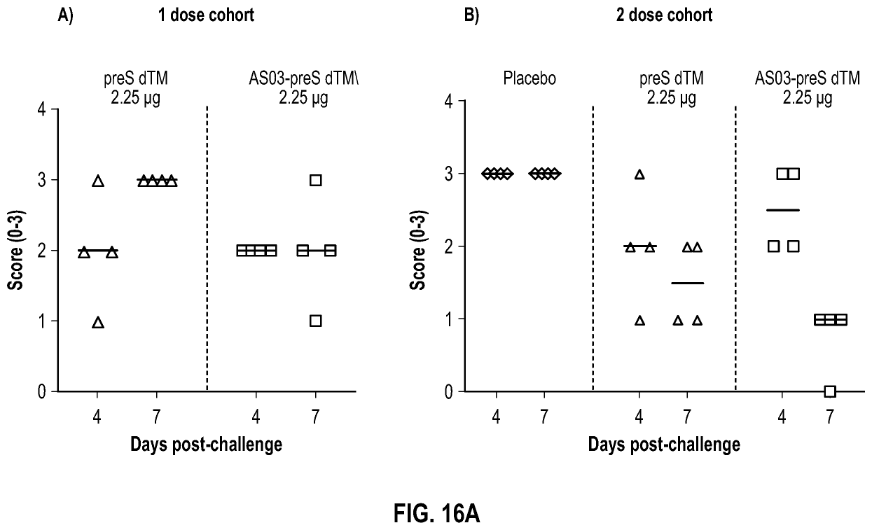

Despite the limited body weight loss, the control group displayed high sgRNA titers in lungs and nares on D4 (mean titers of 9.0 and 7.6 Log₁₀ copies/gram respectively), which were still detected on D7 in three out of four hamsters (mean titers of 5.4 and 4.5 Log₁₀ copies/gram, respectively) (FIG. 15). When compared to the control group, a strong reduction of viral load (FIG. 14). and viral replication (FIG. 15) in the lungs were observed in both vaccine groups (non-adjuvanted and AS03-adjuvanted) on D4 and D7 post-challenge. On D4, viral replication was only detected in two out of four animals in the non-adjuvanted vaccine group, and in none of the AS03-adjuvanted vaccine hamsters, indicating complete protection in the lungs with the AS03-adjuvanted vaccine. The reduction of viral replication in lungs in the vaccine groups was even more pronounced seven days post-challenge with no positive animals, while the control group still displayed a mean titer of 5.4 Log₁₀ sgRNA copies/gram. In nares, some reduction of viral load and viral replication were observed in vaccine groups on D4 and D7 post challenge compared to the control group. On D4, sgRNA mean titers were about 2 Log₁₀ lower in the vaccine groups, and on D7, all vaccinated animals were negative indicating the rapid viral clearance. These data suggested a clear protective effect of the immunization with the non-adjuvanted preS dTM and AS03-adjuvanted vaccines, against viral replication in lungs and nares, consistent with the neutralizing antibody responses measured in all vaccine groups.

Lung pathology was analyzed on D4 or D7 post-challenge for 4 hamsters/group from one- and two-dose cohorts. This analysis found that there was a clear decrease of the lung lesions on D7 for all vaccine formulations, which is even more pronounced for the 2.25 μg/AS03 dose, and there was a strong reduction of the viral protein expression in the pulmonary parenchyma. Table 7 shows the criteria used for the histopathology.

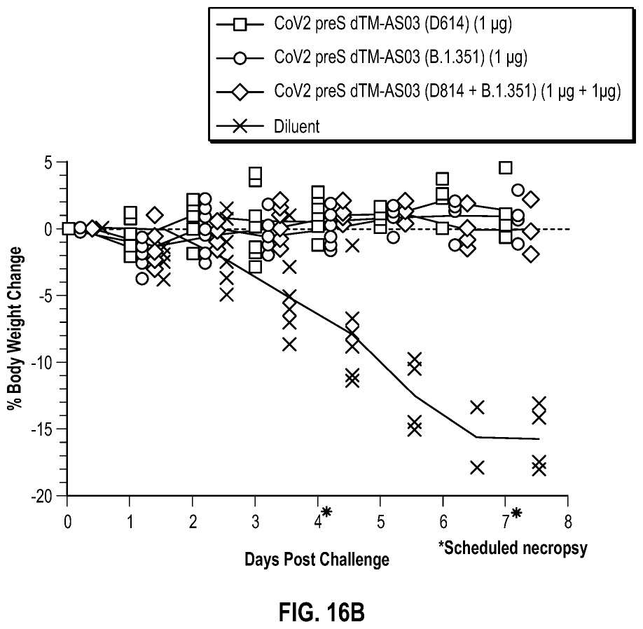

The control group displayed high pathology scores of 3 in the lung of all hamsters collected on D4 and D7 post-challenge, representing more than 50% of lung with severe lesions. (FIG. 16A). In the one-dose cohort, the non-adjuvanted preS dTM group immunized once displayed pathology scores varying from 1 to 3 on D4 post-challenge and were equal to 3 for all hamsters on D7 post-challenge. However, in the AS03-adjuvanted group immunized once, all pathology scores were reduced to 2 on D4 and were variable (between 1 and 3) on D7, indicating less lung pathology compared to the non-adjuvanted preS dTM group and to the control group. In the two-dose cohort, lower lung pathology was observed in the non-adjuvanted preS dTM and AS03-adjuvanted preS dTM groups compared to control group, especially on D7 (D4 scores ranging from 1 to 3 for both groups, and D7 scores from 1 to 2 in the non-adjuvanted group and from 0 to 1 in the AS03-adjuvanted group). The decrease of the pathology scores from D4 to D7 in the AS03-adjuvanted preS dTM group indicates a rapid resolution of the lung pathology.

TABLE 7
Histopathology Assessment

| Score | Histological findings in lungs |
|---|---|
| 0 | Normal lung |
| 1 | Mild: Findings described below affecting <25% of lung |
| 2 | Moderate: Findings described below affecting >25% |
| | of lung but <50% of lung |
| 3 | Severe: Findings described below affecting >50% of lung |

In multifocal to coalescing regions of lung, normal alveolar architecture is severely disrupted by marked numbers of inflammatory infiltrate composed of macrophages, lymphocytes, heterophils and syncytial cells admixed with high protein edema, hemorrhage, and cellular debris. Alveolar interstitial cells are often undergoing necrosis and loss with hyaline membrane formation and type 2 pneumocyte hyperplasia. Bronchiolar epithelial cells are alternately undergoing necrosis or severe hyperplasia with piling up. Necrotic cellular debris occupies the lumen of many bronchioles. Vascular walls are often disrupted and surrounded by inflammatory cells and edema. Multifocally, through out many sections, are collections of highly proliferative and pleomorphic epithelial cells.

### Example 7.2: Hamster Study 2

This example describes another study assessing the immunogenicity and efficacy of SARS-COV-2 recombinant protein vaccine formulations in hamsters. The vaccine formulations used in this study were either monovalent (containing the original D614 preS dTM (SEQ ID NO:10) or the B.1.351 preS dTM variant (SEQ ID NO:13)) or bivalent (containing the original D614 preS dTM and the B.1.351 preS dTM variant), both formulated with AS03 adjuvant. The vaccines' efficacy against two variants of concerns, Alpha (B.1.1.7) and Beta (B.1.351) was evaluated in hamsters at three weeks post-immunization.

In this study, groups of eight female Syrian Golden hamsters 6-8 weeks of age were immunized IM on DO and D21 with the three vaccine formulations at doses of 1 μg per recombinant protein component. Blood samples were collected before immunization, on D21, D35, and before challenge, to analyze the Spike-binding IgG and neutralizing antibody responses (Table 7.1). Four weeks after the second dose, all hamsters were inoculated IM with three SARS-COV-2 strains (D614G, B.1.351 (Beta), or B.1.1.7 (Alpha)) at doses previously determined to infect 100% of the animals and to induce between 10% and 20% of body weight loss during the first seven days post-challenge.

TABLE 7.1
Dosing Regimen for Second Hamster Study

| | | CoV2 preS | Dose per | |
|---|---|---|---|---|
| | Group | dTM | animal | |
| Cohort | (n = 8) | antigen | (μg) | Adjuvant |
| Part A | 1 | D614 | 1 | AS03 |
| B.1.351 | 2 | B.1.351 | 1 | AS03 |
| challenge | 3 | D614 + B.1.351 | 1 + 1 | AS03 |
| | 4 | Diluent only | N/A | N/A |
| Part B | 5 | D614 | 1 | AS03 |
| B.1.1.7 | 6 | B.1.351 | 1 | AS03 |
| challenge | 7 | D614 + B.1.351 | 1 + 1 | AS03 |
| | 8 | Diluent only | N/A | N/A |
| Part C | 9 | D614 | 1 | AS03 |
| D614 | 10 | B.1.351 | 1 | AS03 |
| challenge | 11 | D614 + B.1.351 | 1 + 1 | AS03 |
| | 12 | Diluent only | N/A | N/A |
| Control | 13 | N/A | N/A | N/A |

The clinical signs (body weight loss, general aspect, and respiratory rate) were monitored daily for seven days post-challenge. Four animals per group were sacrificed on D4 and the remaining four on D7 post-challenge to collect the lungs and nasal turbinate (or nares). Viral genomic RNA and subgenomic viral RNA were quantified by qRT-PCR. Histopathology in lungs were assessed on D4 and D7 post-challenge.

Body weights were measured in immunized and naïve hamsters daily after the challenge with the Beta (B.1.351) variant virus. For each hamster, the change in body weight compared to DO was calculated daily up to D7 post-challenge (time of final necropsy). The percent body weight changes are represented in FIG. 16B. The results showed a pronounced body weight loss in naïve hamsters, indicative of productive infection and pathology, while no immunized hamsters experienced any body weight loss after the challenge with the Beta variant. These data indicate that the three vaccine formulations tested, CoV2 preS dTM-AS03 (D614), (B.1.351) and (D614+B.1.351) conferred robust protection against infection and the associated pathology induced by the Beta variant in the hamster model.

### Example 8: Clinical Study

This example describes a Phase I/II clinical protocol for evaluating the safety and efficacy of a vaccine composition of the present disclosure. Participant, outcome assessors, investigators, laboratory personnel, and the majority of Sponsor study staff (except those involved in the ESDR and for concerned participants only) will be blinded to vaccine group assignment group (formulation and adjuvant; injection schedule will be unblinded. Those preparing/administering the study interventions will be unblinded to vaccine group assignment. Participants are randomized and stratified by age.

The composition comprises preS dTM (a trimer of a polypeptide of SEQ ID NO:10, without the signal peptide) with or without adjuvant. The vaccine composition is provided at two dosage strengths: Formulations 1 and 2, containing the preS dTM antigen at 5 μg (low dose) or 15 μg (high dose), respectively. The antigen composition is shown below:

TABLE 8
Antigen Compositions for Clinical Study

| | Quantity | |
|---|---|---|
| | (per 0.25 | |
| Component | mL Dose) | Function |
| Purified preS dTM | 5 or 15 μg | Active substance |
| recombinant protein | | |
| Sodium chloride | 4.4 mg | Inactive Ingredient: Buffer |
| Monobasic sodium phosphate | 0.195 mg | Inactive Ingredient: Buffer |
| Dibasic sodium phosphate | 1.3 mg | Inactive Ingredient: Buffer |
| Polysorbate 20 (Tween ® 20) | 27.5 μg | Stabilizer/Excipient |
| Baculovirus and Spodoptera | ≤19 μg | Residual |
| frugiperda cell proteins | | |
| Baculovirus and cellular DNA | ≤10 ng | Residual |
| Triton X-100 | ≤100 μg | Residual |

To evaluate the effect of adjuvant, AS03, an oil-in-water emulsion, is used. The unit dose strength for the adjuvant study groups is 5 μg and 15 μg of preS dTM. Each mono-dose vial of squalene-based a tocopherol-containing squalene emulsion adjuvant contains ingredients shown below. This emulsion has an oil phase containing squalene and D,L-α-tocopherol; and an aqueous phase containing a modified PBS and polysorbate 80. The amounts of ingredients shown below correspond to 250 μL of the AS03 bulk emulsion (i.e., AS03_{A}).

TABLE 9
AS03 Adjuvant Composition for Clinical Study

| | | |
|---|---|---|
| | | Quantity in |
| | | AS03 dose* |
| Ingredients | Concentration | (250 μL) |

Oil phase (10% v/v)

| | | |
|---|---|---|
| Squalene | 42.75 mg/mL | 10.69 mg |
| D,L-α-tocopherol | 47.45 mg/mL | 11.86 mg |
| — | — | Volume of |
| | | 0.025 mL |

Aqueous phase (90% v/v)

| | | |
|---|---|---|
| Polysorbate 80 | 19.44 mg/mL | 4.86 mg |
| Sodium chloride (NaCl) | 7.08 mg/mL (121 mM) | 1.77 mg |
| Potassium chloride (KCl) | 0.16 mg/mL (2.38 mM) | 0.04 mg |
| Disodium hydrogen | 1 mg/mL (7.14 mM) | 0.25 mg |
| phosphate (Na₂HPO₄) | | |
| Potassium dihydrogen | 0.16 mg/mL (1.30 mM) | 0.04 mg |
| phosphate (KH₂PO₄) | | |
| Water for injection | — | q.s. ad 0.225 mL |

| | |
|---|---|
| Average particle size | 140 to 180 nm |
| Manufacturing process | Microfluidization |

*This dose is also called AS03_{A}. AS03_{B}: one half of this dose (125 μL of the emulsion, with one half of the quantities of all ingredients). AS03_{C}: one quarter of this dose (62.5 μL of the emulsion, with one quarter of the quantities of all ingredients). AS03_{D}: one eighth of this dose (31.25 μL of the emulsion, with one eighth of the quantities of all ingredients).

The antigen composition and the adjuvant composition are mixed prior to use, with a total volume of 0.5 mL. Placebo is 0.5 mL per dose of 0.9% normal saline. The route of administration is intramuscular injection, at the deltoid muscle in the upper arm.

Each study intervention will be provided in an individual box (antigen and adjuvant or antigen and diluent (PBS) will be kitted together in a 2-vial box).

Participants are 18 years of age and older, healthy individuals and randomized within age groups. A small sentinel cohort made up of participants 18-49 years of age (Cohort 1) will receive a single dose. If safety data and laboratory measures to D09 in Cohort 1 are considered as acceptable based on unblinded data review, the remaining participants in

Cohort 1 and all participants in Cohort 2 will be enrolled. All participants will receive one injection of either one of the investigational study vaccine formulations or the placebo control at D01 (Vaccination [VAC] 1). Participants in Cohort 2 will receive a second injection of study vaccine formulation or placebo at D22 (VAC2). The duration of each participant's participation in the study will be approximately 365 days post-last injection.

COVID-19-like illness will be part of efficacy objective with active and passive surveillance. It is anticipated that the design of the candidate SARS-COV-2 antigen selected for this study will promote generation of robust neutralizing antibodies over binding antibodies. The inclusion of adjuvanted formulations is anticipated to further enhance the magnitude of neutralizing antibody responses and induce a balanced Th1/Th-2 T-helper cell responses. Taken together, these strategies mitigate by design theoretical risks of immune enhancement of viral infection. Individuals with chronic comorbid conditions considered to be associated with an increased risk of severe COVID-19 will be excluded.

A primary objective of the study is to evaluate immunogenicity of the vaccine composition by describing the levels and profiles of neutralizing antibodies at D01, D22, and D36. Neutralizing antibody titers will be measured with the neutralization assay. It is expected that the serum antibody neutralization titer post-vaccination at D22 and D36 will increase by about 2- to 4-fold relative to D01. Occurrence of neutralizing antibody seroconversion is defined as values below lower limit of quantification (LLOQ) at baseline with detectable neutralization titer above assay LLOQ at D22 and D36.

A secondary objective of the study is to evaluate immunogenicity of the vaccine composition by describing the binding antibody profile at D01, D22, D36, and D181 (Cohort 1) or D202 (Cohort 2), and D366 (Cohort 1) or D387 (Cohort 2) of each study intervention group, and describing the neutralizing antibody profile at D181 (Cohort 1) or D202 (Cohort 2) and D366 (Cohort 1) or D387 (Cohort 2) of each study intervention group. Binding antibody titers to full-length SARS-COV-2 spike protein will be measured for each study intervention group with the enzyme-linked immunosorbent assay (ELISA) method. It is expected that the fold-rise in anti-S antibody concentration [post/pre] will be 2 or more, or 4 or more at D22, D36, D181 (Cohort 1) or D202 (Cohort 2), and D366 (Cohort 1) or D387 (Cohort 2). Neutralizing antibody titers will be measured with the neutralization assay. It is expected that fold-rise in serum neutralization titer postvaccination at D181 (Cohort 1) or D202 (Cohort 2) and D366 (Cohort 1) or D387 (Cohort 2) relative to D01 will be 2 or more or 4 or more. Occurrence of neutralizing antibody seroconversion is defined as baseline values below LLOQ at baseline with detectable neutralization titer above assay lower limit of quantification at D181 (Cohort 1) or D202 (Cohort 2) and D366 (Cohort 1) or D387 (Cohort 2).

Another secondary object of the study is to evaluate efficacy by describing the occurrence of virologically-confirmed COVID-19-like illness and serologically confirmed SARS-COV-2 infection and evaluating the correlation/association between antibody responses to SARS-COV-2 Recombinant Protein and the risk of COVID-19-like illness and/or serologically confirmed SARS-COV-2 infection. Virologically confirmed COVID-19-like illness is defined by specified clinical symptoms and signs and confirmed by nucleic assay viral detection assay. Serologically-confirmed SARS-COV-2 infection is defined by SARS-COV-2-specific antibody detection in a non-S ELISA. Risk/protection correlation is based on antibody responses to SARS-COV-2 as evaluated using virus neutralization or ELISA, considering virologically confirmed COVID-19 like illness and/or serologically confirmed SARS-COV-2 infection as defined above.

An exploratory objective of the study is to evaluating immunogenicity by describing cellular immune response profile at D22 and D36 for each study intervention group in Cohort 2 and describing the ratio between neutralizing antibodies and binding antibodies. Th1 and Th2 cytokines will be measured in whole blood and/or cryopreserved PBMC following stimulation with full-length S protein and/or pools of S-antigen peptides. Ratio between binding antibody (ELISA) concentration and neutralizing antibody titer will be calculated.

### SARS-COV-2 Neutralizing Antibody Assessment

SARS-COV-2 neutralizing antibodies will be measured using a neutralization assay. In this assay, serum samples are mixed with constant concentration of the SARS-CoV2 virus. A reduction in virus infectivity (viral antigen production) due to neutralization by antibody present in serum samples can be detected by ELISA. After washing and fixation. SARS-COV-2 antigen production in cells can be detected by successive incubations with an anti-SARS-COV-2-specific antibody. HRP IgG conjugate, and a chromogenic substrate. The resulting optical density is measured using a microplate reader. The reduction in SARS-CoV-2 infectivity as compared to that in the virus control wells constitutes a positive neutralization reaction indicating the presence of neutralizing antibodies in the serum sample.

### SARS-COV-2 Spike Protein Antibody Serum IgG ELISA

SARS-COV-2 anti-S protein IgG antibodies will be measured using an ELISA. Microtiter plates will be coated with SARS-COV-2 pre-fusion form of spike protein antigen diluted in coating buffer to the optimal concentration. Plates may be blocked by the addition of a blocking buffer to all wells and incubation for a defined period. Following incubation, plates will be washed. All controls, reference, and samples will be pre-diluted with dilution buffer. The pre-diluted controls, reference and samples will then be further serially diluted in the wells of the coated test plate. The plates will be incubated for a defined period. Following incubation, plates will be washed, an optimized dilution of goat anti-human IgG enzyme conjugate will be added to all wells, and plates will be further incubated. Following this incubation, the plates will be washed, and enzyme substrate solution will be added to all wells. Plates will be incubated for a defined period to allow the substrate to develop. Substrate development will be stopped by the addition of a stop solution to each well. An ELISA microtiter plate reader will be used to read the test plates using assay specific SoftMax Pro templates. The average optical density (OD) value for the plate blank will be subtracted from all the ODs within each plate. The sample titers will be derived using the measured values of the blanks, controls, and the reference standard curve, which will be included on each assay plate within the run.

Cellular-Mediated Immunity (Using Whole Blood and/or PBMCs)

Cytokines will be measured in whole blood and/or cryopreserved PBMCs following stimulation with full-length S protein and/or pools of S-antigen peptides.

### COVID-19-Like Illness

COVID-19-like illness is defined as having (i) any one of the following (that persist for a period of at least 12 hours or reoccur within a 12-hour period): cough (dry or productive); fever: anosmia: ageusia: anosmia: Chilblains (COVID-toes): difficulty in breathing or shortness of breath: clinical or radiographic evidence of pneumonia; and any hospitalization with the clinical diagnosis of stroke, myocarditis, myocardial infarction, thromboembolic events (e.g., pulmonary embolism, deep vein thrombosis, and stroke), and/or purpura fulminans: or (ii) any two of the following (that persist for a period of at least 12 hours or reoccur within a 12-hour period): pharyngitis; chills; myalgia; headache; rhinorrhea; abdominal pain; and at least one of nausea, diarrhea, and vomiting.

### Virologically Confirmed COVID-19 Illness

Virologically confirmed COVID-19 illness is defined as a positive result for SARS-COV-2 by Nucleic Acid Amplification Test (NAAT) on a respiratory sample in association with a COVID-19-like illness.

### Serologically Confirmed SARS-COV-2 Infection

Serologically confirmed SARS-COV02 infection is defined as a positive result in serum for presence of antibodies specific to non-Spike protein of SARS-COV-2 detected by ELISA.

### SARS-COV-2 Nucleoprotein Antibody Serum IgG ELISA

SARS-COV-2 anti-nucleoprotein antibodies will be measured using an ELISA. Microtiter plates will be coated with SARS-COV-2 nucleoprotein antigen diluted in coating buffer to the optimal concentration. Plates may be blocked by the addition of a blocking buffer to all wells and incubation for a defined period. Following incubation, plates will be washed. All controls, reference, and samples will be pre-diluted with dilution buffer. The pre-diluted controls, reference and samples will then be further serially diluted in the wells of the coated test plate. The plates will be incubated for a defined period. Following incubation, plates will be washed, an optimized dilution of goat anti-human IgG enzyme conjugate will be added to all wells, and plates will be further incubated. Following this incubation, the plates will be washed, and enzyme substrate solution will be added to all wells. Plates will be incubated for a defined period to allow the substrate to develop. Substrate development will be stopped by the addition of a stop solution to each well. An ELISA microtiter plate reader will be used to read the test plates using assay specific SoftMax Pro templates. The average OD value for the plate blank will be subtracted from all the ODs within each plate. The sample titers will be derived using the measured values of the blanks, controls, and the reference standard curve, which will be included on each assay plate within the run.

### Nucleic Acid Amplification Test (NAAT) for COVID-19 Case Detection

In the assay, respiratory samples will be collected and the RNA is extracted. The purified template is then evaluated by an NAAT using SARS-COV-2 specific primers to specifically amplify SARS-COV-2 targets.

### Example 9: Immunogenicity and Safety of SARS-COV-2 Recombinant Protein Vaccine with AS03 Adjuvant in Adults 18 Years of Age and Older

This Example describes the protocol for a Phase II, randomized, modified double-blind, multi-center, dose-finding study conducted in adults 18 years of age and older to evaluate the safety, reactogenicity, and immunogenicity of 2 injections of preS dTM/AS03 adjuvanted vaccine (also referred to as “CoV2 preS dTM-AS03”) administered by intramuscular (IM) route. In this study (VAT00002), three different antigen doses (effective doses of 5 μg. 10 μg, and 15 μg of preS dTM) with a fixed dose of AS03 adjuvant (AS03_{A}) are evaluated. A two-injection schedule with doses administered 21 days apart is utilized in this study.

Reactogenicity is assessed in all participants by collecting solicited adverse events (AEs) for 7 days after each vaccination and unsolicited AEs through 21 days after the last vaccination. All participants will provide information on serious AEs, medically-attended AEs (MAAEs), and adverse events of Special Interest (AESIs) for the duration of the study. Neutralizing and binding antibodies are assessed in all participants over multiple time points over the duration of the study. Cellular and mucosal responses are assessed in a subset of participants. In addition, all episodes of COVID-19 are collected over the duration of the study.

Interim safety, reactogenicity, and immunogenicity data from this Phase II study will be used to decide on progression to Phase III and for selecting an antigen dose formulation to progress to Phase III. This interim analysis will occur after availability of reactogenicity data up to 21 days post-injection 2 and neutralizing antibody responses 14 days post-injection 2 in all participants.

Participants are categorized based on prior SARS-COV-2 infection as naïve (not previously infected) and non-naïve (evidence of previous infection) determined serologically (Roche Anti-N-Immunoassay and Roche Anti-S-Immunoassay) or virologically (Nucleic Acid Amplification Test [NAAT]). A naïve individual (no evidence of prior SARS-COV-2 infection) is defined as being negative by the Anti-N-immunoassay and the Anti-S immunoassay in serum sample(s) and a negative NAAT in a respiratory specimen at time of enrollment, while a non-naïve individual (evidence of prior SARS-COV-2 infection) is defined as being positive by the Anti-N-immunoassay OR the Anti-S-immunoassay in serum sample(s) or a positive NAAT in a respiratory specimen at time of enrollment.

### Objectives and Endpoints

### Primary Safety

To assess the safety profile of all participants in each age group and in each study intervention group, the following parameters are examined:
Presence of unsolicited systemic AEs reported in the 30 minutes after each vaccination;
Presence of solicited (pre-listed in the participant's diary card [DC] and [electronic] Case Report Form [CRF]) injection site reactions and systemic reactions occurring up to 7 days after each vaccination;
Presence of unsolicited AEs reported up to 21 days after the last vaccination;
Presence of serious adverse events (SAEs) throughout the study;
Presence of AESIs throughout the study; and
Presence of MAAEs throughout the study.

### Primary Immunogenicity

To assess the neutralizing antibody profile 14 days after the last vaccination (D36) in SARS-COV-2-naïve adults in each study intervention group, neutralizing antibody titers are measured in SARS-COV-2-naïve participants for each study intervention group against the D614G variant, including evaluating:
Individual serum neutralizing titer at D01 and D36;
Individual serum neutralization titer fold-rise post-vaccination relative to D01 at D36;
2-fold-rise and 4-fold-rise in serum neutralization titer [post/pre] (fold-rise≥2 and ≥4) at D36 relative to D01; and
Responders in SARS-COV-2 naïve, defined as participants who had baseline values below lower limit of quantification (LLOQ) with quantifiable neutralization titer above assay LLOQ at D36.

### Secondary Immunogenicity

Secondary objectives of the study include assessing (1) the neutralizing antibody profile at D22, D78, D134, D202, D292, and D387 in SARS-COV-2 naïve adults in each study intervention group: (2) the neutralizing antibody profile at D01, D22, D36, D78, D134, D202, D292, and D387 in each study intervention group for SARS-COV-2 non-naïve participants; and (3) the binding antibody profile at D01, D22, D36, D78, D134, D202, D292, and D387 in each study intervention group in SARS-COV-2 naïve and non-naïve participants.

The endpoints for secondary immunogenicity objectives (1) and (2) are neutralizing antibody titers in participants for each study intervention group against the D614G variant, including evaluating:
Individual serum neutralizing titer at each pre-defined time point;
Individual serum neutralization titer fold-rise post-vaccination relative to D01 at each pre-defined time point;
2-fold-rise and 4-fold-rise in serum neutralization titer [post/pre] (fold-rise≥2 and ≥4) at each pre-defined post-vaccination timepoint; and
Responders, defined as participants who had baseline values below LLOQ with quantifiable neutralization titer above assay LLOQ at each pre-defined post-vaccination timepoint and participants with baseline values above LLOQ with a 4-fold increase in neutralizing antibody titers at each pre-defined post-vaccination timepoint.

The endpoints for secondary immunogenicity objective (3) are binding antibody concentrations in participants for each study intervention group against the D614G variant, including evaluating:
Individual antibody concentration at each pre-defined time point;
Individual antibody fold-rise post-vaccination relative to D01 at each pre-defined post-vaccination time point;
2-fold-rise and 4-fold rise (fold-rise in antibody concentration [post/pre]≥2 and ≥4) at each pre-defined post-vaccination time point; and
Responders, defined as participants who had baseline values below LLOQ with quantifiable antibody concentration above assay LLOQ at predefined post-vaccination timepoints and participants with baseline values above LLOQ with a 4-fold increase in binding antibody concentration at each pre-defined post-vaccination timepoint.

### Secondary Safety

Secondary objectives of the study also include describing (1) the occurrences of laboratory-confirmed symptomatic COVID-19 in all participants in each study intervention group and (2) the occurrences of serologically-confirmed SARS-COV-2 infection in each study intervention group.

The endpoints for secondary safety objective (1) are:
Occurrences of laboratory-confirmed symptomatic COVID-19 (based on locally-confirmed or protocol-defined NAAT);
Occurrences of symptomatic COVID-19 episodes associated with hospitalization;
Occurrences of severe symptomatic COVID-19; and
Death associated with symptomatic COVID-19.

The endpoint for secondary safety objective (2) is occurrences of serologically-confirmed SARS-COV-2 infection.

### Exploratory Immunogenicity

The exploratory objectives of the study including (1) describing the ratio between neutralizing antibodies and binding antibodies: (2) assessing the T-cell cytokine profile at D01, D22 and D36 in a subset of participants: (3) further assessing the cellular immune response at D01, D22, D36, D134 and D387 in a subset of participants: (4) assessing the mucosal antibody response at D01, D22, D36, and D134 in a subset of participants; and (5) describing the neutralizing antibody response to emergent SARS-COV-2 variant strains.

The endpoint for exploratory immunogenicity objective (1) is the ratio between binding antibody (enzyme-linked immunosorbent assay [ELISA]) concentration and neutralizing antibody titer.

The endpoint for exploratory immunogenicity objective (2) is Th1 and Th2 cytokines measured in whole blood following stimulation with full-length S protein at D01, D22 and D36.

The endpoint for exploratory immunogenicity objective (3) is other cell-mediated immunity (CMI) assessments, which may be performed by Intracellular Cytokine Staining or/and enzyme-linked immunospot (ELISpot) assays.

The endpoints for exploratory immunogenicity objective (5) are neutralizing antibody responses to emergent variant strains, which will be measured in participants for each study intervention group, including evaluating:
Individual serum neutralizing titer at each pre-defined time point;
Individual serum neutralization titer fold-rise post-vaccination relative to D01 at each pre-defined time point;
2-fold-rise and 4-fold-rise in serum neutralization titer [post/pre] (fold-rise≥2 and ≥4) at each pre-defined post-vaccination timepoint; and
Responders, defined as participants who had baseline values below LLOQ with quantifiable neutralization titer above assay LLOQ at each pre-defined post-vaccination timepoint and participants with baseline values above LLOQ with a 4-fold increase in neutralizing antibody titers at each pre-defined post-vaccination timepoint.

### Overall Design

The overall design of the study is shown in Table 10.

TABLE 10
Overall Design

| | |
|---|---|
| Type of design | Parallel, multi-center |
| Phase | II |
| Study population | Adults 18 years of age and older |
| Level and | Modified double-blind (observer-blind) |
| method of | Blinding for vaccine group assignment: |
| blinding | participants, outcome assessors, Investigators, |
| | laboratory personnel, Sponsor trial staff, those |
| | administering the study intervention if not |
| | involvedin preparing study intervention. |
| | No blinding for vaccine group assignment: those |
| | preparing the study interventions. |
| Study | Participants are screened for eligibility criteria |
| intervention | at the time of inclusion. |
| assignment | The randomization is stratified by age groups |
| method | (18-59 years of age and 60 years of age and |
| | older), baseline SARS-COV-2 rapid |
| | serodiagnostic test positivity (Positive/Negative), |
| | and high-risk medical conditions (Yes/No). |
| | Eligible participants are randomly assigned to |
| | one of three study groups in a 1:1:1 ratio, |
| | corresponding to one of three formulations of |
| | CoV2 preS dTM-AS03 vaccine (with different |
| | antigen doses). |

A total of 720 participants are planned to be enrolled. After stratification by age-group (18-59 years and ≥60 years), baseline SARS-COV-2 rapid serodiagnostic test positivity (Positive/Negative [as determined at the time of enrollment]) and high-risk medical conditions (Yes/No), participants will be randomly assigned to the study groups.

For all study arms, half of the participants will be 18-59 years of age and half of the participants will be 60 years or older. Additionally, up to 20% of participants in each study group may be test-positive on the rapid serodiagnostic test at enrollment. A randomized subset of 120 rapid diagnostic test-negative participants stratified by age-group (20 participants/study group/age-group) will provide samples for cellular immune response and mucosal antibody assessments.

### Intervention Groups and Duration

The intervention groups and duration are summarized in Table 11 below. The amount of the preS dTM antigen used is indicated for each invention group.

TABLE 11
Planned Sample Size and Approximate Size of Subsets

| | | Participants | | |
|---|---|---|---|---|
| | | Study Intervention Groups | | |
| | | Group 1 | Group 2 | Group 3 |
| | | 5 μg antigen | 10 μg antigen | 15 μg antigen |
| Total Overall | | 240 | 240 | 240 |
| SARS-CoV-2 | Negative | At least 192* | At least 192* | At least 192* |
| serodiagnostic | Positive | Up to 48† | Up to 48† | Up to 48† |
| test at | | | | |
| baseline | | | | |
| Age Groups | adults (18-59 | 120 | 120 | 120 |
| | years) | | | |
| | older adults | 120 | 120 | 120 |
| | (≥60 years) | | | |

| | | | |
|---|---|---|---|
| Cellular Immunity and | 40 | 40 | 40 |
| Mucosal subset‡ | (20/age | (20/age | (20/age |
| | group) | group) | group) |

*96 per age group.
†up to 24 per age group.
‡Participants SARS-CoV-2 serodiagnostic test negative at baseline would be randomized to this subset.

All participants receive two vaccine injections given at 3 weeks apart: the first injection is at D01 (Vaccination [VAC] 1) and the second injection is at D22 (VAC2). Blood samples are collected from all participants prior to each injection, 14 days, 2 months, 4 months, 6 months, 9 months, and 12 months after last injection. Blood samples collected from all participants are used for serological assessments in the study. Whole blood, peripheral blood mononuclear cells (PBMCs), and saliva samples are collected from a subset of participants to assess cellular immune responses and mucosal antibody responses.

All participants are followed for the duration of the trial to capture occurrences of COVID-19 through passive surveillance wherein participants will be instructed to contact the site if they experience symptoms/conditions of a COVID-19-like illness at any time during the study. In addition, active surveillance is undertaken in all participants wherein all participants are contacted once every 2 weeks starting after the D43 contact to enquire about development of COVID-19-like illness.

The duration of each participant's participation in the study will be approximately 365 days post-injection 2 (i.e., approximately 386 days total).

### Analysis Sets

The following subgroup definitions of SARS-COV-2 Naïve and Non-Naïve at D01 or both D01 and D22 timepoints are applied for all randomized participants:

The participant analysis sets are:
1. SARS-COV-2 Naïve at baseline (Naïve-D01)
   Negative by the anti-S immunoassay (Roche Elecsys) on D01 serum sample,
   Negative by the anti-N immunoassay on D01 serum sample, and
   Negative NAAT for SARS-COV-2 on respiratory sample collected on D01.
2. SARS-COV-2 Non-Naïve at baseline (Non-Naïve-D01)
   Positive by the anti-S immunoassay (Roche Elecsys) on D01 serum sample,
   Positive by the anti-N immunoassay on D01 serum sample, or
   Positive NAAT for SARS-COV-2 on respiratory sample collected on D01
3. SARS-COV-2 Naïve at second injection (Naïve-D01+D22)
   Negative by the anti-S immunoassay (Roche Elecsys) on D01 serum sample,
   Negative by anti-N immunoassay on D01 and D22 serum sample, and
   Negative NAAT for SARS-COV-2 on respiratory sample collected on D01 and D22.
4. SARS-COV-2 Non-Naïve at second injection (Non-Naïve-D01/D22)
   Positive by the anti-S immunoassay (Roche Elecsys) on D01 serum sample,
   Positive by the anti-N immunoassay on D01 or D22 serum sample, or
   Positive NAAT for SARS-COV-2 on respiratory sample collected on D01 or D22.

The defined populations include the following:
1. Full analysis set (FAS): all randomized participants who receive at least one study injection; participants will be analyzed according to the intervention to which they were randomized.
2. Per-protocol analysis set (PPAS): subset of the FAS: participants presenting with at least one of the following criteria will be excluded from the PPAS:
   Participant did not meet all protocol-specified inclusion criteria or met at least one of the protocol-specified exclusion criteria,
   Participants did not receive both injections,
   Participant received a vaccine other than the one that he/she was randomized to receive,
   Preparation and/or administration of vaccine was not done as per protocol,
   Participant did not receive vaccine in the proper time window,
   Participants who did not collect D01 or D36 blood sample,
   Participant received the second injection despite meeting any of the definitive contraindication criteria, and
   Participant receives an authorized/approved COVID-19 vaccine prior to D36.

### Example 10: Phase II Data

Data from the Phase II clinical study, performed in accordance with the protocol described in the above Example, show that CoV2 preS dTM AS03 successfully demonstrates strong immune responses across all adult age groups with 720 volunteers.

In the study, CoV2 preS dTM was provided in an aqueous phosphate-buffered saline (PBS) solution. Each dose of the antigen (5, 10, or 15 μg) was provided in 0.25 mL of the solution and mixed with 0.25 mL of AS03 at bedside. Each vaccination dose, post mixture, had the composition shown in Table 12. The antigen solution and the adjuvant were provided in separate vials in a 2-vial box. The vials were stored at 2 to 8ºC.

TABLE 12
CoV2 preS dTM AS03 Formulation

| Role of drug | Name of drug | | |
|---|---|---|---|
| ingredient/ | ingredient/ | Quantity | Quantity |
| substance | substance | expression | per dose |
| Antigen | Recombinant preS | ≥(not | 5 μg (low dose) |
| | dTM protein | less | or |
| | (SARS-CoV-2 | than) | 10 μg (medium |
| | Wuhan D614 | | dose) or |
| | strain) | | 15 μg (high dose) |
| Excipient—Buffer | Monobasic sodium | Equal | 0.0975 mg |
| | phosphate | | |
| Excipient—Buffer | Dibasic sodium | Equal | 0.65 mg* |
| | phosphate | | |
| | dodecahydrate | | |
| Excipient—Buffer | Sodium chloride | Equal | 2.2 mg |
| Excipient—Buffer | Polysorbate 20 | Equal | 55 μg |
| Solvent/Diluent | Water for injection | Sufficient | 0.25 mL |
| | | quantity | |
| | | (g.s.) | |
| Adjuvant | AS03_{A} (10.69 mg | Sufficient | 0.25 mL |
| | squalene, 11.86 | quantity | |
| | mg DL-α- | | |
| | tocopherol, and | | |
| | 4.86 mg | | |
| | poly sorbate 80) | | |

*This corresponds to 0.26 mg dibasic sodium phosphate anhydrous, which can also be used to prepare the formulation.

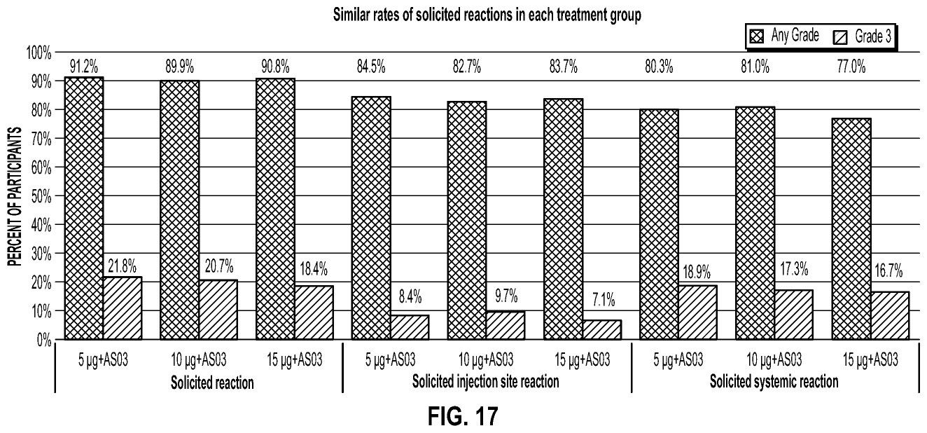

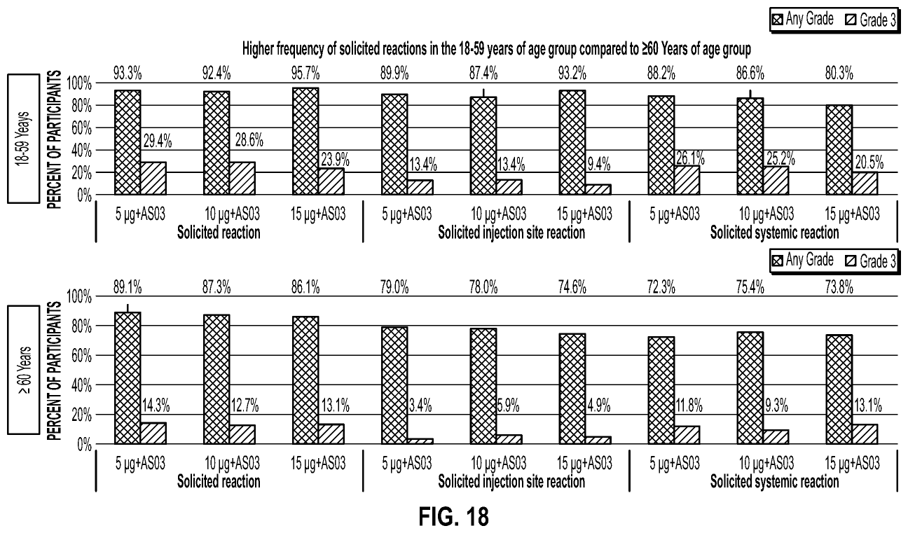

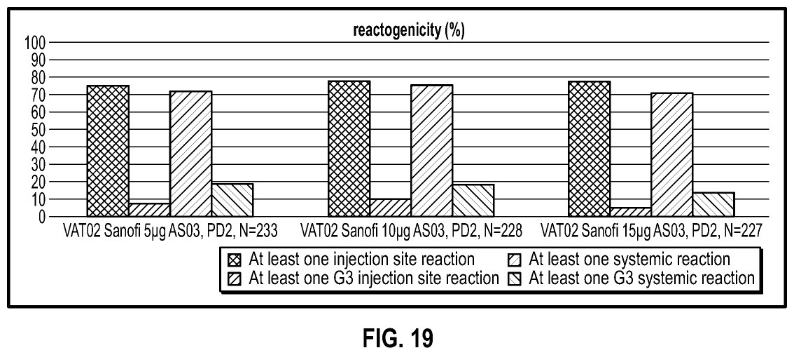

The safety data demonstrate similar safety profiles for the three treatment groups (5, 10, or 15 μg antigen+AS03). In the study, there were two immediate related reactions; but there were no adverse events of special interest (AESIs), no serious adverse events (SAEs), and no adverse events (AEs) leading to study discontinuation in the study. There were a limited number of Grade 3 unsolicited related reactions and related medically attended adverse events (MAAEs). Reactogenicity was similar in all treatment groups (FIG. 17). There was higher frequency and intensity of solicited reactions in the 18-59 years of age group compared to the ≥60 years of age group (FIG. 18). There also was increased frequency and intensity of solicited reactions post-injection 2 (FIG. 19).

The immunogenicity data show high seroconversion and responder rates in both young and older adults. Post-dose 2 (D36) responses in Per-Protocol Analysis Set (PPAS)-naïve D1+D22 show more than 95% seroconversion and responder rates in younger and older adults and no difference between the three treatment groups (Table 13; CI=confidence interval).

TABLE 13
Seroconversion Rates in All Age Groups

| | | Group 1 (5 μg + AS03) | | Group 2 (10 μg + AS03) | | Group 3 (15 μg + AS03) | |
|---|---|---|---|---|---|---|---|
| | | (N = 121) | | (N = 133) | | (N = 125) | |
| Age | | | % | | % | | % |
| (yrs) | Endpoint | n/M | (95% CI) | n/M | (95% CI) | n/M | (95% CI) |
| ≥18 | Responders | 114/116 | 98.3 | 123/124 | 99.2 | 115/118 | 97.5 |
| | (2-fold rise) | | (93.9; 99.8) | | (95.6; 100) | | (92.7; 99.5) |
| | Seroconversion | | 97.4 | | 97.6 | | 96.6 |
| | (4-fold rise) | | (92.6; 99.5) | | (93.1; 99.5) | | (91.5; 99.1) |
| 18-59 | Responders | 51/51 | 100 | 54/55 | 98.2 | 53/53 | 100 |
| | (2-fold rise) | | (93.0; 100) | | (90.3; 100) | | (93.3; 100) |
| | Seroconversion | | 100 | | 98.2 | | 100 |
| | (4-fold rise) | | (93.0; 100) | | (90.3; 100) | | (93.3; 100) |
| ≥60 | Responders | 63/65 | 96.9 | 69/69 | 100 | 62/65 | 95.4 |
| | (2-fold rise) | | (89.3; 99.6) | | (94.8; 100) | | (87.1; 99.0) |
| | Seroconversion | | 95.4 | | 97.1 | | 93.8 |
| | (4-fold rise) | | (87.1; 99.0) | | (89.9; 99.6) | | (85.0; 98.3) |

Antibody response data across all treatment groups, as indicated by post-dose 2 neutralizing antibody titers in Monogram PsVN assay in PPAS-naïve D1+D22 are shown in Table 14 below (“GMT”: geometric mean titer). In an exploratory analysis, neutralizing antibody titers were measured in a panel of human convalescent serum samples. Convalescent samples were obtained from 79 donors between days 17 and 47 after PCR-positive diagnosis of COVID-19. Donors had recovered (with clinical severity ranging from mild to severe) and were asymptomatic at the time of sample collection.

The data show that at the low dose group, the participants reached neutralizing antibody titers similar to those observed in convalescent sera. In the age group of 18-59 years, the GMT was nearly twice as high as that of the convalescent sera group.

TABLE 14
Antibody Responses Across Treatment Groups

| | Low Dose | | | Medium Dose | | | High Dose | | | | | |
|---|---|---|---|---|---|---|---|---|---|---|---|---|
| Age | (5 μg) | | | (10 μg) | | | (15 μg) | | | Convalescent Sera | | |
| (yrs) | N | GMT | 95% CI | N | GMT | 95% CI | N | GMT | 95% CI | N | GMT | 95% CI |
| ≥18 | 119 | 2100 | 1616; | 129 | 2374 | 1832; | 124 | 2483 | 1920; | 79 | 2140 | 1543; |
| | | | 2729 | | | 3076 | | | 3210 | | | 2967 |
| 18-59 | 53 | 2938 | 2155; | 58 | 3961 | 2795; | 54 | 4824 | 3637; | | | |
| | | | 4007 | | | 5612 | | | 6399 | | | |
| ≥60 | 66 | 1604 | 1079; | 71 | 1563 | 1098; | 70 | 1487 | 1037; | | | |
| | | | 2383 | | | 2223 | | | 2134 | | | |

The above data show that in naïve participants, seroconversion rates were >95% in both younger and older adults and there was no observed difference across treatment groups. The magnitude of antibody titers was higher in 18-59 year old versus those 60 years and older. No increase in magnitude was observed across treatment groups in 60 years and older group, nor 18 years and older. An increase was observed with higher antigen doses in the 18-59 years old group.

The CoV2 preS dTM AS03 composition also was evaluated in non-naïve participants. The data in Table 15 show that participants in the low dose group had a marked increase of neutralizing antibodies (as measured in (Monogram PsVN assay) after just one injection, with a GMT of 35,275 at Day 22. This GMT is more than 15-fold higher than the convalescent sera group shown in Table 14.

TABLE 15
Neutralizing Antibody in Non-Naive Population (FAS)

| | | Group 1 (5 μg + AS03) | | Group 2 (10 μg + AS03) | | Group 3 (15 μg + AS03) | |
|---|---|---|---|---|---|---|---|
| | Time | (N = 23) | | (N = 23) | | (N = 29) | |
| Age | point/ | | GMT/GMTR | | GMT/GMTR | | GMT/GMTR |
| group | ratio | M | (95% CI) | M | (95% CI) | M | (95% CI) |
| All | D 01 | 23 | 481 | 20 | 754 | 28 | 423 |
| | | | (259, 893) | | (297, 1912) | | (194, 924) |
| | D 22 | 23 | 18461 | 22 | 13824 | 26 | 12704 |
| | | | (8763, 38891) | | (5961, 32058) | | (3514, 45936) |
| | D 36 | 23 | 23460 | 21 | 21643 | 23 | 25067 |
| | | | (16912, 32543) | | (12139, 38587) | | (10104, 62190) |
| | D 22/D 01 | 23 | 38.4 | 19 | 19.4 | 25 | 38.2 |
| | | | (18.7, 79.0) | | (7.0, 53.5) | | (14.2, 103) |
| 18-59 | D 01 | 11 | 437 | 12 | 692 | 16 | 438 |
| yrs | | | (173, 1104) | | (149, 3204) | | (146, 1310) |
| | D 22 | 11 | 35275 | 13 | 15574 | 14 | 28499 |
| | | | (19407, 64116) | | (4730, 51285) | | (5527; 1.47E5) |
| | D 36 | 11 | 34645 | 12 | 33182 | 12 | 65532 |
| | | | (25055, 47905) | | (17621, 62487) | | (34384; 1.25E5) |
| | D 22/D 01 | 11 | 80.8 | 11 | 26.6 | 14 | 70.2 |
| | | | (28.9, 226) | | (5.8, 122) | | (16.0, 307) |
| ≥60 | D 01 | 12 | 526 | 8 | 858 | 12 | 405 |
| yrs | | | (198, 1392) | | (302, 2433) | | (111, 1480) |
| | D 22 | 12 | 10198 | 9 | 11637 | 12 | 4950 |
| | | | (2684, 38751) | | (2730, 49599) | | (564, 43404) |
| | D 36 | 12 | 16410 | 9 | 12242 | 11 | 8786 |
| | | | (9861, 27310) | | (4094, 36606) | | (1635, 47202) |
| | D 22/D 01 | 12 | 19.4 | 8 | 12.5 | 11 | 17.7 |
| | | | (7.3, 51.6) | | (2.5, 63.1) | | (4.4, 70.2) |

*GMTR: Geometric Mean Titer Ratios. FAS: full analysis set.

The data in non-naïve participants show that the magnitude of antibody titers was ≥5,000 in both the 18-59 years and >60 years age-groups after a single injection. No difference was observed across treatment groups.

These results show that CoV2 preS dTM AS03 triggered high neutralizing antibody responses in all adult age groups, including in those aged 60 years and older and those with prior infection. The immunogenic composition showed no safety concerns, with a well-tolerated safety profile across all three tested dose levels. The Phase II study showed 95% to 100% seroconversion following a second injection in all age groups and across all doses. In participants with previous evidence of SARS-COV-2 infection, significantly higher antibody responses were generated after a single vaccine dose, suggesting strong potential for development as a booster vaccine.

The Phase 2 data confirm the potential of the CoV2 preS dTM AS03 immunogenic composition to play a role in addressing a global public health crisis, as it is well recognized that multiple vaccines will be needed, especially as variants continue to emerge as well as the need for booster vaccines. Based on these positive Phase 2 results, the 10 μg dose level will be further evaluated in a global Phase 3 study in more than 35,000 participants.

### Example 11: Immunogenicity of Variant Monovalent (B.1.351) and Bivalent (D614+B.1.351) Vaccines in Non-Human Primates

An immunogenicity study (CoV2-06_NHP) was performed in naïve non-human primates (NHPs) to assess the immunogenicity of the variant monovalent vaccine (B.1.351) and potential negative interferences when combined with D614 preS dTM antigen in the bivalent formulation (D614+B.1.351), in the presence of AS03. At the peak of the antibody responses (Day 34), the neutralizing antibody titers were measured against the two viruses (D614 and Beta), as well as other variants of concerns (VoCs), Alpha, Gamma, and Delta. The neutralization titers against the D614 strain (obtained in a VSV-pseudovirus qualified assay from Nexelis) were compared between monovalent and bivalent formulations to evaluate the impact of each component in the bivalent formulation on the immunogenicity of the other component.

### Study Design

Fifty-four adult Mauritian cynomolgus macaques (males and females) of two to eight years old were randomized for age, weight, and sex. Nine groups of six macaques were immunized with the three vaccine formulations (D614, B.1.351, and bivalent) at doses of 2.5, 5, and 10 μg per component, in the presence of AS03 (Table 16). Two injections at three weeks apart were given by the IM route in the deltoid, same side for both administrations, under 0.5 mL doses.

TABLE 16
CoV2-06_NHP Study Groups

| | CoV2 preS | D614 | B.1.351 | |
|---|---|---|---|---|
| Groups | dTM antigen | dose (μg) | dose (μg) | Adjuvant |
| 1 | D614, 2.5 μg | 2.5 | — | AS03 |
| 2 | D614, 5 μg | 5 | — | AS03 |
| 3 | D614, 10 μg | 10 | — | AS03 |
| 4 | B.1.351, 2.5 μg | — | 2.5 | AS03 |
| 5 | B.1.351, 5 μg | — | 5 | AS03 |
| 6 | B.1.351, 10 μg | — | 10 | AS03 |
| 7 | Bivalent, 2.5 μg | 2.5 | 2.5 | AS03 |
| 8 | Bivalent, 5 μg | 5 | 5 | AS03 |
| 9 | Bivalent, 10 μg | 10 | 10 | AS03 |

Animals were monitored throughout the study for any clinical signs of adverse events. Blood samples were collected pre-study, and on D2, D21, D23, and D34 for blood cell count and chemistry parameters. Blood samples were collected on D21 and on D34 for antibody titration. Binding IgG levels were measured in ELISA against the GCN4-Spike protein, and the neutralization antibodies were measured on D34 against the D614, D614G, Alpha, Beta, Gamma, and Delta variants using PsV neutralizing assay (SP REI Cambridge). In parallel, the samples were titrated in a qualified D614 PsV neutralization assay (Nexelis) to quantify the interferences on D614 titers. The characteristics of the two neutralization assays used are shown in Table 17 below.

TABLE 17
Neutralization Assays Used for CoV2-06_NHP Study

| Assay | Nexelis | SP REI Cambridge |
|---|---|---|
| Virus | VSV-PsV | Lentivirus-PsV |
| Cell | Vero | 293-ACE2 |
| Qualified | Yes | No |
| Strain(s) | D614 | D614G, Alpha, Gamma, Beta, Delta |

### Results

No clinical signs were observed during the study, and hematology and clinical chemistry parameters as well as body temperature did not show any concerning safety signal. Three weeks after the first injection (D21), all macaques mounted S-binding antibodies as measured by ELISA, except for one macaque in the 2.5 μg B.1.351 monovalent vaccine group. Mean ELISA titers ranged from 3.5 to 4.1 log₁₀ EU (data not shown). Two weeks after the second injection (D34), S-binding antibody titers increased in all groups compared to D21 and ranged from 4.9 to 5.4 Log₁₀ EU. No statistically significant differences were seen on ELISA titers between the doses and vaccine formulations.

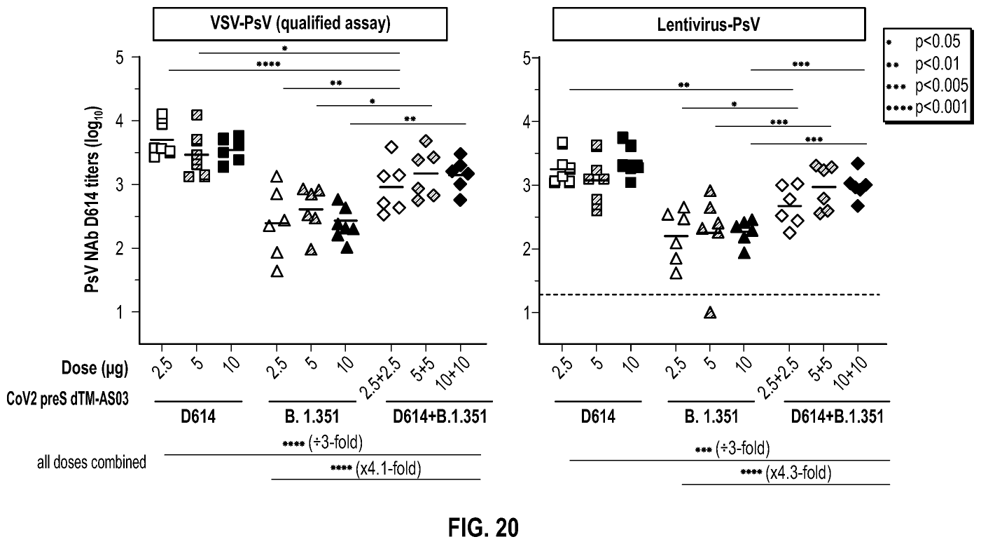

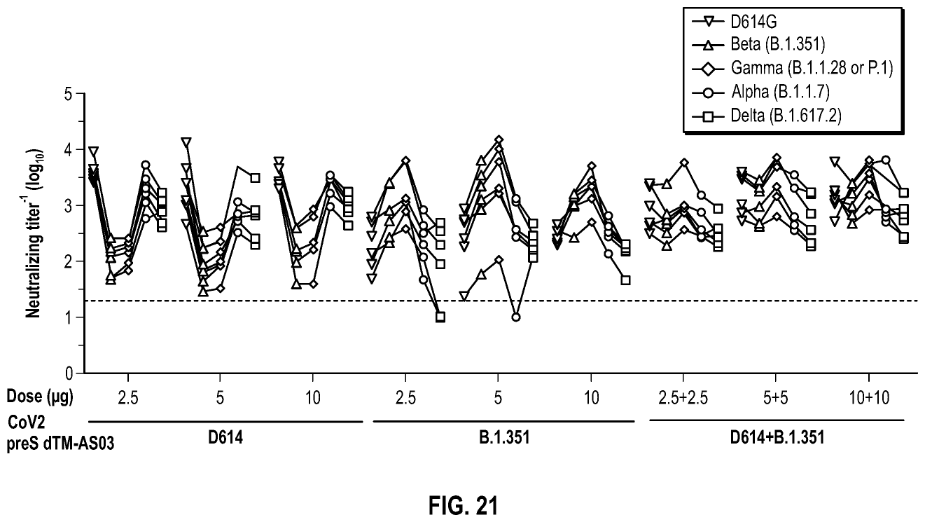

Two weeks after the second injection (D34), neutralizing antibodies (NAb) were measured against the parental strain D614 in both neutralization assays (qualified VSV-PsV and lentivirus-PsV) (FIG. 20) and against the parental strain D614G, Alpha, Beta, Gamma and Delta variants in the lentivirus-PsV assay (FIG. 21).

D614 VSV-PsV NAb titers were detected in all macaques on D34 at various levels depending on the vaccine formulation. No dose-effect was observed for the three formulations (monovalent D614 and B.1.351 formulations and bivalent D614+B.1.351 formulation) from 2.5 to 10 μg per component. D614 VSV-PsV neutralizing titers were highest in the monovalent D614 vaccine groups with a mean titer of 3.6 Log₁₀ for all three dose levels, lowest in the B.1.351 monovalent groups with a mean titer of 2.5 Logo, and intermediate in the bivalent vaccine groups with a mean titer of 3.1 Log₁₀. When bivalent and monovalent D614 vaccines were compared at the same dose per component, the difference on D614 NAb titers was statistically significant at the 2.5 μg dose level (monovalent D614 2.5 μg and bivalent 2.5 μg+2.5 μg) (5.4-fold, p-value<0.001). The differences were lower (2-fold) and not statistically significant at the two other dose levels (5 μg and 10 μg of D614 component).

In contrast, the increase of D614 VSV-PsV NAb titers in the bivalent vaccine compared to the monovalent B.1.351 vaccine was statistically significant at all dose levels (3.6 to 5.3-fold, p-values<0.05). When all three dose levels were combined, the decrease in D614 VSV-PsV neutralizing titers observed with the bivalent vaccine was 3-fold compared to titers in the monovalent D614 vaccine groups (p-value<0.001), but they were 4.1-fold higher than titers induced by the monovalent B.1.351 vaccine (p-value<0.001).

The moderate decrease on D614 NAb titers observed in the VSV-PsV assay with the bivalent vaccine compared to the monovalent D614 vaccine was confirmed in the lentivirus-PsV neutralization assay at the 2.5 μg dose level (3.8-fold, p-value<0.01) and for all three doses combined (2.3-fold p-value<0.005). Similar to the results with the VSV-PsV assay, the increase on D614 NAb titers compared to the monovalent B.1.351 vaccine was confirmed at all three doses levels (2.9- to 5.3-fold, p-values<0.01) and on all three doses combined (4.3-fold, p-value<0.001) in the lentivirus-PsV assay. One animal in the monovalent B.1.351 5 μg group had undetectable D614 NAb titer, and this same animal had a low titer against D614 in the VSV-PsV assay and low titers against D614G and all VoCs.

The neutralization of the Beta variant and the other known VoCs (Alpha, Gamma and Delta) was also assessed in the lentivirus-PsV neutralizing assay. NAb titers against Alpha and Delta variants followed the same pattern as those against D614G for each vaccine formulation: highest for monovalent D614 vaccine, lowest for monovalent B.1.351 vaccine, and intermediate for the bivalent vaccine. The titers against Alpha and Delta variants were only slightly lower than those against the D614G strain (similar to 2.5-fold lower and 1.2 to 4.9-fold lower, respectively). Titers against Beta and Gamma variants were lowest for the monovalent D614 vaccine and highest for the monovalent B.1.351 vaccine. The bivalent vaccine induced Beta and Gamma NAb titers at the same level as the monovalent B. 1.351 vaccine.

Overall, compared to monovalent D614 vaccine, the bivalent vaccine induced slightly lower NAb titers against the parental strain (2 to 3-fold depending on the assay), much higher titers against the Beta and Gamma variants, and comparable neutralization of the two most widely circulating variants Alpha and Delta. Compared to the monovalent B. 1.351 vaccine, the bivalent vaccine induced much higher NAb titers against the parental D614 strain and the D614G strain, as well as against Alpha and Delta variants (Table 18).

TABLE 18
Fold-Change on NAb Titers in Bivalent vs. Monovalent Vaccines*

| | Bivalent vs. | Bivalent vs. |
|---|---|---|
| NAb Titers Against | Monovalent D614 | Monovalent B.1.351 |
| D614 (qualified assay) | ÷3 (p < 0.001) [symbol] | ×4.1 (p < 0.001) [symbol] |
| D614 | ÷2.3 (p < 0.005) [symbol] | ×4.3 (p < 0.001) [symbol] |
| D614G | ÷2.3 (p < 0.01) [symbol] | ×5.3 (p < 0.001) [symbol] |
| B.1.1.7 (Alpha) | ÷1.9 (NS) [symbol] | ×2.9 (p < 0.05) [symbol] |
| B.1.351 (Beta) | ×7.8 (p < 0.001) [symbol] | ÷1.1 (NS) [symbol] |
| B.1.1.28 (Gamma) | ×12.8 (p < 0.001) [symbol] | 1 (NS) [symbol] |
| B.1.617.2 (Delta) | ÷1.6 (NS) [symbol] | ×3.6 (p < 0.001) [symbol] |

*All doses combined.
÷fold-decrease,
×fold-increase,
p: p-values,
NS: non-statistically significant.
NAb titers assayed using the lentivirus-based PsV assay unless specified.

In conclusion, these data generated in naïve NHPs showed a balanced neutralization of all variants of concern known to date. The bivalent vaccine (D614+B.1.351) warrants further clinical investigation for primary vaccination as it may mitigate the risk of vaccine escape, in view of the highly dynamic epidemiology. Indeed, with the increasing seroprevalence of D614-like Spikes due to vaccination and natural infection with Alpha and Delta variants, vaccine escape variants such as Beta and Gamma might become dominant in the future, especially if mutations linked to antibody escape (such as E484K) combine with mutations linked to increased transmissibility, as present in the Delta variant.

### Example 12: NHP Variant-Booster Studies

This Example describes studies (CoV2-07_NHP and CoV2-08_NHP) that evaluated the use of the monovalent and bivalent CoV2 preS dTM-AS03 vaccines (D614, B.1.351, or D614+B.1.351) as a booster after a primary vaccination with different vaccine platforms. Although the benefit of AS03 was clearly demonstrated in the primary vaccination to induce a robust immune response, the role of AS03 in potentiating the immune response was evaluated here to determine whether an adjuvant would be useful in a booster regimen.

Booster immunization was evaluated in cynomolgus macaques immunized with COVID-19 mRNA-LNP vaccine candidates (CoV2-07_NHP, mRNA-primed cohort) and in rhesus macaques immunized with CoV2 preS dTM-AS03 vaccine (CoV2-08_NHP, subunit-primed cohort: Wuhan strain). Both cohorts received the booster injection about 7 months after the primary vaccination.

### Study Design

In the CoV2-07_NHP study, 16 Mauritian cynomolgus macaques (eight males and eight females, four to ten years of age), previously vaccinated with COVID-19 mRNA-LNP vaccine candidates (Kalnin et al., NPJ Vaccines (2021) 6(1):61), were randomized into four groups of four macaques. Only animals that developed a neutralizing antibody response against the parental D614 virus two weeks after the primary vaccination (D35) were selected for the study.

In the CoV2-08-NHP study, 24 Indian rhesus macaques (males, 2.7 to 4 years of age), previously immunized with CoV2 preS dTM-AS03 (Wuhan strain), were randomized into five groups of four to five macaques.

In both cohorts, randomization was based on baseline characteristics (sex and age) and the neutralizing responses post-primary immunization as well as one month before the booster vaccination. The different groups received one dose of the CoV2 preS dTM vaccine formulations as described in Table 19.

TABLE 19
NHP Booster Study Groups

| Cohort (Study) | Group | N | Vaccine antigen | Total Dose | Adjuvant |
|---|---|---|---|---|---|
| mRNA-Primed | 1 | 4 | B.1.351 | 5 | — |
| (CoV2-07_NHP) | 2 | 4 | D614 | 5 | AS03 |
| | 3 | 4 | B.1.351 | 5 | AS03 |
| | 4 | 4 | D614 + B.1.351 | 5 (2.5 + 2.5) | AS03 |
| Subunit-Primed | 1 | 4 | B.1.351 | 5 | — |
| (CoV-08_NHP) | 2 | 5 | D614 | 5 | AS03 |
| | 3 | 5 | B.1.351 | 5 | AS03 |
| | 4 | 5 | D614 + B.1.351 | 10 (5 + 5) | AS03 |
| | 5 | 5 | D614 + B.1.351 | 5 (2.5 + 2.5) | AS03 |

Animals were monitored throughout the study for any clinical signs of adverse events. Blood samples were collected before booster injection, on D2, D7, D14, D21, and D28 post-injection for blood cell count and chemistry parameters. Blood samples were collected on D7, D14, D21 and D28 for binding and lentivirus-PsV neutralizing antibody titration on SARS-COV-2_{D}614G, Alpha, Beta, Gamma, and Delta variants.

### Results

No clinical signs were observed during the study, and hematology and clinical chemistry parameters as well as body temperature did not show any concerning safety signal.

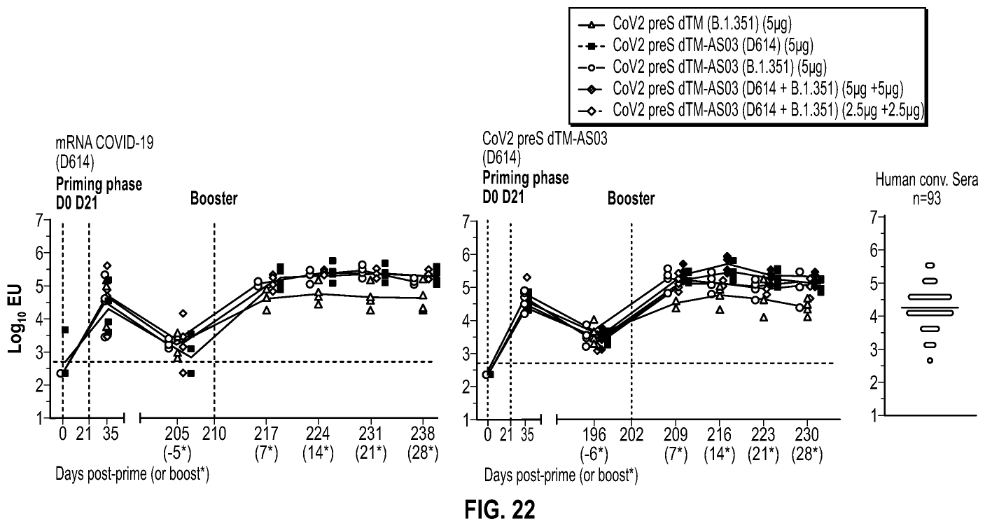

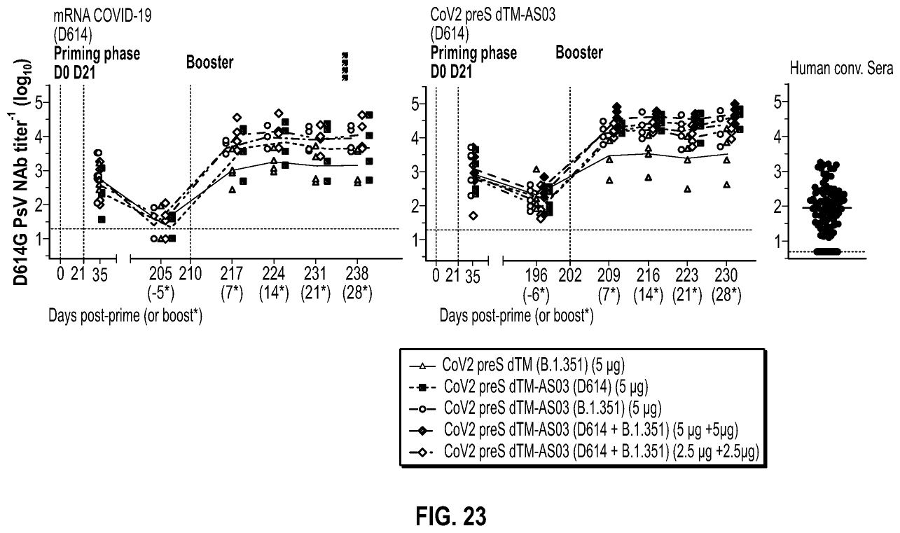

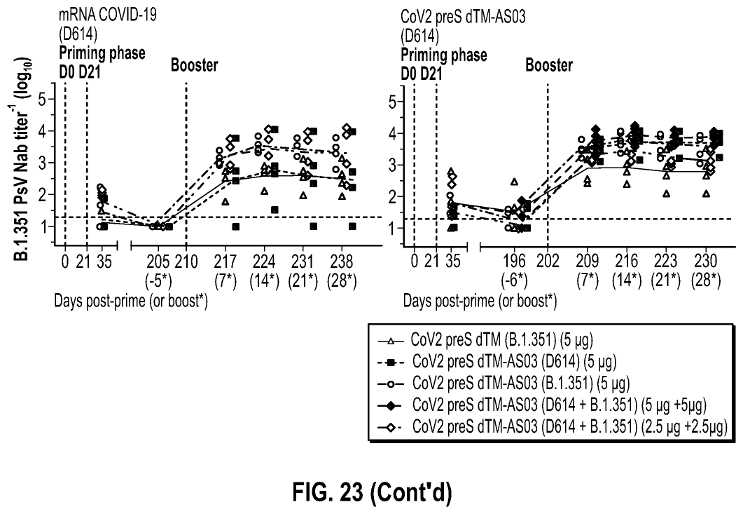

The S-binding IgG titers and NAb titers against the D614G strain and B. 1.351 variant were measured weekly for four weeks after the booster, and compared to the peak titers post-primary vaccination (D35) or at baseline, i.e., seven months post-primary immunization (D205 in the mRNA-primed cohort and D196 in the subunit-primed cohort, respectively). The S-binding IgG titers are shown in FIGS. 22 and D614G and B.1.351 NAb titers are shown in FIG. 23. S binding and D614 NAb titers measured in human convalescent sera are represented on the corresponding figures.

Seven months post-primary vaccination (D205 or D196, representing the baseline), S-binding titers had declined in both cohorts but were still detected in most animals. The booster immunization increased the S-binding titers in all animals as soon as D7 regardless of the vaccine formulation. The increase was more prominent in the presence of AS03 (3.5-fold, non-statistically significant in the mRNA-primed cohort and 5.7-fold, p<0.05 in the subunit-primed cohort). The high IgG titers were stable between D7 and D28, the final timepoint analyzed.

Consistent with the S-binding titers, the D614G NAb titers had declined seven months post-primary vaccination (D205 or D196) in both cohorts, and some animals from the mRNA-primed cohort had negative titers. At this timepoint, the B.1.351 NAb titers were all undetectable in the mRNA-primed cohort and undetectable or low in the subunit-primed cohort.

One week after the booster, the NAb titers strongly increased against both strains (D614G and B.1.351) in all vaccinated groups from both cohorts, except in one animal from the mRNA-primed cohort boosted with the AS03-adjuvanted monovalent D614 (B.1.351 NAb titers were detected on D14 only while D614G NAb titers were in the same range as the other animals). Importantly, the increase was stable for at least 4 weeks (final timepoint tested) in all groups and in both cohorts.

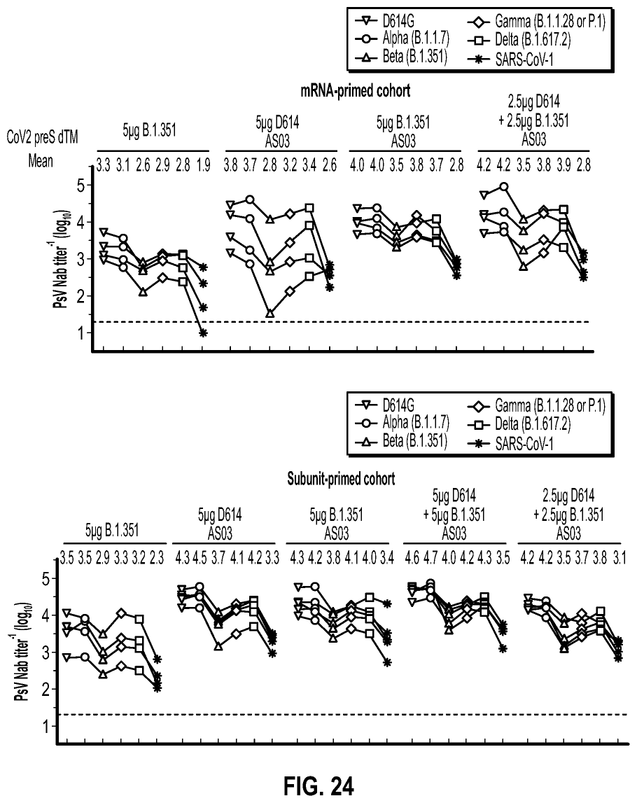

To explore the breadth of the neutralizing response, NAb titers against other known VoCs (Alpha, Gamma, and Delta) and SARS-COV-I was analyzed two weeks post-booster and compared to NAb titers against D614G and B.1.351 at the same time point (FIG. 24). Confirming the results on the two prototype strains (D614G and B.1.351), high NAb titers against the other variants were measured after the booster immunization with all vaccine formulations.

The data indicate that, in vaccine-primed macaques, a third injection with the various vaccine formulations (unadjuvanted monovalent B.1.351, AS03-adjuvanted monovalent D614 or B.1.351, or bivalent) induced a strong recall of the initial NAb response against the parental D614 strain, extended the neutralization to the Beta variant and all other known VoCs (Alpha, Gamma, and Delta) as well as to SARS-COV-1.

The AS03-adjuvanted formulations induced higher NAb titers compared to the unadjuvanted monovalent (statistically significant on the D614G NAb titer increase vs. baseline—6.5-fold and 8.1-fold in the mRNA-primed and subunit-primed cohorts, respectively).

A trend for higher responses against Beta and Gamma variants was observed with vaccines containing the B.1.351 S antigen (monovalent or bivalent), with mean NAb titers against VoCs greater than 3.5 Log₁₀ in both cohorts two weeks post-booster and mean NAb titers against D614G greater than 4.0 Log₁₀. No interference (negative or positive) on NAb responses were observed in the bivalent vaccine compared to monovalent D614 or B.1.351.

Importantly, the Ab titers measured weekly post-booster (S-binding and NAbs against D614G and Beta) appear to be stable from D7 to D28, suggesting that they reached a plateau as early as D7. The observations were reproduced in macaques immunized with different vaccine platforms (mRNA and subunit), despite low to undetectable NAb responses against variants at the time of the booster.

LIST OF SEQUENCES

| SEQ | |
|---|---|
| ID | |
| NO | Description |
| 1 | SARS-COV-2 S protein amino acid sequence (Wuhan) |
| 2 | SARS-COV-2 S protein signal peptide amino acid sequence |
| 3 | Mutant chitinase signal peptide amino acid sequence |
| 4 | SARS-COV-2 S protein ectodomain amino acid sequence |
| 5 | RRAR |
| 6 | GSAS |
| 7 | Foldon amino acid sequence |
| 8 | Wildtype foldon coding sequence |
| 9 | Codon-optimized foldon coding sequence |
| 10 | preS dTM amino acid sequence |
| 11 | Wildtype chitinase signal peptide amino acid sequence |
| 12 | Polyhedrin promoter burst sequence |
| 13 | Recombinant S protein amino acid sequence derived from |
| | B.1.351 |
| 14 | gBlock pPSC12-DB 3′ fragment sequence |
| 15 | F1-gBlock-5′ fragment sequence |
| 16 | F1-gBlock-3′ fragment sequence |
| 17 | F2-gBlock-5′ fragment sequence |
| 18 | F2-gBlock-3′ fragment sequence |
| 19 | F3-gBlock-5′ fragment sequence |
| 20 | F3-gBlock-3′ fragment sequence |
| 21 | gBlock pPSC12-DB 5′ fragment 1 sequence |
| 22 | F3b-gBlock-3′ fragment sequence |
| 23 | gBlock pPSC12-DB 5′ fragment 2 sequence |

1. An immunogenic composition comprising
(a) one, two, three, or more recombinant SARS-COV-2 proteins, wherein one or more of the proteins is a trimer of a polypeptide comprising, from N terminus to C terminus,
(i) a sequence that is at least 95% identical to residues 19-1243 of SEQ ID NO:10, wherein residues GSAS (SEQ ID NO:6) at positions 687-690 of SEQ ID NO:10 and residues PP at positions 991 and 992 of SEQ ID NO: 10 are maintained in the sequence; and
(ii) a trimerization domain, wherein the trimerization domain comprises SEQ ID NO:7; and
(b) an adjuvant, wherein the adjuvant is an oil-in-water emulsion comprising tocopherol and squalene.

2. An immunogenic composition comprising
(a) one, two, three, or more recombinant SARS-COV-2 S proteins, each produced by a method comprising
introducing into insect cells a baculoviral vector for expressing a polypeptide comprising, from N terminus to C terminus, (i) a signal peptide derived from an insect or baculoviral protein, (ii) a sequence that is at least 95% identical to residues 19-1243 of SEQ ID NO:10, wherein residues GSAS (SEQ ID NO:6) at positions 687-690 of SEQ ID NO:10 and residues PP at positions 991 and 992 of SEQ ID NO:10 are maintained in the sequence; and (iii) a trimerization domain, wherein the trimerization domain comprises SEQ ID NO:7,
culturing the insect cells under conditions that allow expression and trimerization of the polypeptide, and
isolating the recombinant SARS-COV-2 S protein from the culture, wherein the recombinant SARS-COV-2 S protein is a trimer of the polypeptide, without the signal sequence; and
(b) an adjuvant, wherein the adjuvant is an oil-in-water emulsion comprising tocopherol and squalene.

3. The immunogenic composition of claim 2, wherein the baculoviral vector comprises a polyhedrin promoter linked operably to a coding sequence for the polypeptide.

4. The immunogenic composition of claim 2, wherein the insect or baculoviral protein is a chitinase.

5. The immunogenic composition of claim 4, wherein the signal peptide comprises SEQ ID NO:3.

6. The immunogenic composition of claim 1, wherein composition comprises
(i) a recombinant SARS-COV-2 protein that is a trimer of a polypeptide comprising or having a sequence identical to residues 19-1243 of SEQ ID NO:10,
(ii) a recombinant SARS-COV-2 protein that is a trimer of a polypeptide comprising or having a sequence identical to residues 19-1240 of SEQ ID NO:13, or
(iii) both (i) and (ii), optionally in equal amounts.

7. The immunogenic composition of claim 1, wherein for every 0.5 mL or each dose of the composition, the composition comprises or is prepared by mixing:
(i) an antigen component comprising about 2 μg to about 50 μg, optionally about 2.5 μg to about 50 μg or about 5 μg to about 50 μg, of each of the recombinant SARS-COV-2 S protein(s); and
(ii) an oil-in-water emulsion adjuvant comprising or prepared by mixing
a) 10.69 mg squalene, 4.86 mg polysorbate 80, and 11.86 mg α-tocopherol in a phosphate-buffered saline, optionally as shown in Table 9, further optionally wherein the adjuvant is provided in 0.25 mL
b) 5.35 mg squalene, 2.43 mg polysorbate 80 and 5.93 mg α-tocopherol in a phosphate-buffered saline, optionally as shown in Table 9, further optionally wherein the adjuvant is provided in 0.25 mL,
c) 2.67 mg squalene, 1.22 mg polysorbate 80 and 2.97 mg α-tocopherol in a phosphate-buffered saline, optionally as shown in Table 9, further optionally wherein the adjuvant is provided in 0.25 mL, or
d) 1.34 mg squalene, 0.61 mg polysorbate 80 and 1.48 mg α-tocopherol in a phosphate-buffered saline, optionally as shown in Table 9, further optionally wherein the adjuvant is provided 0.25 mL.

8. The immunogenic composition of claim 7, wherein for every 0.5 mL or each dose of the composition, the composition comprises 2.5, 5, 10, 15, or 45 μg of each of the recombinant SARS-COV-2 S protein(s).

9. The immunogenic composition of claim 7, wherein the antigen component comprises or is prepared by mixing:
0.097 mg sodium phosphate monobasic monohydrate,
0.26 mg sodium phosphate dibasic anhydrous,
2.2 mg sodium chloride,
550 μg polysorbate 20, and
about 0.25 mL water.

10. The immunogenic composition of claim 1, wherein for every 0.5 mL or each dose of the composition, the composition comprises a total of 5 μg of the recombinant SARS-COV-2 S protein(s), optionally wherein the composition comprises two different recombinant SARS-COV-2 S proteins in equal amounts.

11. The immunogenic composition of claim 1, wherein for every 0.5 mL or each dose of the composition, the composition comprises a total of 10 μg of the recombinant SARS-COV-2 S protein(s), optionally wherein the composition comprises two different recombinant SARS-COV-2 S proteins in equal amounts.

12. A container containing the immunogenic composition of claim 1.

13. The container of claim 12, wherein the container is a vial or a syringe.

14. The container of claim 12, wherein the container contains a single dose of the immunogenic composition, optionally the single dose is in a volume of 0.5 mL.

15. The container of claim 12, wherein the container contains multiple doses of the immunogenic composition, optionally each of the doses is in a volume of 0.5 mL.

16. A kit for intramuscular vaccination, wherein the kit comprises two containers, wherein
(a) a first container contains a pharmaceutical composition comprising one, two, three, or more recombinant SARS-COV-2 S proteins, wherein one or more of the proteins is a trimer of a polypeptide comprising, from N terminus to C terminus,
(i) a sequence that is at least 95% identical to
(A) residues 19-1243 of SEQ ID NO:10, wherein residues GSAS (SEQ ID NO:6) at positions 687-690 of SEQ ID NO: 10 and residues PP at positions 991 and 992 of SEQ ID NO:10 are maintained in the sequence, or
(B) residues 19-1240 of SEQ ID NO:13, wherein residues GSAS (SEQ ID NO:6) at positions 684-687 of SEQ ID NO: 13 and residues PP at positions 988 and 989 of SEQ ID NO: 13 are maintained in the sequence; and
(ii) a trimerization domain, wherein the trimerization domain comprises SEQ ID NO:7; and
(b) a second container contains an oil-in-water adjuvant comprising tocopherol and squalene.

17. The kit of claim 16, wherein the first container comprises
(i) a recombinant SARS-COV-2 protein that is a trimer of a polypeptide comprising or having a sequence identical to residues 19-1243 of SEQ ID NO:10,
(ii) a recombinant SARS-COV-2 protein that is a trimer of a polypeptide comprising or having a sequence identical to residues 19-1240 of SEQ ID NO:13, or
(iii) both (i) and (ii), optionally in equal amounts.

18. The kit of claim 16, wherein the first container comprises one or more doses of the recombinant SARS-COV-2 S protein(s), wherein each dose of the protein(s) is a total of about 2.5 to 45, optionally 5 to 45, μg provided in 0.25 mL of a phosphate-buffered saline, optionally as shown in Table A, Table 8 or Table 12.

19. The kit of claim 18, wherein each dose of the protein is a total of 2.5, 5, 10, 15, or 45 μg.

20. The kit of claim 16, wherein the second container comprises one or more doses of the adjuvant, wherein each dose of the adjuvant is 0.25 mL in volume and comprises
(a) 10.69 mg squalene,
4.86 mg polysorbate 80, and
11.86 mg α-tocopherol, in a phosphate-buffered saline, optionally as shown in Table 9,
(b) 5.35 mg squalene,
2.43 mg polysorbate 80, and
5.93 mg α-tocopherol, in a phosphate-buffered saline, optionally as shown in Table 9,
(c) 2.67 mg squalene,
1.22 mg polysorbate 80, and
2.97 mg α-tocopherol, in a phosphate-buffered saline, optionally as shown in Table 9,
or
(d) 1.34 mg squalene,
0.61 mg polysorbate 80, and
1.48 mg α-tocopherol, in a phosphate-buffered saline, optionally as shown in Table 9.

21. A method of making a vaccine kit, comprising:
providing the recombinant S protein(s) and the adjuvant of the immunogenic composition of claim 1, and
packaging the protein and the adjuvant into separate sterile containers.

22. A method of preventing or ameliorating COVID-19 in a subject in need thereof, comprising administering to the subject a prophylactically effective amount of the immunogenic composition of claim 1.

23. A method of preventing or ameliorating COVID-19 in a subject in need thereof, comprising administering to the subject a prophylactically effective amount of the immunogenic composition of claim 1, wherein prior to the administering step, the subject has been infected by SARS-COV-2 or has been vaccinated with a first COVID-19 vaccine.

24. The method of claim 23, wherein prior to the administering step, the subject has been vaccinated with a genetic, subunit, or killed vaccine.

25. The method of claim 24, wherein the subject has been vaccinated with a genetic vaccine comprising an mRNA that encodes a recombinant SARS-COV-2 S antigen.

26. The method of claim 23, wherein the administering step takes place 4 weeks, one month, two months, three months, four months, five months, six months, seven months, eight months, nine months, ten months, eleven months, one year, or more, optionally four to ten months, further optionally eight months, post-infection or after the subject is vaccinated with the first COVID-19 vaccine, optionally wherein the immunogenic composition comprises 2.5 or 5 μg of each of the recombinant SARS-COV-2 S protein(s), and further optionally wherein the immunogenic composition is monovalent or multivalent.

27. The method of claim 22, wherein the immunogenic composition is administered intramuscularly to the subject at about 5 to about 50 μg, or about 2.5 to about 50 μg, optionally 2.5, 5, 10, 15, or 45 μg, of the recombinant SARS-CoV-2 S protein(s) per dose.

28. The method of claim 22, wherein the prophylactically effective amount is administered in a single dose or in two or more doses, optionally intramuscularly.

29. The method of claim 28, comprising administering to the subject two doses of the immunogenic composition with an interval of about two weeks to about three or four months, wherein each dose of the immunogenic composition comprises a total of 2.5, 5 or 10 μg of the recombinant SARS-COV-2 S protein(s).

30. The method of claim 29, wherein the interval is about three weeks or about 21 days, or about four weeks or about 28 days, or about one month.

31. The method of claim 22, wherein the subject is a human subject, optionally wherein the human subject is a child, an adult, or an elderly adult.

32-33. (canceled)

34. The immunogenic composition of claim 1, wherein the tocopherol is alpha-tocopherol, optionally D,L-alpha-tocopherol.

35. The immunogenic composition of claim 1, wherein the adjuvant has an average droplet size of less than 1 μm, optionally wherein the average droplet size is less than 500 nm, less than 200 nm, 50 to 200 nm, 120 to 180 nm, or 140 to 180 nm.

36. The immunogenic composition of claim 1, wherein the adjuvant has a polydispersity index of 0.5 or less, or 0.3 or less, such as 0.2 or less.

37. The immunogenic composition of claim 1, wherein the adjuvant comprises a surfactant selected from poloxamer 401, poloxamer 188, polysorbate 80, sorbitan trioleate, sorbitan monooleate and polyoxyethylene 12 cetyl/stearyl ether, either alone, or in combination with each other or in combination with other surfactants.

38. The immunogenic composition of claim 1, wherein the adjuvant comprises a surfactant selected from polysorbate 80, sorbitan trioleate, sorbitan monooleate, and polyoxyethylene 12 cetyl/stearyl ether either alone, or in combination with each other.

39. The immunogenic composition of claim 1, wherein the adjuvant comprises polysorbate 80.

40. The immunogenic composition of claim 1, wherein adjuvant comprises one, two, or three surfactants.

41. The immunogenic composition of claim 1, wherein the weight ratio of squalene to tocopherol in the adjuvant is 0.1 to 10, optionally 0.2 to 5, 0.3 to 3, 0.4 to 2, 0.72 to 1.136, 0.8 to 1, 0.85 to 0.95, or 0.9.

42. The immunogenic composition of claim 1, wherein the weight ratio of squalene to surfactant in the adjuvant is 0.73 to 6.6, optionally 1 to 5, 1.2 to 4, 1.71 to 2.8, 2 to 2.4, 2.1 to 2.3, or 2.2.

43. The immunogenic composition of claim 1, wherein the amount of squalene in a single dose of the adjuvant is at least 1.2 mg, optionally 1.2 to 20 mg, 1.2 to 15 mg, 1.2 to 2 mg, 1.21 to 1.52 mg, 2 to 4 mg, 2.43 to 3.03 mg, 4 to 8 mg, 4.87 to 6.05 mg, 8 to 12.1 mg, or 9.75 to 12.1 mg.

44. The immunogenic composition of claim 1, wherein the amount of tocopherol in a single dose of the adjuvant is at least 1.3 mg, optionally 1.3 to 22 mg, 1.3 to 16.6 mg, 1.3 to 2 mg, 1.33 to 1.69 mg, 2 to 4 mg, 2.66 to 3.39 mg, 4 to 8 mg, 5.32 to 6.77 mg, 8 to 13.6 mg, or 10.65 to 13.53 mg.

45. The immunogenic composition of claim 1, wherein the amount of surfactant in a single dose of the adjuvant is at least 0.4 mg, optionally 0.4 to 9.5 mg, 0.4 to 7 mg, 0.4 to 1 mg, 0.54 to 0.71 mg, 1 to 2 mg, 1.08 to 1.42 mg, 2 to 4 mg, 2.16 to 2.84 mg, 4 to 7 mg, or 4.32 to 5.68 mg.

46. The immunogenic composition of claim 1, wherein the adjuvant comprises or consists essentially of
squalene,
tocopherol, optionally D,L-alpha-tocopherol
surfactant, optionally polysorbate 80, and
water.

47. The immunogenic composition of claim 1, wherein the volume of a single dose for intramuscular injection is 0.05 to 1 mL, optionally 0.1 to 0.6 mL, 0.2 to 0.3 mL, 0.25 mL, 0.4 to 0.6 mL, or 0.5 mL.

48. The immunogenic composition of claim 1, wherein the immunogenic composition, or a mixture of the contents of the first and second containers, has a pH of 4 to 9, optionally 5 to 8.5, 5.5 to 8, or 6.5 to 7.4.

49. The immunogenic composition of claim 1, wherein the immunogenic composition, or a mixture of the contents of the first and second containers, has an osmolality of 250 to 750 mOsm/kg, optionally 250 to 550 mOsm/kg, 270 to 500 mOsm/kg, or 270 to 400 mOsm/kg.

50. The immunogenic composition of claim 1, wherein the immunogenic composition, or a mixture of the contents of the first and second containers, comprises squalene at 0.8 to 100 mg per mL, optionally 1.2 to 48.4 mg per mL, 10 to 30 mg per mL, or 21.38 mg per mL.

51. The immunogenic composition of claim 1, wherein the volume of a single dose of the adjuvant for intramuscular injection, prior to being mixed with the recombinant S protein, is 0.05 mL to 1 mL, optionally 0.1 to 0.6 mL, 0.2 to 0.3 mL, 0.25 mL, 0.4 to 0.6 mL, or 0.5 mL.

52. The immunogenic composition of claim 1, wherein the adjuvant, prior to being mixed with the recombinant S protein,
has a pH of 4 to 9, optionally 5 to 8.5, 5.5 to 8, or 6.5 to 7.4,
has an osmolality of 250 to 750 mOsm/kg, optionally 250 to 550 mOsm/kg, 270 to 500 mOsm/kg, or 270 to 400 mOsm/kg,
comprises a buffer and/or tonicity modifying agents, optionally a modified phosphate-buffered saline, optionally as shown in Table 9
has a squalene concentration of 0.8 to 100 mg per mL, optionally 1.2 to 48.4 mg per mL, and
has a single dose volume of 0.05 mL to 1 mL, optionally 0.1 to 0.6 mL.

53. The immunogenic composition of claim 1, wherein the recombinant S protein is provided in an aqueous liquid solution that has, prior to being mixed with the adjuvant,
a single dose volume of 0.2 to 0.3 mL, optionally 0.25 mL, 0.4 to 0.6 mL, or 0.5 mL,
a pH of 4 to 9, optionally 5 to 8.5, 5.5 to 8, or 6.5 to 7.4, and
an osmolality of 250 to 750 mOsm/kg, optionally 250 to 550 mOsm/kg, 270 to 500 mOsm/kg, or 270 to 400 mOsm/kg.

54-55. (canceled)


SEQUENCE LISTING (23 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 13 aa, Severe acute respiratory syndrome coronavirus 2
MFVFLVLLPLVSS

SEQ ID NO: 3 - protein, 18 aa, Artificial Sequence
  source: /note=“Description of Artificial Sequence Synthetic peptide”
MPLYKLLNVLWLVAVSNA

SEQ ID NO: 4 - protein, 1198 aa, Severe acute respiratory syndrome coronavirus 2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIK

SEQ ID NO: 5 - protein, 4 aa, Severe acute respiratory syndrome coronavirus 2
RRAR

SEQ ID NO: 6 - protein, 4 aa, Artificial Sequence
  source: /note=“Description of Artificial Sequence Synthetic peptide”
GSAS

SEQ ID NO: 7 - protein, 27 aa, Unknown
  source: /note=“Description of Unknown Foldon sequence”
GYIPEAPRDGQAYVRKDGEWVFLSTFL

SEQ ID NO: 8 - DNA, 81 bp, Unknown
  source: /note=“Description of Unknown Foldon sequence”
ggttatattcctgaagctccaagagatgggcaagcttacgttcgtaaagatggcgaatgggtattcctttctaccttttta

SEQ ID NO: 9 - DNA, 81 bp, Artificial Sequence
  source: /note=“Description of Artificial Sequence Synthetic oligonucleotide”
ggttatataccagaggctcctagagatggccaagcatacgtgcgcaaagatggtgaatgggtctttctcagcacattctta

SEQ ID NO: 10 - protein, 1243 aa, Artificial Sequence
  source: /note=“Description of Artificial Sequence Synthetic polypeptide”
MPLYKLLNVLWLVAVSNAQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGYIPEAPRDGQAYVRKDGEWVFLSTFL

SEQ ID NO: 11 - protein, 17 aa, Unknown
  source: /note=“Description of Unknown Chitinase signal sequence”
MLYKLLNVLWLVAVSNA

SEQ ID NO: 12 - DNA, 41 bp, Artificial Sequence
  source: /note=“Description of Artificial Sequence Synthetic oligonucleotide”
ctgttttcgtaacagttttgtaataaaaaaacctataaata

SEQ ID NO: 13 - protein, 1240 aa, Artificial Sequence
  source: /note=“Description of Artificial Sequence Synthetic polypeptide”
MPLYKLLNVLWLVAVSNAQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGYIPEAPRDGQAYVRKDGEWVFLSTFL

SEQ ID NO: 14 - DNA, 60 bp, Artificial Sequence
  source: /note=“Description of Artificial Sequence Synthetic oligonucleotide”
taaataatgcccttgtacaaattgttaaacgttttgtggttggtcgccgttagtaacgcg

SEQ ID NO: 15 - DNA, 60 bp, Artificial Sequence
  source: /note=“Description of Artificial Sequence Synthetic oligonucleotide”
attgttaaacgttttgtggttggtcgccgttagtaacgcgcagtgtgttaatcttacaac

SEQ ID NO: 16 - DNA, 60 bp, Artificial Sequence
  source: /note=“Description of Artificial Sequence Synthetic oligonucleotide”
ctcctactaaattaaatgatctctgctttactaatgtctatgcagattcatttgtaatta

SEQ ID NO: 17 - DNA, 60 bp, Artificial Sequence
  source: /note=“Description of Artificial Sequence Synthetic oligonucleotide”
ctctgctttactaatgtctatgcagattcatttgtaattagaggtgatgaagtcagacaa

SEQ ID NO: 18 - DNA, 60 bp, Artificial Sequence
  source: /note=“Description of Artificial Sequence Synthetic oligonucleotide”
ttcacaaatattaccagatccatcaaaaccaagcaagaggtcatttattgaagatctact

SEQ ID NO: 19 - DNA, 60 bp, Artificial Sequence
  source: /note=“Description of Artificial Sequence Synthetic oligonucleotide”
catcaaaaccaagcaagaggtcatttattgaagatctacttttcaacaaagtgacacttg

SEQ ID NO: 20 - DNA, 57 bp, Artificial Sequence
  source: /note=“Description of Artificial Sequence Synthetic oligonucleotide”
atgagcaggtatataaatgagtaattaattaagtaccgactctgctgaagaggagga

SEQ ID NO: 21 - DNA, 57 bp, Artificial Sequence
  source: /note=“Description of Artificial Sequence Synthetic oligonucleotide”
gtaattaattaagtaccgactctgctgaagaggaggaaattctccttgaagtttccc

SEQ ID NO: 22 - DNA, 56 bp, Artificial Sequence
  source: /note=“Description of Artificial Sequence Synthetic oligonucleotide”
tattcctttctacctttttataattaattaagtaccgactctgctgaagaggagga

SEQ ID NO: 23 - DNA, 56 bp, Artificial Sequence
  source: /note=“Description of Artificial Sequence Synthetic oligonucleotide”
taattaattaagtaccgactctgctgaagaggaggaaattctccttgaagtttccc